(19) United States
(12) Patent Application Publication
(10) Pub. No.: US 2026/0216513 A1
(43) Pub. Date: Jul. 30, 2026

(54) DETECTING SLEEP ONSET AND WAKE

(71) Applicant: INSPIRE MEDICAL SYSTEMS, INC., Golden Valley, MN (US)

(72) Inventors: Maxwell P. Lundeen, Minneapolis, MN (US); Meghna Singh, Eden Prairie, MN (US); Kent Lee, Minneapolis, MN (US); James Manne-Nicholas, Minneapolis, MN (US); Christopher Thorp, Minneapolis, MN (US); Mark Schatza, Golden Valley, MN (US); Ross Peter Jones, Cambridge (GB); Carlos Antonio Galeano Rios, Cambridge (GB)

(73) Assignee: INSPIRE MEDICAL SYSTEMS, INC., Golden Valley, MN (US)

(21) Appl. No.: 19/143,466

(22) PCT Filed: Dec. 20, 2023
(86) PCT No.: PCT/US2023/085152

§ 371 Date: Jun. 26, 2025

Related U.S. Application Data
(60) Provisional application No. 63/477,685, filed on Dec. 29, 2022.

(51) Int. Cl. A61N 1/36

(52) U.S. Cl. CPC A61N 1/3611; A61N 1/36139

(57) ABSTRACT

A device and/or method to detect a second instance of sleep onset within a nightly treatment period according to a second criteria different from a first criteria associated with a first instance of sleep onset during the nightly treatment period.

### BACKGROUND

A significant portion of the population suffers from various forms of sleep-related issues, some of which may involve sleep disordered breathing (SDB) and/or other conditions. In some patients, external breathing therapy devices and/or mere surgical interventions may fail to treat the sleep disordered breathing behavior.

### BRIEF DESCRIPTION OF THE DRAWINGS

FIG. 1A is a diagram schematically representing an example method of detecting sleep.

FIG. 1B is a diagram including a front view schematically representing a patient's body including example implantable components and example external elements of example methods and/or example devices.

FIG. 1C is a schematic diagram of a control portion.

FIG. 2 is a diagram schematically representing an example timeline of sleep-wake-related events according to an example method of sleep-wake determination.

FIGS. 3A and 3B are charts representing a measured signal versus threshold values for determining a sleep-wake status.

FIGS. 4A-4E are diagrams schematically representing an example method for determining a sleep-wake status.

FIGS. 5A-5B are diagrams schematically representing another example method for determining a sleep-wake status.

FIGS. 6A-6F are diagrams schematically representing yet other example methods for determining a sleep-wake status.

FIG. 7 is a diagram schematically representing yet another example method for determining a sleep-wake status.

FIG. 8 is a diagram schematically representing an example method of sensing physiologic information via sensing motion.

FIGS. 9A and 9B are diagrams schematically representing an example method of determining a sleep-wake status relative to posture information.

FIG. 9C is a diagram schematically representing an example method of determining a sleep-wake status regarding different example sensed physiologic parameters.

FIG. 10 is a flow diagram schematically representing an example method of detecting sleep and/or maintaining stimulation therapy.

FIG. 11 is a diagram schematically representing an example method of detecting sleep.

FIG. 12 is a diagram schematically representing an example method including distinguishing body motion, posture, etc.

FIGS. 13-16 are diagrams schematically representing an example method of determining a sleep-wake status relative to respiratory phase information.

FIG. 17 is a flow diagram schematically representing an example method of determining a sleep-wake status regarding variability in physiologic signals/information.

FIGS. 18-20 are diagrams schematically representing an example method of determining a sleep-wake status relative to example motion information.

FIGS. 21-22 are diagrams schematically representing an example method of determining a sleep-wake status via identifying variability in sensed physiologic information relative to a threshold.

FIGS. 23 and 24 are diagrams schematically representing an example method of determining a sleep-wake status via tracking parameters relating to time, activity, non-movement parameters, etc.

FIG. 25 is a diagram schematically representing an example method of determining a sleep-wake status according to a probability of sleep and/or a probability of wakefulness.

FIG. 26A is a diagram schematically representing an example method of determining a sleep-wake status regarding taking an action based on a probability of sleep and/or a probability of wakefulness.

FIGS. 26B-26H are diagrams schematically representing an example taking an action, in relation to a method of determining a sleep-wake status, including initiating or terminating stimulation, relative to various example boundaries regarding time, temperature, sleep stages, etc.

FIG. 26I is a diagram schematically representing an example method of receiving inputs regarding some of the example boundaries represented in at least FIGS. 26B-26H.

FIG. 27 is a diagram schematically representing an example method of determining a sleep-wake status including dividing sense signal(s) to enable assessing different sleep-wake determination parameters.

FIG. 28 is a diagram schematically representing an example method of determining a sleep-wake status according to a probability of sleep and/or a probability of wakefulness.

FIGS. 29A and 29B are diagrams schematically representing an example method of determining a sleep-wake status according to wakefulness information and snoring information, respectively.

FIG. 30A is a block diagram schematically representing an example sensing portion of an example device and/or used as part of example method for determining a sleep-wake status.

FIG. 30B is a block diagram schematically representing an example processing portion, which may form part of and/or be in communication with the example sensing portion.

FIG. 31A is a diagram including a front view schematically representing a patient's body and example implanted medical device for treating sleep disordered breathing and/or determining a sleep-wake status.

FIG. 31AA is a diagram including a front view schematically representing an example implantable medical device with sensors.

FIGS. 31B-31F are diagrams schematically representing different example implementations of example implantable medical devices as a microstimulator implanted in a head-and-neck region.

FIG. 32 is a block diagram schematically representing an example care engine.

FIG. 33 is a diagram schematically representing an example respiratory pattern.

FIGS. 34A and 34B are block diagrams schematically representing example control portions.

FIG. 35 is a block diagram schematically representing an example user interface.

FIG. 36 is a block diagram schematically representing example communication arrangements between an implantable medical device and external devices.

FIG. 37A is a diagram schematically representing an example user interface including example therapy usage patterns, sleep-wake status, sleep quality portions, use metrics, etc., which may be used in association with example method and/or example device for determining sleep-wake status.

FIGS. 37B and 37C are diagrams schematically representing example methods including taking an action in relation to a sleep-wake status determination.

FIG. 38 is a diagram schematically representing an example method of receiving input in relation to starting and/or stopping therapeutic treatment.

FIG. 39 is a diagram schematically representing an example method including tracking information regarding usage, starting, stopping, etc. of a therapy.

FIG. 40 is a diagram schematically representing an example method and/or example device including a medical device in relation to a resource for determining a sleep-wake status and/or sleep latency onset information (e.g. variability, etc.), including training a data model, etc.

FIG. 41 is a diagram schematically representing an example method and/or example device for training a data model regarding determining a sleep-wake status.

FIG. 42 is a diagram schematically representing an example method and/or example device for determining a sleep-wake status according to a trained data model.

FIG. 43 is a chart schematically representing an example motion signal of a patient over 90 minutes.

FIG. 44A is a chart schematically representing an example motion signal for detecting sleep onset of a patient.

FIG. 44B is a chart schematically representing an example motion signal for detecting wake after sleep onset (WASO) of a patient.

FIGS. 45A and 45B are diagrams schematically representing example methods for determining sleep onset of a patient.

### DETAILED DESCRIPTION

In the following detailed description, reference is made to the accompanying drawings which form a part hereof, and in which is shown by way of illustration specific examples in which the disclosure may be practiced. It is to be understood that other examples may be utilized and structural or logical changes may be made without departing from the scope of the present disclosure. The following detailed description, therefore, is not to be taken in a limiting sense. It is to be understood that features of the various examples described herein may be combined, in part or whole, with each other, unless specifically noted otherwise.

At least some examples of the present disclosure are directed to devices for diagnosis, therapy, and/or other care of medical conditions. At least some examples may comprise implantable devices and/or methods comprising use of implantable devices. However, in some examples, the methods and/or devices may comprise at least some external components. In some examples, a therapeutic medical device may comprise a combination of implantable components and external components.

At least some of the example devices and/or example methods may relate to detecting sleep onset and wake, the results of which may be used in caring for a patient such as (but not limited to) diagnosing, evaluating, monitoring, and/or treating a wide variety of patient conditions.

At least some of the example devices and/or example methods may relate to sleep disordered breathing (SDB) care, which may comprise monitoring, diagnosis, evaluation, and/or treatment, which may comprise stimulation in some examples.

In some examples, providing patient care may comprise automatically determining a sleep-wake status, which may in turn comprise detecting sleep and/or detecting wakefulness. In some examples, detecting sleep comprises detecting an onset of sleep. In some such examples, the sleep-wake determination may be used to initiate (and/or maintain) a treatment period such as (but not limited to) treatments in which neurostimulation therapy is used to treat sleep disordered breathing. In some of these examples, sleep-wake determination may comprise determining an initial sleep onset, wake after sleep onset (WASO), and/or sleep onset after WASO to automatically activate stimulation during sleep and automatically deactivate the stimulation during wake. In some examples, the same inputs, sleep-related signals, parameters, etc. may be used to detect each of initial sleep onset, wake after sleep onset (WASO), and/or sleep onset after WASO. In some examples, different inputs, sleep-related signals, parameters, etc. may be used to detect each of initial sleep onset, wake after sleep onset (WASO), and/or sleep onset after WASO. In addition, the threshold values, criteria, etc. associated with each input, sleep-related signal, parameter, etc. used to detect initial sleep onset, wake after sleep onset (WASO), and/or sleep onset after WASO may be different.

Different patients have different sleep habits, including when they fall asleep and when they wake up during the night. This phenomenon is present, whether a patient has sleep disordered breathing and/or conditions. A patient falling asleep at the start of the night behaves differently than a patient waking up during the night. This leads to a difference in signals that leads to difficulty in setting a single threshold or criteria for detecting a sleep-wake status during the entire night. Therefore, as disclosed herein, multiple (e.g. at least two) thresholds or criteria may be used for detecting a sleep-wake status during the night.

Patients may get anxiety around stimulation activating before they fall asleep, or waking up in the middle of the night and feeling the stimulation. By automatically activating/deactivating stimulation based on the sleep-wake status of the patient, use of the stimulation device may increase since the patient does not need to manually activate the stimulation device, which may lead to better outcomes for the patient. In addition, by automatically activating/deactivating stimulation based on the sleep-wake status of the patient, use of the stimulation device may increase because the patient will no longer worry about actually falling asleep within a predetermined window (e.g. 30 minutes) before stimulation would begin. By detecting when the patient is asleep and awake, automatic activation/deactivation of stimulation may be implemented. The inputs, sleep-related signals, parameters, etc. and associated threshold values, criteria, etc. used to detect initial sleep onset, wake after sleep onset (WASO), and/or sleep onset after WASO may be different from one patient to another. Thus, the inputs, sleep-related signals, parameters, etc. and associated threshold values, criteria, etc. used to detect the sleep-wake status of a particular patient may be individualized for the patient.

At least some examples of determining a sleep-wake status also may relate to cardiac care, drug delivery, pelvic-related care, and/or other forms of care, whether standing alone or in association with sleep disordered breathing (SDB) care.

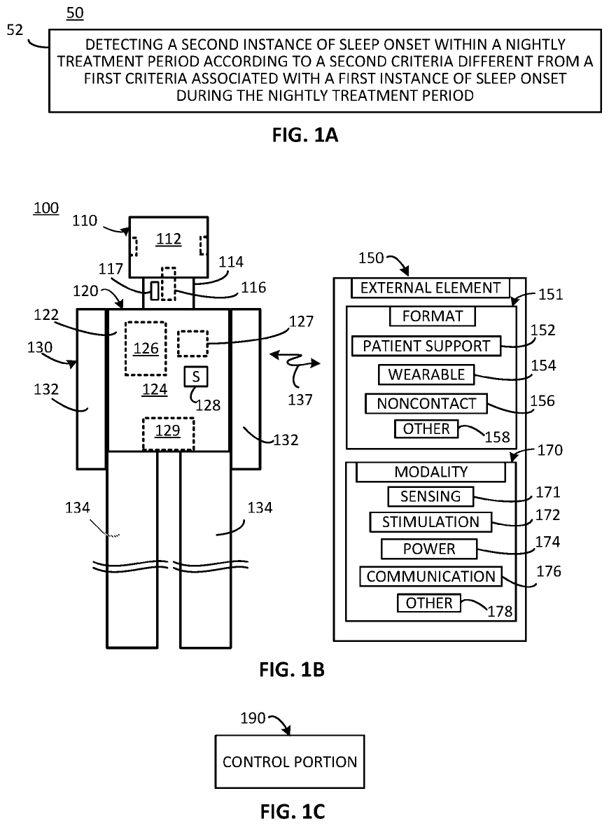

These examples, and additional examples, are further described in association with at least FIGS. 1A-42.

As schematically represented at 50 in FIG. 1A, in some examples a method 52 comprises detecting a second instance of sleep onset within a nightly treatment period according to a second criteria different from a first criteria associated with a first instance of sleep onset during the nightly treatment period. In some examples, the first instance of sleep onset may be an initial sleep onset at the beginning of the nightly treatment period, and the second instance of sleep onset may be a sleep onset after WASO. The first criteria may include a first input, sleep-related signal, or parameter and/or a corresponding first threshold value. The second criteria may include a second input, sleep-related signal, or parameter and/or a corresponding second threshold value different from the first input, sleep-related signal, or parameter and/or the first threshold value.

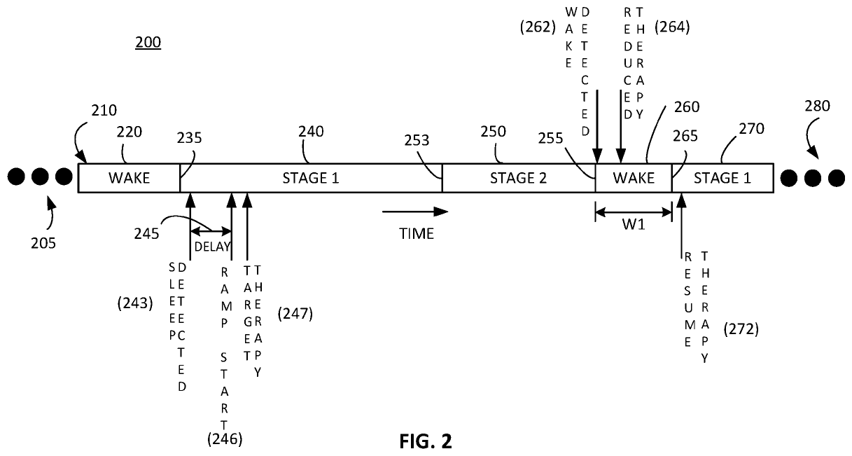

As further described below in association with at least FIGS. 2-42, in some examples determining a sleep-wake status may be associated with and/or form part of a method of determining an initial sleep onset based on first sleep-wake determination parameters and corresponding first threshold values. In some examples, the example method may further comprise determining a wake after sleep onset (WASO) based on second sleep-wake determination parameters and corresponding second threshold values. In some examples, the example method may further comprise determining sleep onset after WASO based on third sleep-wake determination parameters and corresponding third threshold values. The first, second, and third sleep-wake determination parameters for determining each of initial sleep onset, wake after sleep onset (WASO), and sleep onset after WASO may be the same or different. The third threshold values may be different from the first and/or second threshold values.

FIG. 1B is a block diagram schematically representing a patient's body 100, including example target portions 110-134 at which at least some example sensing element(s) and/or stimulation elements may be employed to implement at least some examples of the present disclosure.

As shown in FIG. 1B, patient's body 100 comprises a head-and-neck portion 110, including head 112 and neck 114. Head 112 comprises cranial tissue, nerves, etc., and upper airway 116 (e.g. nerves, muscles, tissues), etc. As further shown in FIG. 1B, the patient's body 100 comprises a torso 120, which comprises various organs, muscles, nerves, other tissues, such as but not limited to those in pectoral region 122 (e.g. lungs 126, cardiac 127), abdomen 124, and/or pelvic region 129 (e.g. urinary/bladder, anal, reproductive, etc.). As further shown in FIG. 1B, the patient's body 100 comprises limbs 130, such as arms 132 and legs 134.

It will be understood that various sensing elements (and/or stimulation elements) as described throughout the various examples of the present disclosure may be deployed within the various regions of the patient's body 100 to sense and/or otherwise diagnose, monitor, treat various physiologic conditions such as, but not limited to those examples described below in association with FIGS. 2-42. In some such examples, a stimulation element 117 may be located in or near the upper airway 116 for treating sleep disordered breathing (and/or near other nerves/muscles for treating other conditions) and/or a sensing element 128 may be located anywhere within the neck 114 and/or torso 120 (or other body regions) to sense physiologic information for providing patient care (e.g. SDB, other) with the sensed physiologic information including, but not limited to, sleep onset detection and related parameters.

In some examples, at least a portion of the stimulation element 117 may comprise part of an implantable component/device, such as an implantable pulse generator (IPG) whether full sized or sized as a microstimulator. The implantable components (e.g. IPG, other) may comprise a stimulation/control circuit, a power supply (e.g. non-rechargeable, rechargeable), communication elements, and/or other components. In some examples, the stimulation element 117 also may comprise a stimulation electrode and/or stimulation lead connected to the implantable pulse generator.

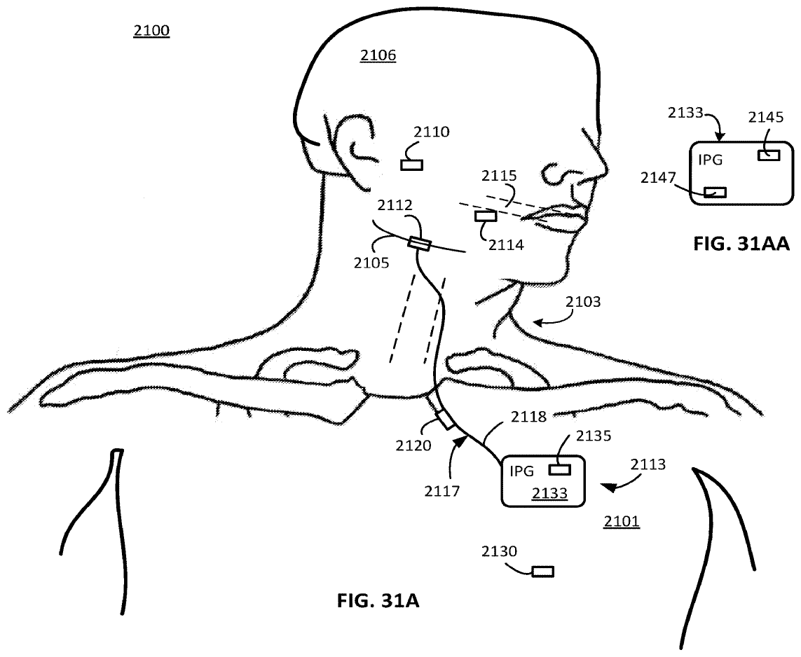

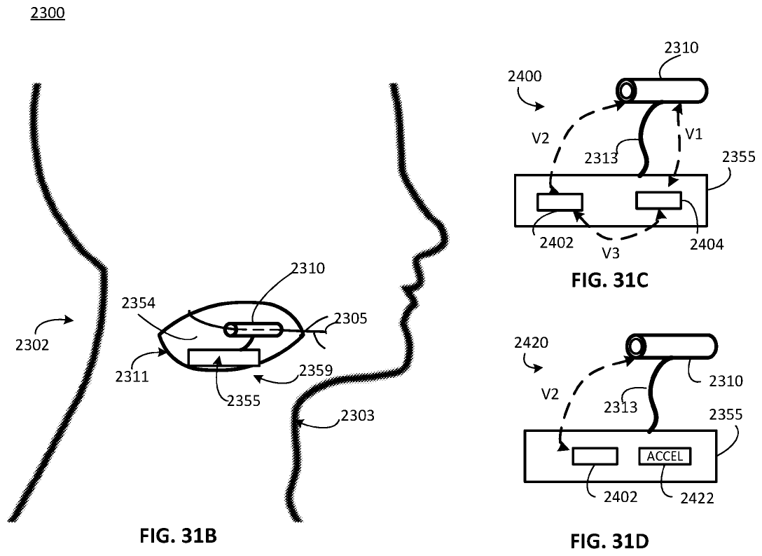

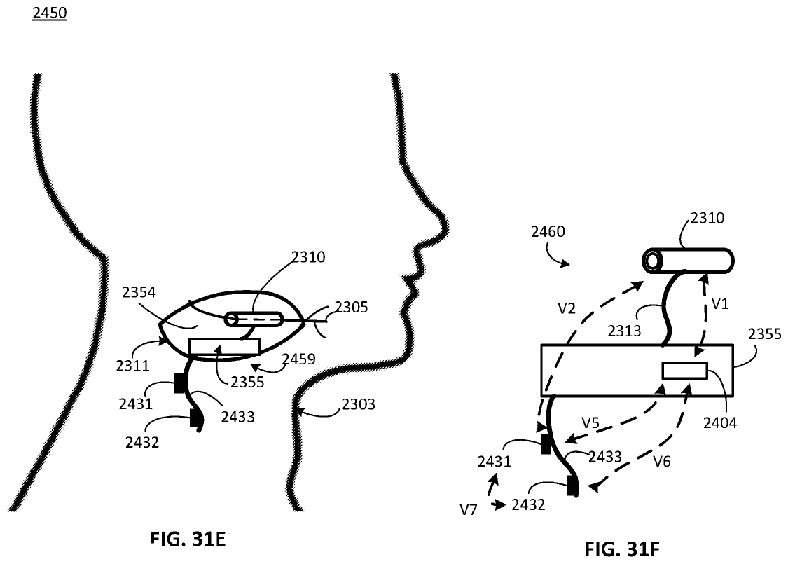

Further details regarding a location, structure, operation, and/or use of the sensing element 128, external element(s) 150, and/or stimulation element 117 are described below in association with at least FIGS. 1C-42, and in particular, at least FIGS. 31A-31F.

In some examples, at least a portion of the stimulation element 117 may comprise part of an external component/device such as, but not limited to, the external component comprising a pulse generator (e.g. stimulation/control circuitry), power supply (e.g. rechargeable, non-rechargeable), and/other components. In some examples, a portion of the stimulation element 117 may be implantable and a portion of the stimulation element 117 may be external to the patient.

Accordingly, as further shown in FIG. 1B, the various sensing element(s) 128 and/or stimulation element(s) 117 implanted in the patient's body may be in wireless communication (e.g. connection 137) with at least one external element 150.

As further shown in FIG. 1B, in some examples, the external element(s) 150 may be implemented via a wide variety of formats such as, but not limited to, at least one of the formats 151 including a patient support 152 (e.g. bed, chair, sleep mat, other), wearable elements 154 (e.g. finger, wrist, head, neck, shirt), noncontact elements 156 (e.g. watch, camera, mobile device, other), and/or other elements 158.

As further shown in FIG. 1B, in some examples, the external element(s) 150 may comprise one or more different modalities 170 such as (but not limited to) a sensing portion 171, stimulation portion 172, power portion 174, communication portion 176, and/or other portion 178. The different portions 171, 172, 174, 176, 178 may be combined into a single physical structure (e.g. package, arrangement, assembly), may be implemented in multiple different physical structures, and/or with just some of the different portions 171, 172, 174, 176, 178 combined together in a single physical structure.

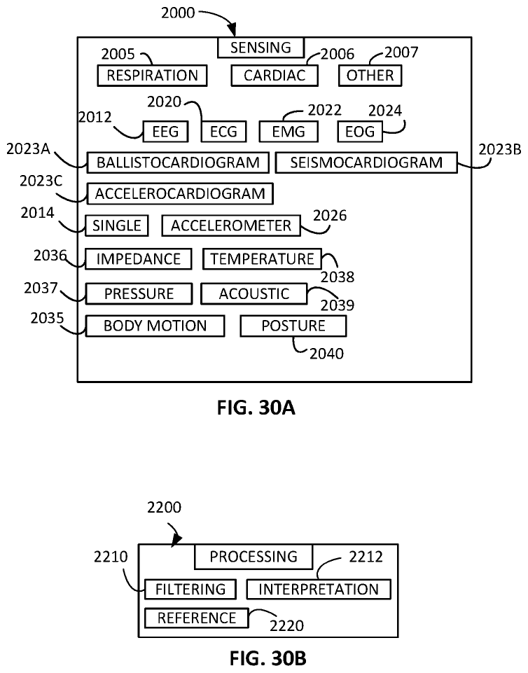

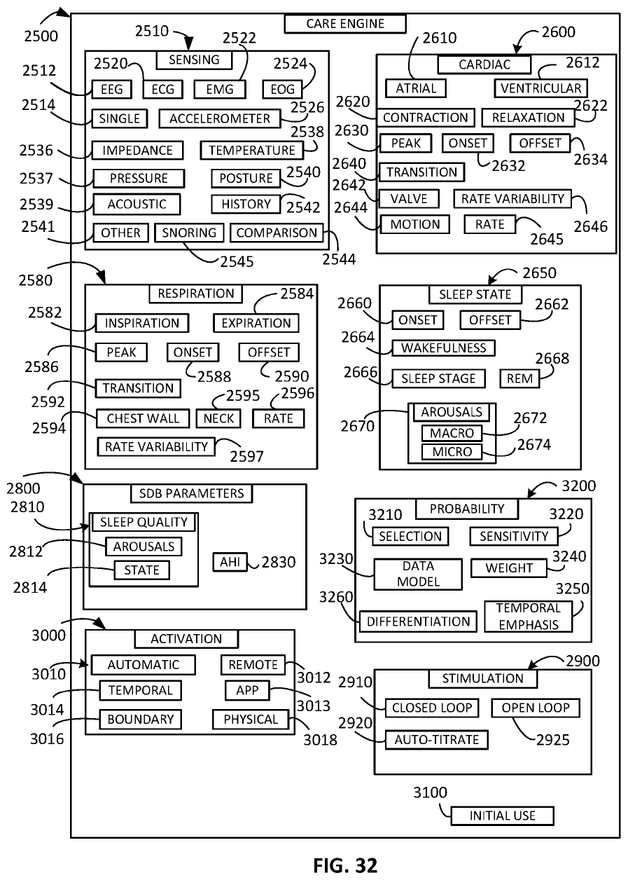

Among other such details, in some examples the external sensing portion 171 and/or implanted sensing element 128 may comprise at least some of substantially the same features and attributes of at least sensing portion 2000 and/or care engine 2500, as further described below in FIGS. 30A and 32, respectively.

In some examples, the external stimulation portion 172 and/or implanted stimulation element 117 may comprise at least some of substantially the same features and attributes of at least the stimulation arrangements, as further described below in association with at least FIGS. 31A-31F, 32 and/or other examples throughout the present disclosure.

In some examples, the external power portion 174 and/or power components associated with implanted stimulation element 117 may comprise at least some of substantially the same features and attributes of at least the stimulation arrangements, as further described below in association with at least FIGS. 31A-11 31F, 32 and/or other examples throughout the present disclosure. In some such examples, the respective power portion, components, etc. may comprise a rechargeable power element (e.g. supply, battery, circuitry elements) and/or non-rechargeable power elements (e.g. battery). In some examples, the external power portion 174 may comprise a power source by which a power component of the implanted stimulation element 117 may be recharged.

In some examples, the wireless communication portion 176 (e.g. connection/link at 137) may be implemented via various forms of radiofrequency communication and/or other forms of wireless communication, such as (but not limited to) magnetic induction telemetry, Bluetooth (BT), Bluetooth Low Energy (BLE), near infrared (NIF), near-field protocols, Wi-Fi, Ultra-Wideband (UWB), and/or other short range or long range wireless communication protocols suitable for use in communicating between implanted components and external components in a medical device environment.

Examples are not so limited as expressed by other portion 178 via which other aspects of implementing medical care may be embodied in external element(s) 150 to relate to the various implanted and/or external components described above.

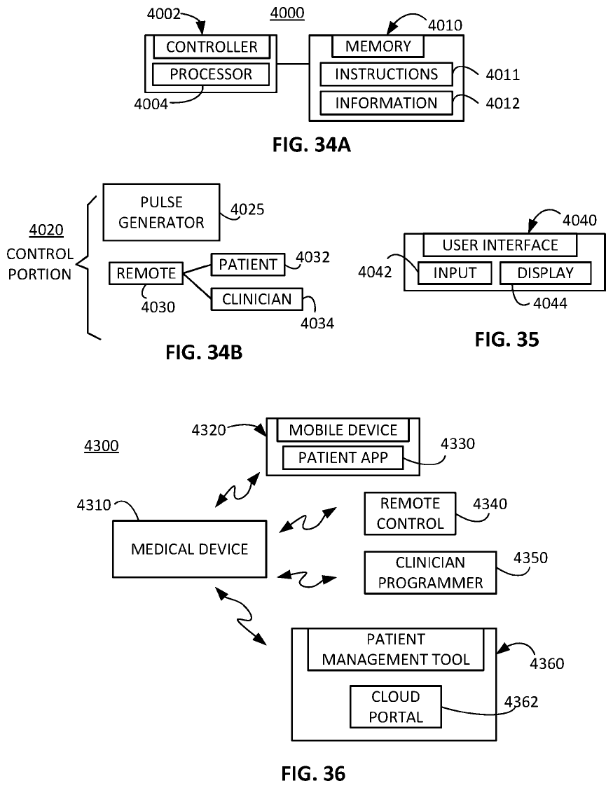

FIG. 1C schematically represents a control portion 190, which may comprise at least some of substantially the same features and attributes as the control portion 4000 in FIG. 34A and/or care engine 2500 in FIG. 32. In some examples, the control portion 190 will be part of a care engine (e.g. 2500) or the like. Among other aspects, example methods and/or example devices may be implemented via the control portion 190. In some examples, the control portion 190 may be used to implement at least some of the various example devices and/or example methods of the present disclosure as described herein. In some examples, the control portion 190 may form part of, and/or be in communication with, the sensing element 128 and/or the stimulation element 117 in FIG. 1B, external element(s) 150, and/or other medical device (or portions thereof), as further described later.

In various examples, the control portion 190 is programmed to detect a second instance of sleep onset within a nightly treatment period according to a second criteria different from a first criteria associated with a first instance of sleep onset during the nightly treatment period as represented at 50 in FIG. 1A. In various other examples, the control portion 190 is programmed to detect an initial sleep onset, wake after sleep onset (WASO), and sleep onset after WASO using the same or different inputs, sleep-related signals, parameters, etc. and associated threshold values as further described below with reference to the following figures.

FIG. 2 is a diagram schematically representing a timeline 210 of sleep-wake-related events according to an example method 200 of sleep-wake determination, such as may occur during sleep disordered breathing (SDB) care (e.g. monitoring, diagnosis, treatment, etc.). In some examples, the example SDB care may comprise at least some of substantially the same features and attributes as the example SDB care methods and/or devices (including sleep-wake detection) as described in association with FIGS. 1A-42. As shown in FIG. 2, the timeline 210 includes a series of wake and sleep periods with wake period 220 occurring just before a first sleep stage period 240 (e.g. stage 1). The wake period 220 in FIG. 2 may represent an end portion of a wake period extending since the end of a prior night's sleep or may represent another wake period.

As further represented by indicator 235, a real physiologic transition occurs between the wake period 220 and the first sleep stage 240 and indicator 243 represents a detection of sleep (also referred to as an initial sleep onset) according to examples of the present disclosure. As shown in FIG. 2, the detection of sleep (243) may occur just after the physiologic transition 235.

In some examples, the detection of sleep 243 may trigger a delay period 245 prior to a start of therapy (e.g. electrical stimulation). In some such examples, the duration of the delay generally corresponds to an amount of time sufficient for a patient to experience sufficiently sound sleep such that the patient will not be awakened by the onset of stimulation. Moreover, in some examples, once stimulation begins it may be implemented in a ramped manner (246) with an initial lower stimulation intensity which is gradually increased until a target stimulation intensity (247) is achieved to therapeutically provide electrical stimulation to an upper airway patency related tissue.

As previously noted in the present disclosure, at least some example implementations of method 200 in FIG. 2 may comprise identifying, maintaining, and/or optimizing a target stimulation intensity (e.g. therapy level) without intentionally identifying a stimulation discomfort threshold at the time of implantation or at a later point in time after implantation.

As further shown in FIG. 2, once the target stimulation intensity is achieved, it may be maintained throughout the treatment period.

In some examples, the target stimulation intensity may be automatically adjusted (e.g. auto-titrated) during the treatment period. In some such examples, the automatic adjustment of the target stimulation intensity may be implemented according to at least some of substantially the same features and/or attributes as described in association with at least auto-titration parameter 2920 in FIG. 32.

As further shown in FIG. 2, after some period of time (which may vary from night to night) the patient may sometimes experience a wake period 260 during a treatment period, which interrupts a sleep stage (e.g. a second sleep stage (S2) 250 in this example). The example method 200 detects wakefulness (262) (also referred to as wake after sleep onset (WASO)), which may extend for a period of time (W1), before the patient goes back to sleep, such as represented by sleep stage 270 and transition 265 between the respective wake period 260 and sleep stage 270 (also referred to as sleep onset after WASO).

In some examples, method 200 may completely pause stimulation during the wakeful period 260 or instead in some examples, method 200 may implement a reduced therapy 264 during the wakeful period 260 because of the expectation of the patient going back to sleep and a full stimulation therapy being resumed. In some such examples, the reduced therapy at 264 may comprise providing stimulation at a functional threshold (FT), which corresponds to a minimum amplitude at which the stimulation will cause the tongue to protrude at least part way past the lower teeth and at which a therapeutic outcome (e.g. reduction in apneas) may be achieved. However, in some such examples, the reduced therapy at 264 may comprise providing stimulation at a sensation threshold (ST), which involves a stimulation intensity less than the stimulation intensity to reach the functional threshold (FT). The sensation threshold (ST) may correspond to a minimum amplitude at which the patient can sense stimulation.

As indicated at 272 in FIG. 2, therapy may be resumed automatically. It will be understood that, in at least some examples, the resumption of therapy 272 may comprise substantially the same features and attributes as the initiation of therapy as previously described in relation to indicators 243, 246, 247 in FIG. 2, including detection of sleep 243 according to at least some of the examples of the present disclosure to determine a sleep-wake status.

In some examples, in general terms, the beginning of the first sleep stage 240 generally corresponds to a beginning of a treatment period during which a patient may be treated for sleep disordered breathing and/or the method (and/or device) may monitor for or diagnose sleep disordered breathing.

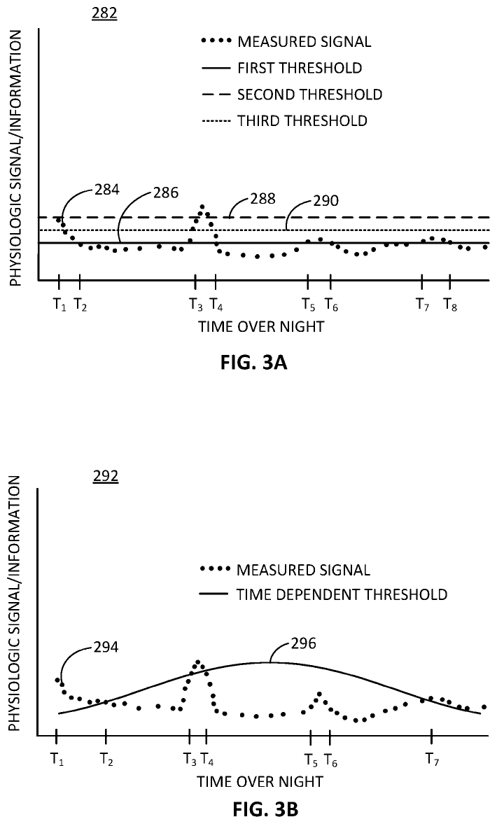

More specific example methods, devices, and/or arrangements of determining a sleep-wake status (including determining initial sleep onset, wake after sleep onset, and sleep onset after WASO and related information) are described and illustrated in association with at least FIGS. 3A-42.

FIG. 3A is a chart 282 representing a measured physiologic signal/information versus threshold values for determining a sleep-wake status. As further described below, in some examples a physiologic signal may be obtained from an accelerometer signal and/or other sensing modalities. However, in some examples, an accelerometer signal may be suited to certain types of some physiologic signals/information particularly related to sleep-wake determination such as (but not limited to) activity/motion, body position (e.g. posture), and the like. This type of information may be obtained via the accelerometer signal in addition to the accelerometer signal being used to sense other physiologic signals/information such as (but not limited to) at least respiratory and/or cardiac signals/information, which is also suited for sleep-wake determination, among other purposes. Accordingly, in some examples, the measured physiologic signals may comprise at least some physiologic signals/information obtained from an accelerometer signal and/or at least some physiologic signals/information obtained from other sensing modalities, as further described later.

A measured signal as indicated at 284, which is indicative of the sleep-wake status of a patient (e.g. 100 of FIG. 1B) may be obtained (e.g. via sensing element 128 of FIG. 1B and/or external sensor 171, 150 of FIG. 1B) throughout the night and/or a sleep period (e.g. treatment period). The measured signal 284 may be compared to a first threshold value as indicated at 286 to detect initial sleep onset. With the measured signal 284 above the first threshold value 286 at time T₁, the patient is determined to be awake. When the measured signal 284 falls below the first threshold value 286 at time T₂, the patient is determined to be asleep indicating initial sleep onset for the night and/or sleep period. In response to detecting initial sleep onset, in some examples stimulation may be activated as previously described with reference to FIG. 2.

After initial sleep onset is detected at time T₂, the measured signal 284 may be compared to a second threshold value as indicated at 288 to detect wake after sleep onset (WASO). The second threshold value 288 may be higher than the first threshold value 286. With the measured signal 284 below the second threshold value 288 between times T₂ and T₃, the patient is determined to be asleep, even if the measured signal 284 rises above the first threshold value 286 as shown just prior to time T₃. When the measured signal 284 rises above the second threshold value 288 at time T₃ (e.g. due to a macro arousal described below), the patient is determined to be awake indicating a wake after sleep onset for the night and/or sleep period. In response to detecting wake after sleep onset, in some examples stimulation may be paused as previously described with reference to FIG. 2.

It will be understood that once the patient awakes, they may stay awake for some time period (e.g. 1, 2, 5, 10, or 15 minutes) before attempting to go back to sleep. For illustrative simplicity, the measured signal 284 in FIG. 3A schematically represents the wake period (above threshold 228) as a single or few data points even though in actuality the wake period may last at least a few minutes or longer, in some examples.

After a wake after sleep onset (WASO) is detected at time T₃, the measured signal 284 may be compared to a third threshold value as indicated at 290 to detect sleep onset after WASO. In some examples, the third threshold value 290 may be lower than the second threshold value 288. In other examples, the third threshold value 290 may equal the second threshold value 288. The third threshold value 290 may be higher than the first threshold value 286 since falling asleep in the middle of the night or sleep period after waking is typically easier/faster than initially falling asleep at the beginning of the night or sleep period. With the measured signal 284 above the third threshold value 290 between times T₃ and T₄, the patient is determined to be awake. When the measured signal 284 falls below the third threshold value 290 at time T₄, the patient is determined to be asleep indicating sleep onset after WASO for the night and/or sleep period. In response to detecting sleep onset after WASO, in some examples stimulation may be resumed as previously described with reference to FIG. 2.

As indicated between times T₅ and T₆ and between times T₇ and T₈, the measured signal 284 rises above the first threshold value 286 (e.g. due to micro arousals described below), however, the patient is still determined to be asleep since the measured signal 284 does not rise above the second threshold value 288 indicating a wake after sleep onset. At the end of the night or sleep period, sometime after time T₈, the measured signal 284 may rise above the second threshold value 288 indicating the patient is awake. In response to detecting the wake state at the end of the night or sleep period, in some examples stimulation may be deactivated until the next night or sleep period.

As further described below, in some examples the first threshold value 286, the second threshold value 288, and the third threshold value 290 may be initially selected based on a general population model. In some examples, the first threshold value 286, the second threshold value 288, and the third threshold value 290 may then be adjusted based on patient feedback to individualize the threshold values to the patient.

While FIG. 3A illustrates a single measured signal and threshold values for the single measured signal to detect initial sleep onset, wake after sleep onset (WASO), and sleep onset after WASO, in other examples multiple measured signals and corresponding threshold values for each of the multiple measured signals may be used in combination to detect initial sleep onset, wake after sleep onset, and sleep onset after WASO as further described below. In addition, while a higher measured signal 284 in FIG. 3A indicates wakefulness and a lower measured signal 284 in FIG. 3A indicates sleepfulness, in other examples a higher measured signal may indicate sleepfulness and a lower measured signal may indicate wakefulness. In this case, the first threshold value, the second threshold value, and the third threshold value may be inverted. That is, the first threshold value may be greater than the second threshold value and the third threshold value, and the second threshold value may be less than the third threshold value.

FIG. 3B is a chart 292 representing a measured physiologic signal/information versus a time dependent threshold value for determining a sleep-wake status. In some examples, a time dependent threshold value may be used since the probability of falling asleep increases as a patient has attempted sleep for some amount of time, or increases if the patient has already slept for some given number of hours. The longer a patient attempts to fall asleep, the more likely the patient will eventually fall asleep. Further, given that a patient has slept for some given number of hours, if wake after sleep onset occurs, then it is easier for the patient to fall back asleep. Thus, the time dependent threshold value may account for these probabilities.

A measured signal as indicated at 294, which is indicative of the sleep-wake status of a patient (e.g. 100 of FIG. 1B) may be obtained (e.g. via sensing element 128 of FIG. 1B and/or external sensor 171, 150 of FIG. 1B) throughout the night and/or a sleep period. The measured signal 294 may be compared to a time dependent threshold value as indicated at 296 to detect initial sleep onset, wake after sleep onset, and/or sleep onset after WASO. Time dependent threshold 296 is initially at a minimum value at the start of the night and/or sleep period, increases from the minimum value to a maximum value, and then decreases from the maximum value back to the minimum value by the end of the night and/or sleep period.

With the measured signal 294 above the time dependent threshold value 296 at time T₁, the patient is determined to be awake. When the measured signal 294 falls below the time dependent threshold value at time T₂, the patient is determined to be asleep indicating initial sleep onset for the night or sleep period. With the measured signal 294 below the time dependent threshold value 296 between times T₂ and T₃, the patient is determined to be asleep. When the measured signal 294 rises above the time dependent threshold value 296 at time T₃ (e.g. due to a macro arousal described below), the patient is determined to be awake indicating a wake after sleep onset for the night or sleep period. With the measured signal 294 above the time dependent threshold value 296 between times T₃ and T₄, the patient is determined to be awake. As previously noted in association with FIG. 3A, it will be understood that once the patient awakes, they may stay awake for some time period (e.g. 1, 2, 5, 10, or 15 minutes) before attempting to go back to sleep. For illustrative simplicity, the measured signal 294 in FIG. 3B schematically represents the wake period (above threshold 296) as a single or few data points even though in reality the wake period may last at least a few minutes or longer, in some examples.

When the measured signal 294 falls below the time dependent threshold value 296 at time T₄, the patient is determined to be asleep indicating sleep onset after WASO for the night or sleep period. With the measured signal 294 below the time dependent threshold value 296 between times T₄ and T₇, the patient is determined to be asleep. It is noted that at least between times T₅ and T₆, based on the threshold value 296 at time T₁, the patient would have been determined to be awake. However, since the threshold value 296 is higher between times T₅ and T₆ than at time T₁, the patient is determined to be asleep between times T₅ and T₆. Once the measured signal 294 rises above the time dependent threshold value 296 at time T₇, the patient is determined to be awake, which may be at or near the end of the night or sleep period.

In some examples, time dependent threshold value 296 may be used for first threshold value 286, second threshold value 288, and/or third threshold value 290 of FIG. 3A. While FIG. 3B illustrates a single measured signal and time dependent threshold value for the single measured signal to detect initial sleep onset, wake after sleep onset, and sleep onset after WASO, in other examples multiple measured signals and corresponding time dependent threshold values for each of the multiple measured signals may be used in combination to detect initial sleep onset, wake after sleep onset, and sleep onset after WASO as further described below. In addition, while a higher measured signal 294 in FIG. 3B indicates wakefulness and a lower measured signal 294 in FIG. 3B indicates sleepfulness, in other examples a higher measured signal may indicate sleepfulness and a lower measured signal may indicate wakefulness. In this case, the time dependent threshold value 296 may be inverted, such that the time dependent threshold value is initially at a maximum value at the start of the night or sleep period, decreases from the maximum value to a minimum value, and then increases from the minimum value back to the maximum value by the end of the night and/or sleep period.

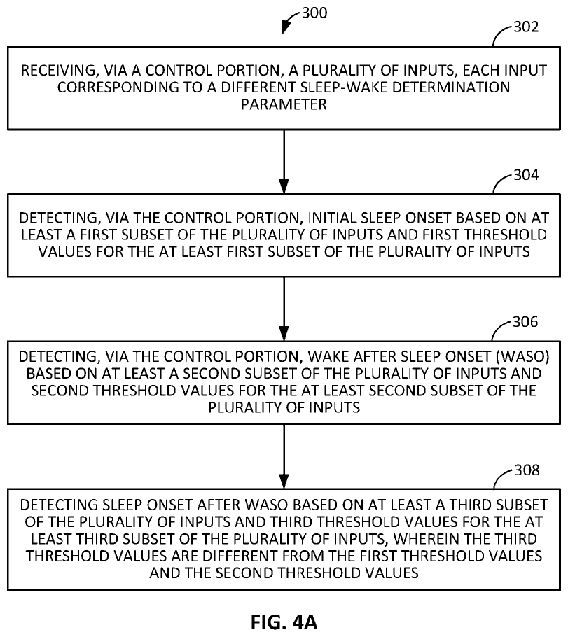

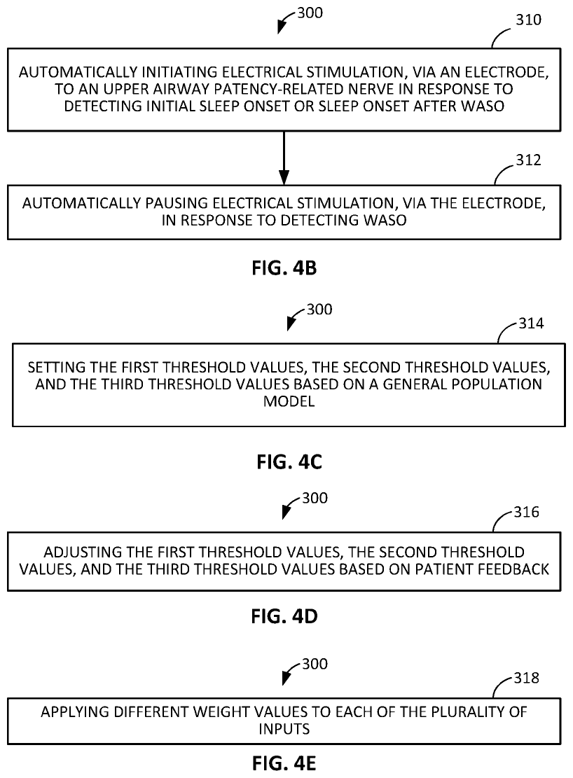

FIGS. 4A-4E are diagrams schematically representing an example method 300 for determining a sleep-wake status. Method 300 may be implemented by a stimulation element 117, sensing element 128, external element 150, and/or control portion 190 of FIGS. 1B-1C. As illustrated in FIG. 4A at 302, method 300 may include receiving, via a control portion (e.g. 190), a plurality of inputs, each input corresponding to a different sleep-wake determination parameter. At 304, method 300 may include detecting, via the control portion, initial sleep onset based on at least a first subset of the plurality of inputs and first threshold values for the at least first subset of the plurality of inputs. At 306, method 300 may include detecting, via the control portion, wake after sleep onset (WASO) based on at least a second subset of the plurality of inputs and second threshold values for the at least second subset of the plurality of inputs. At 308, method 300 may include detecting sleep onset after WASO based on at least a third subset of the plurality of inputs and third threshold values for the at least third subset of the plurality of inputs, wherein the third threshold values are different from the first threshold values and the second threshold values.

In some examples, the first threshold values and/or the third threshold values are time dependent (e.g. as indicated by 296 in FIG. 3B). For example, during a sleep period, the third threshold values may be initially at minimum values at a start of the sleep period, increase from the minimum values to maximum values, and then decrease from the maximum values back to the minimum values by the end of the sleep period. In some examples, the second subset of the plurality of inputs is the same as the first subset of the plurality of inputs. In other examples, the second subset of the plurality of inputs is different from the first subset of the plurality of inputs. For example, the first subset of the plurality of inputs may comprise at least one of heart rate variability or body temperature, and the second subset of the plurality of inputs may comprise at least one of activity or locomotor inactivity during sleep (LIDS).

In some examples, the first subset of the plurality of inputs is the same as the third subset of the plurality of inputs. In other examples, the first subset of the plurality of inputs may be different from the third subset of the plurality of inputs. In some examples, the third subset of the plurality of inputs may comprise at least one of activity, locomotor inactivity during sleep (LIDS), a light sensor signal, or time elapsed since initial sleep onset.

In one example, the plurality of inputs may comprise an accelerometer sensor signal, such as an angle of the accelerometer. In some examples, the plurality of inputs may comprise at least one of the following: a physiologic signal/information obtained from an accelerometer sensor signal or sensing modalities, an environmental signal, time, or patient information. In some examples, the plurality of inputs may comprise at least one of the following physiologic signals/information:
respiratory signal/information (e.g. respiration rate, respiration rate variability), electromyography (EMG), microneurography, cardiac signal/information (e.g. heart rate, heart rate variability), body temperature, posture, activity, or locomotor inactivity during sleep (LIDS). In yet further examples, the plurality of inputs may comprise at least one of the following: geolocation, proximity to sleeping area (e.g. in bedroom, in bed, etc.), light sensor signal, noise sensor signal, movement sensor signal (e.g. a physiologic signal/information), patient electronic device proximity and/or charge status (e.g. proximity and/or charging may indicate nighttime or sleep period), time elapsed since initial sleep onset (e.g. involving physiologic signal/information, in some examples, or patient circadian rhythm (e.g. physiologic signal/information). In yet further examples, the plurality of inputs may comprise at least one of the following patient inputs: meal intake, meal timing, indicated sleepiness, demographics, or comorbidities. In yet further examples, the plurality of inputs may comprise any suitable combination of the inputs described above.

In some such examples, at least some of the inputs (e.g. physiologic signals, related physiologic information, other) may be obtained via an accelerometer signal such as, but not limited to, respiratory information (e.g. rate, rate variability), cardiac information (e.g. heart rate, heart rate variability), activity, motion, body position (e.g. posture), LIDS, movement, and/or other physiologic signals and related physiologic information. The accelerometer signal may be obtained from an implanted accelerometer (e.g. forming part of stimulation element 117 or sensing element 128 of FIG. 1B) and/or from an external accelerometer (e.g. 171, 150 of FIG. 1B).

Inputs relating to activity, motion, body position and movement can relate to patterns of accelerometer signals over a period of time. LIDS is an example of this, where an activity count in a window of time is determined, the quantity 100/(1+activity count) is calculated and then smoothed with an averaging filter. A high value of LIDS corresponds to a low level of activity sustained over a period of time, which is indicative of sleep onset. In another example, the activity count could be weighted by the magnitude of each motion. In this way, a few, large motions provide just as much indication of wakefulness as a larger number of small motions. In another example, the activity count can include small motion over a short window of time and large motions over a long window of time. In this way, a threshold value can only be exceeded a long time after a large motion or a shorter time after a smaller motion. This can reflect the way that a patient's motion diminishes in magnitude as they approach sleep onset.

Similarly, an input can relate to a pattern of activity over a period of time that is associated with wakefulness. A single, large motion could correspond to either a minor arousal or waking up but repeated, large motions are more generally associated with waking. So, an input can include an activity count over a period of time. To identify persistent motion instead of a burst of motions, an input can include a count of consecutive time periods in which at least one large motion occurs.

As illustrated in FIG. 4B at 310, method 300 may further include automatically initiating electrical stimulation, via an electrode (e.g. implantable or external), to an upper airway patency-related nerve in response to detecting initial sleep onset or sleep onset after WASO. At 312, method 300 may further include automatically pausing electrical stimulation, via the electrode, in response to detecting WASO.

As illustrated in FIG. 4C at 314, method 300 may further include setting the first threshold values, the second threshold values, and the third threshold values based on a general population model. As illustrated in FIG. 4D at 316, method 300 may further include adjusting the first threshold values, the second threshold values, and the third threshold values based on patient feedback. In some examples, the patient feedback may comprise at least one of a survey, manual control of the electrical stimulation, polysomnography, a home sleep test, a wearable device, or a sleep mat. As illustrated in FIG. 4E at 318, method 300 may further include applying different weight values to each of the plurality of inputs. The different weight values may be applied to each of the plurality of inputs so that each input is considered relatively more or relatively less in comparison to the other inputs when detecting initial sleep onset, wake after sleep onset (WASO), and sleep onset after WASO. In some such examples, the relative weight applied to an input may be based on a confidence factor in the accuracy, reliability, and/or other factor, etc. of the particular input generally and/or under specific circumstances.

In some examples, the available inputs may vary depending on whether the patient is sleeping at home or away (e.g. hotel), which may be sensed. In some such examples, the example method and/or device may first determine which inputs are available, and then select, and/or place more weight on, the currently available inputs having the highest confidence factors.

In one specific example of method 300 as described with reference to FIGS. 4A-4E, a patient may get in bed (or lie down) and the initial sleep onset detection is enabled. In some examples, the initial sleep onset detection may be enabled in response to detecting the patient's intent to sleep. An intent to sleep time may be based on an input from an internal sensor (e.g. accelerometer, gyroscope, microphone), an external sensor (e.g. accelerometer, light sensor, motion sensor, sleep mat, wearable device, pneumatic sensor, low power radar sensor, etc.), a remote device (e.g. 4030 of FIG. 34B or 4340 of FIG. 36), and/or a user interface (e.g. 4040 of FIG. 35). The patient reads in bed for 15 minutes, and their ambient movement (e.g. of the first subset of inputs) prevents the detection of sleep onset. The patient turns off the lights and starts to fall asleep. The decrease (e.g. below the corresponding first thresholds) in their heart rate variability (HRV) (e.g. of the first subset of inputs) and body temperature (e.g. of the first subset of inputs) eventually results in the detection of sleep onset indicating that the patient is asleep. In response to detecting sleep onset, stimulation may be activated and the wake after sleep onset (WASO) detection is enabled. The patient wakes up in the middle of the night due to noise/external circumstances, and tries to fall back asleep. Their heart rate variability is still fairly low, their body temperature is still low, so the initial sleep onset detection would still be detecting sleep. However, the patient's locomotor inactivity during sleep (LIDS) standard deviation (e.g. of the second subset of inputs) has increased (e.g. above a corresponding second threshold) during this period, which results in the detection of WASO. In response to detecting WASO, the stimulation may be paused until the LIDS standard deviation (e.g. of the third subset of inputs) has decreased again (e.g. below a corresponding third threshold) and sleep onset after WASO is detected. In response to detecting sleep onset after WASO, the stimulation may be resumed.

In another specific example of method 300 as described with reference to FIGS. 4A-4E, a patient may get in bed (or lie down) around 10 pm and the initial sleep onset detection is enabled. The patient reads in bed for 15 minutes, and their ambient movement prevents the detection of sleep onset. The patient turns off the lights and starts to fall asleep. The decrease in their heart rate variability (HRV) and body temperature eventually results in the detection of sleep onset indicating that the patient is asleep. In response to detecting sleep onset, stimulation may be activated and wake after sleep onset (WASO) detection based on a time-dependent threshold is enabled. The patient wakes up at 2 am due to noise/external circumstances, and tries to fall back asleep. Four hours have elapsed since getting into bed and the ambient light is low since it is before sunrise. These factors contribute to higher thresholds that leads to detection of sleep onset after WASO sooner than the detection of initial sleep onset when the patient got in bed at 10 pm.

In yet another specific example of method 300 as described with reference to FIGS. 4A-4E, a patient may get in bed (or lie down) around 1 am and the initial sleep onset detection is enabled. The patient reads in bed for 15 minutes, and their ambient movement prevents the detection of sleep onset. The patient turns off the lights and starts to fall asleep. The decrease in their heart rate variability (HRV) and body temperature eventually results in the detection of sleep onset indicating that the patient is asleep. In response to detecting sleep onset, stimulation may be activated and wake after sleep onset (WASO) detection based on a time-dependent threshold is enabled. The patient wakes up at 5 am due to noise/external circumstances, and tries to fall back asleep. Four hours have elapsed since getting into bed and the ambient light is high since it is close to sunrise. These factors contribute to lower thresholds that leads to detection of sleep onset after WASO similar to the detection of initial sleep onset when the patent got in bed at 1 am since the sunlight and patient circadian rhythm may make it harder to fall back asleep.

The patient circadian rhythm may include the patient's bedtime, sleep duration, and awakening time. The patient circadian rhythm may be used alone, or in combination with other inputs to determine a sleep-wake status (e.g. initial sleep onset, wake after sleep onset, and/or sleep onset after WASO), which may be used for activating and/or deactivating stimulation. In some examples, the patient circadian rhythm may be determined based on demographics (e.g. age, gender), medical history (e.g. insomnia, depression, etc.), questionnaires (e.g. about current sleep habits, work schedules, etc.), temperature, geolocation (e.g. sunrise/sunset), external sensors (e.g. in/out bedtime pattern, lighting, mobile device use), physiologic signals/information (e.g. heart rate variability), the previous night's sleep hours, working hours and activity level, food and drink intake/timing, and/or the unified model of performance (UMP).

The sensors used to determine the patient circadian rhythm may be part of a stimulation element (e.g. 117 of FIG. 1B), an internal sensor (e.g. 128 of FIG. 1B), or an external element (e.g. 171, 150 of FIG. 1B), such as a stimulation element remote, a mobile device, a wearable device (e.g. smartwatch), a device near the patient (e.g. sleep mat), etc.

The patient circadian rhythm may be used for initiating automatic sleep detection and/or automatically activating and/or deactivating stimulation. In some examples, a combination of manual control and automatic control based on the patient circadian rhythm may be used where: 1) patient manually activates stimulation, but stimulation is automatically deactivated based on a projected wake time, and/or 2) device automatically activates stimulation based on a projected sleep time, but the patient manually deactivates the stimulation. In some examples, the patient circadian rhythm may also be used to provide relaxation and/or bedtime suggestions to the patient.

Stimulation may be automatically activated and deactivated based on predicted circadian rhythm cycles determined by a model (e.g. a data model such as (but not limited to) a machine learning model) as described below with reference to FIGS. 41-42). The model may receive multiple inputs and predict sleep time and wake up time based on the multiple inputs. A generalized model may first be developed that performs acceptably on a general population of patients with the same inputs/outputs as described above. The model may then be personalized (e.g. calibrated) based on the individual patient, geography, and daily activity, etc. The patient may input their own information (e.g. demographics, sleep habits, social-economic, geographic) to the model via a user interface. External sensor data (e.g. accelerometer, mobile device, light sensor, GPS, pneumatic sensor, low power radar sensor, etc.) may also be used to provide input data to the model. In addition, internal sensors (e.g. accelerometer, gyroscope, microphone, etc.) of a stimulation element (e.g. pulse generator) may also be used to provide input data to the model.

The model may generate a prediction of the ideal time for sleeping and waking up. The model may be implemented on an implanted medical device, an external device near the patient, or externally in a cloud-based application. In some examples, the model may be accessed via an application programing interface (API). The model prediction may be used to make recommendations to the patient, incentivize the patient, educate the patient on their ideal bedtime and waking time via a user interface, provide diagnostic data for a physician via a user interface, serve as inputs into a sleep detection algorithm for adjusting (e.g. calibrating) detection thresholds, and/or for adjusting (e.g. calibrating) the activation and/or deactivation times of stimulation.

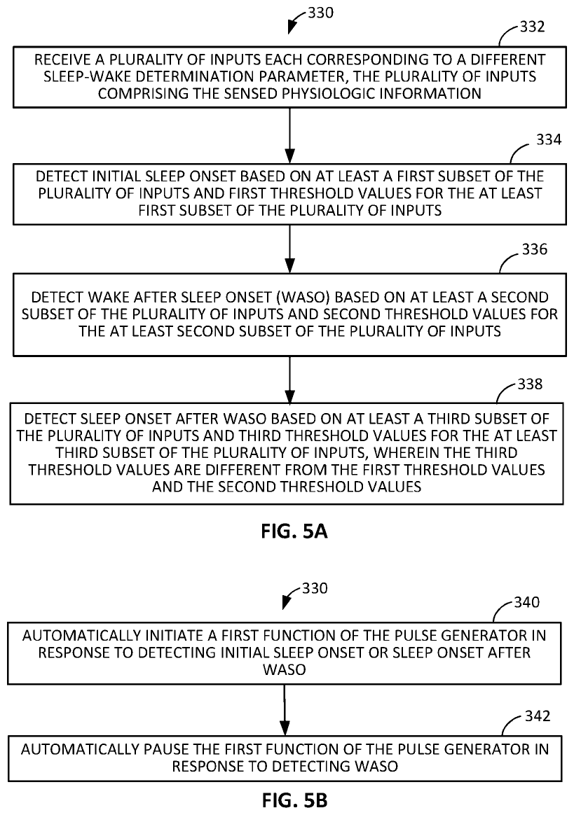

FIGS. 5A-5B are diagrams schematically representing another example method 330 for determining a sleep-wake status. Method 330 may be implemented by a medical device (e.g. an implantable medical device 117 of FIG. 1B) including at least one sensor (e.g. 128 of FIGS. 1B and/or 171, 150 of FIG. 1B) to sense physiologic information and a control portion (e.g. 190 of FIG. 1C). In some examples, the at least one sensor to sense physiologic information may comprise at least one of an accelerometer sensor or a temperature sensor. In some examples, the at least one sensor to sense physiologic information is implantable. In some examples, the at least one sensor to sense physiologic information is external to a patient's body. In some examples, the medical device further includes at least one sensor external to a patient's body to sense environmental information. The at least one sensor to sense environmental information may comprise at least one of a light sensor, a noise sensor, or a movement sensor.

As illustrated in FIG. 5A at 332, the control portion may receive a plurality of inputs each corresponding to a different sleep-wake determination parameter, the plurality of inputs comprising the sensed physiologic information. At 334, the control portion may detect initial sleep onset based on at least a first subset of the plurality of inputs and first threshold values for the at least first subset of the plurality of inputs. At 336, the control portion may detect wake after sleep onset (WASO) based on at least a second subset of the plurality of inputs and second threshold values for the at least second subset of the plurality of inputs. In some examples, the second subset of the plurality of inputs may be different from the first subset of the plurality of inputs. At 338, the control portion may detect sleep onset after WASO based on at least a third subset of the plurality of inputs and third threshold values for the at least third subset of the plurality of inputs, wherein the third threshold values are different from the first threshold values and the second threshold values.

In some examples, the medical device may further include a pulse generator (e.g. 117 of FIG. 1B) to apply electrical stimulation. In this case, as illustrated in FIG. 5B at 330, the control portion may further automatically initiate a first function of the pulse generator in response to detecting initial sleep onset or sleep onset after WASO. At 342, the control portion may further automatically pause the first function of the pulse generator in response to detecting WASO. In some examples, the pulse generator comprises an implantable pulse generator. In some examples, the first function of the pulse generator is to apply electrical stimulation to an upper airway patency-related nerve.

In some examples of method 330, the first threshold values and/or the third threshold values are time dependent. For example, during a sleep period, the third threshold values may be initially at minimum values at a start of the sleep period, increase from the minimum values to maximum values, and then decrease from the maximum values back to the minimum values by the end of the sleep period.

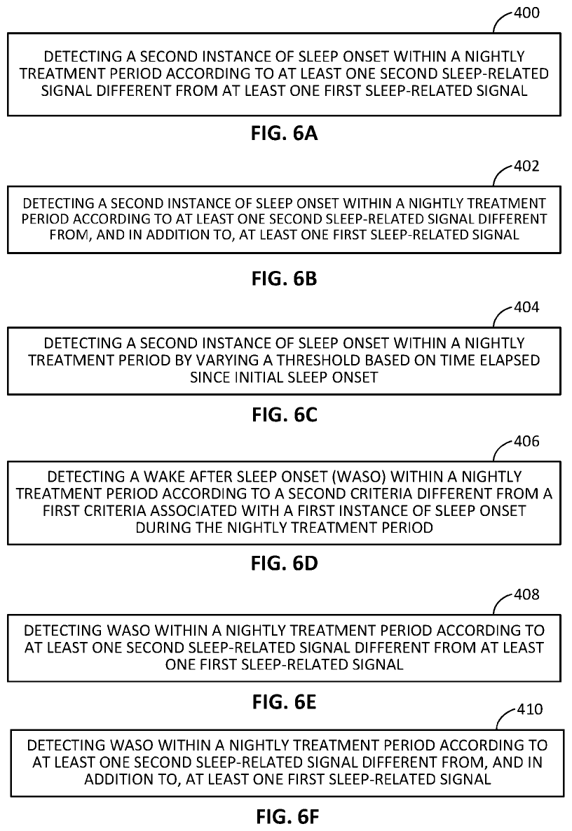

FIGS. 6A-6F are diagrams schematically representing yet other example methods for determining a sleep-wake status (e.g. initial sleep onset, wake after sleep onset, and/or sleep onset after WASO). The example methods of FIGS. 6A-6F may include at least some of substantially the same features and attributes as previously described with reference to FIGS. 2-5B. As illustrated in FIG. 6A at 400, a method may include detecting a second instance of sleep onset within a nightly treatment period according to at least one second sleep-related signal different from at least one first sleep-related signal. For example, the first sleep-related signal may comprise heart rate variability and the second sleep-related signal may comprise locomotor inactivity during sleep (LIDS).

As illustrated in FIG. 6B at 402, a method may include detecting a second instance of sleep onset within a nightly treatment period according to a least one second sleep-related signal different from, and in addition to, at least one first sleep-related signal.

As illustrated in FIG. 6C at 404, a method may include detecting a second instance of sleep onset within a nightly treatment period by varying a threshold based on time elapsed since initial sleep onset.

As illustrated in FIG. 6D at 406, a method may include detecting a wake after sleep onset (WASO) within a nightly treatment period according to a second criteria different from a first criteria associated with a first instance of sleep onset during the nightly treatment period.

As illustrated in FIG. 6E at 408, a method may include detecting WASO within a nightly treatment period according to at least one second sleep-related signal different from at least one first sleep-related signal.

As illustrated in FIG. 6F at 410, a method may include detecting WASO within a nightly treatment period according to at least one second sleep-related signal different from, and in addition to, at least one first sleep-related signal.

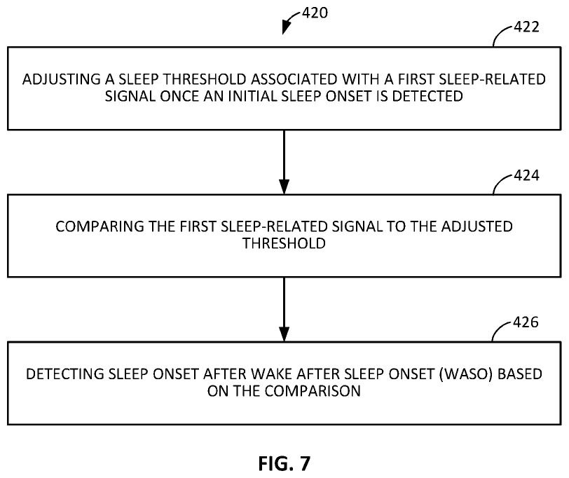

FIG. 7 is a diagram schematically representing yet another example method 420 for determining a sleep-wake status. At 422, method 420 includes adjusting a sleep threshold associated with a first sleep-related signal once an initial sleep onset is detected. At 424, method 420 includes comparing the first sleep-related signal to the adjusted threshold. At 426, method 420 includes detecting sleep onset after wake after sleep onset (WASO) based on the comparison.

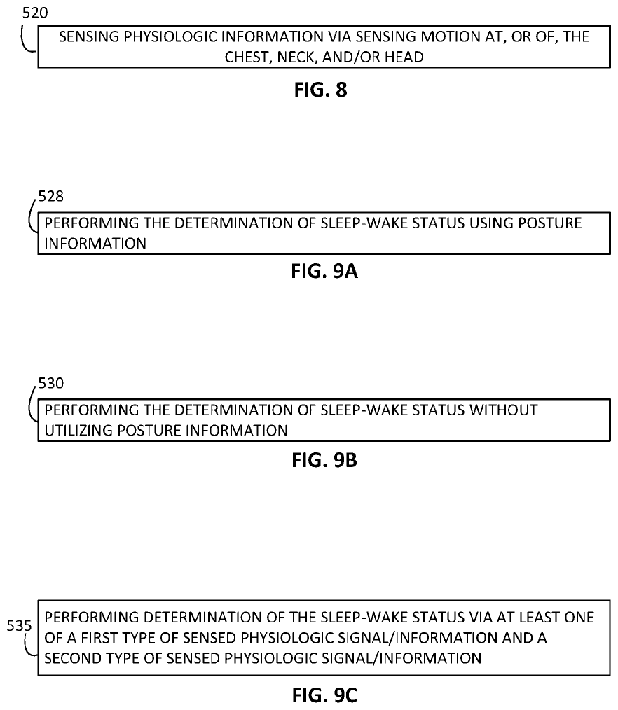

Various further aspects of determining a sleep-wake status (e.g. initial sleep onset, wake after sleep onset, and/or sleep onset after WASO) based on sleep-wake determination parameters and corresponding sleep-wake threshold values is further described below in association with at least FIGS. 8-42 of the present disclosure.

As schematically represented at 520 in FIG. 8, in some examples sensing the physiologic information may comprise sensing motion at, or of, the chest, neck, and/or head, which in turn may be used to determine the sleep-wake status. At least some aspects of such determination are further described in association with FIGS. 30A-32. For instance, sensing portion 2000 in FIG. 30A and/or care engine 2500 (including but not limited to sensing portion 2510) in FIG. 32 comprises multiple sensor types, modalities, etc., at least some of which may be used to sense motion at, or of, the chest, neck, and/or head, and to utilize such sensed motion to determine a sleep-wake status (e.g. detecting sleep). One such example modality may comprise employing an accelerometer to sense motion at the chest, neck, and/or head, as further described later. In some examples, the accelerometer may be implanted at the chest, neck, and/or head, while in some examples, the accelerometer may be secured externally on the patient's body at such locations or be present as part of a patient support, sleeping mat, wearable, etc. as described in association with at least FIG. 1B.

The sensed motion at the chest, neck, and/or head may comprise motion of the chest, neck, and/or head or may comprise motion phenomenon at those respective locations without necessarily involving gross motion of the chest, neck, and/or head, as further described later in association with at least FIGS. 31A-31F. In one non-limiting example, sensing the motion phenomenon at the neck or other location may comprise sensing circulation of blood within a blood vessel/vasculature (e.g. arterial motion within a vessel). In some such examples, the sensing element (e.g. accelerometer, impedance, other) may be at least partially incorporated in a microstimulator (or other implantable pulse generator) sized and shaped to be implantable within a blood vessel. The example method may comprise sensing ballistic motion of the blood vessel caused by the heartbeat of the patient. In some examples, the blood vessel may comprise an external jugular vein and hence the sensing of motion may occur at the neck in some examples without necessarily being motion of the neck (e.g. bending, titling, twisting, etc.).

As schematically represented at 528 in FIG. 9A, in some examples, a method of determining a sleep-wake status may be performed using sensed posture information and/or body position information. The sensed posture information may comprise a static posture or may comprise a change in posture, which may be considered a form of gross body motion mentioned above. As noted elsewhere, the sensed posture may be used to help confirm whether the patient is likely sleeping (e.g. lying down) or awake (e.g. sitting up) which may be in combination with other sensed information (e.g. heart rate, respiratory rate, etc.).

As schematically represented at 530 in FIG. 9B, in some examples, a method of determining a sleep-wake status may be performed without utilizing posture information and/or body position information.

For instance, a patient may sometimes intentionally (or unintentionally) sleep when sitting in a chair or an airline seat, and would benefit from SDB care (e.g. neurostimulation therapy). In such instances, determining sleep-wake status without using posture information may enhance quicker or more accurate detection of sleep for the patient sleeping in a sitting position because the example method may avoid a false negative indication (by a posture-based determination) that the patient is awake.

Conversely, a patient may sometimes intentionally be awake when lying horizontally, and accordingly, does not wish to receive SDB care. In such instances, determining sleep-wake status without using posture information may enhance quicker or more accurate detection of sleep for the patient who is awake in a lying-down position because the example method avoids a false positive indication (by a posture-based determination) that the patient is asleep because they are laying in the horizontal position typically associated with sleep.

As schematically represented at 535 in FIG. 9C, in some examples, a method of determining a sleep-wake status comprises sensing at least one of a first type of physiologic signal/information (e.g. respiratory signal, from which respiratory rate and/or other information may be derived and/or a cardiac signal, from which heart rate and/or other information may be derived) and a second type of physiologic signal/information (e.g. body movement), and performing determination of the sleep-wake status at least via at least one of the respective first type of sensed physiologic signal/information and the second type of sensed physiologic signal/information. In some examples, the sensed body movement may correspond to the sensed motion in FIG. 8. Various aspects of determination of the sleep-wake status based on such sensed physiologic information is further described in association with at least FIGS. 30A-32 and elsewhere throughout the various examples of the present disclosure.

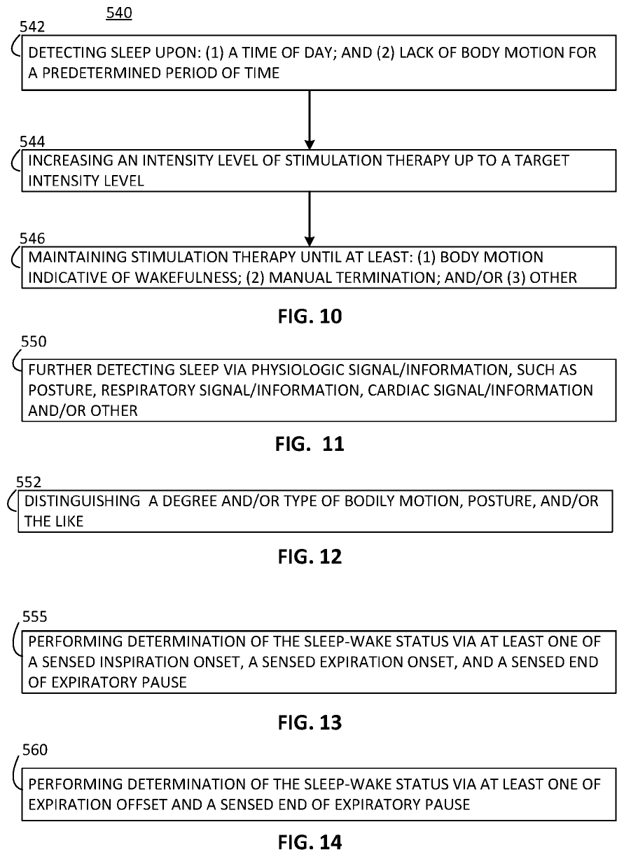

In some examples detecting sleep (and/or wakefulness) in association with delivering a stimulation therapy may comprise the method shown at 540 in FIG. 10. As shown at 542 in FIG. 10, the method 540 may comprise detecting sleep upon: (1) a time of day; and (2) detection of a lack of bodily motion indicative of sleep over a selectable, predetermined period of time. The time-of-day may be selectable and/or based on patient data. Once at least these two criteria are met, then as shown at 544 in FIG. 10, the method comprises increasing the intensity of the stimulation therapy from a lower initial intensity level to a target intensity level, such as in a ramped manner. As long as sensed physiologic information indicates that sleep is continuing, then stimulation at the target intensity level continues. However, upon the detection of body motion by the patient (which is indicative of wakefulness) or upon detection of the patient mechanically indicating wakefulness (e.g. physically tapping on chest near IPG), then the method may terminate any stimulation therapy and may remain in a no-stimulation mode for a selectable predetermined of time (e.g. 15 minutes). In other words, after the interruption, the method may delay therapy onset for a set period of time (e.g. 15 minutes). The length of the delay period is programmable.

As shown 550 in FIG. 11, in some examples the method 540 may further comprise sensing onset of sleep via additional physiologic signals/information, such as sensing posture, respiratory signals/information (e.g. stability based on respiratory period, depth, etc.), cardiac signals/information (e.g. stability based on per R-R interval, HR, etc.), and/or other information. For instance, in one non-limiting example, portion 542 of method 540 may comprise detecting posture (550) and comprise detection of sleep for some particular postures (but not others) and/or for some particular changes in posture (but not others). In some examples, the particular postures and/or particular changes in posture may be selectable by a patient and/or clinician. For example, the specified posture for which sleep is detectable may comprise a lying down posture (e.g. prone, supine, left side, right side) but the method not permitting auto-detection of sleep when a patient is sitting up.

In some instances, the example methods may detect (e.g. recognize) REM sleep and thereby avoid a false positive detection of wakefulness. In particular, while respiration during REM sleep does not exhibit the same stability as in non-REM sleep, such sensed less-stable respiration may be confirmed as occurring during REM sleep (and not wakefulness) based upon the patient having been asleep for some extended period of time (e.g. passage through multiple sleep stages, S1-S4) and upon the patient exhibiting a lack of bodily motion (e.g. of the type of bodily motion one would observe in wakefulness).

Implementation of method 540 also may comprise enhancing sensitivity to and/or specificity regarding the physiologic phenomenon being sensed.

In some examples, the detection of sleep (e.g. at 542) in method 540 in FIG. 10 also may comprise distinguishing a degree and/or type of bodily motion, posture, and the like as shown at 552 in FIG. 12. This distinguishing may be performed in association with ramping up stimulation (e.g. at 544), ramping down stimulation, terminating stimulation (e.g. 546), etc. For instance, via aspect 552 of method 540, the method may distinguish voluntary bodily motion as opposed to the jostling of the patient caused by vehicle motion (e.g. airplane, car, etc.) or by a bed partner. In some such examples, upon detecting such jostling, the method 540 may comprise temporarily decreasing stimulation therapy or pausing therapy, and then resuming the method at 544 to cause a quick return to target (e.g. therapeutic) intensity stimulation levels. In contrast, via aspect 552, the method 540 may identify physical tapping of the chest (near the IPG) as a voluntary bodily motion/cause or may identify a significant change to posture (e.g. change from lying down to sitting up) as being voluntary (e.g. not inadvertent) and then terminating therapy (or causing a longer pause) as at 546 in FIG. 10 because such detected behavior is indicative of wakefulness, whether temporary or longer term.

At least FIGS. 42-45 provide at least some example methods by which the determination of sleep-wake status may be made according to respiratory morphologic features. Moreover, at least some aspects of such sensing and related determination (of the sleep-wake status) relating to respiratory morphology features are further described in association with at least FIGS. 58 and 61A.

In one aspect, the various features of respiration morphologies addressed below in FIGS. 13-16 (e.g. inspiration onset, inspiration offset, magnitude, etc.) may enhance determining the sleep-wake status (e.g. at least sleep detection). In one aspect, these features of the respiratory morphology are readily identifiable and therefore beneficial to use in tracking a respiratory rate, which may be indicative of sleep (vs. wakefulness) according to the value of the respiratory rate, trend, and/or variability of the respiratory rate. In some examples, at least some of these features of respiration morphology may exhibit stability, which may be characteristic of sleep (vs. wakefulness). Some examples of such stability, which may be used to detect sleep/wake transitions, may include a stable respiratory rate, stability in an amplitude of the respiratory signal, stability of the percentage of the respiratory period corresponding to inspiration, and/or stability of the percentage of the respiratory period corresponding to expiration.

As schematically represented at 555 in FIG. 13, in some example methods, determining a sleep-wake status, such as via tracking at least some of the above-identified respiratory rate information, may comprise sensing at least one of an inspiration onset(s), an expiration onset(s), and end of expiratory pause, and performing determination of the sleep-wake status at least via at least one of the sensed inspiration onset(s), sensed expiration onset(s), and sensed end of expiratory pause.

As schematically represented at 560 in FIG. 14, in some example methods, determining a sleep-wake status, such as via tracking at least some of the above-identified respiratory rate information, may comprise sensing at least one of an expiration offset(s) and an end of expiratory pause(s) and performing determination of the sleep-wake status via at least one of the sensed expiration offset(s) and end of expiratory pause(s).

It will be understood that other combinations may be employed such as combining different combinations of fiducials (e.g. inspiration onset, end of expiratory pause, etc.) from FIGS. 13-16 or using just one of these fiducials from FIGS. 13-16 in determining a sleep wake status.

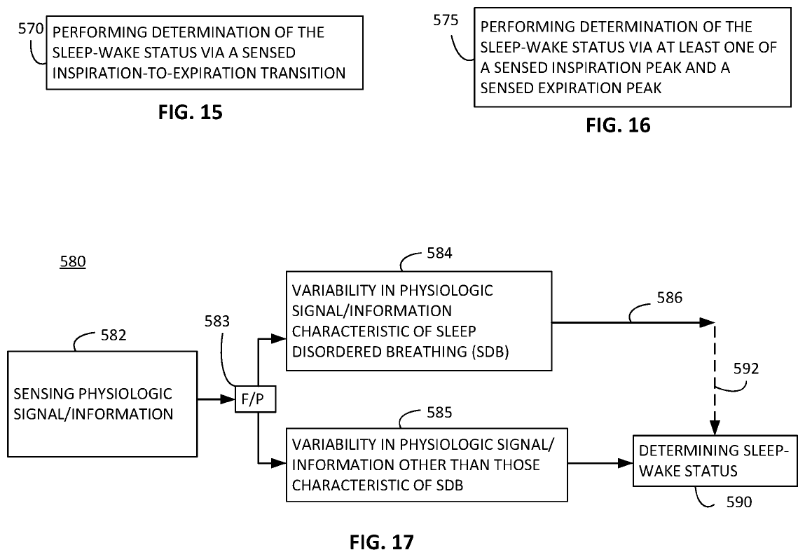

As schematically represented at 570 in FIG. 15, in some example methods, determining a sleep-wake status (such as via tracking at least some of the above-identified respiratory rate information) may comprise sensing an inspiration-to-expiration transition(s), and performing determination of the sleep-wake status at least via the sensed inspiration-to-expiration transition(s). Conversely, in some examples, sensing the physiologic information comprises sensing an expiration-to-inspiration transition(s), and determination of the sleep-wake status is performed via the sensed expiration-to-inspiration transition(s).

As schematically represented at 580 in FIG. 17, in some example methods, determining a sleep-wake status (such as via tracking at least some of the above-identified respiratory rate information) may comprise sensing at least one of an inspiration peak(s) and an expiration peak(s), and performing determination of the sleep-wake status via at least one of the sensed inspiration peak(s) and sensed expiration peak(s).

In some examples, at least some of the sensing of respiratory features, morphologies, etc. may be detected via sensing bioimpedance, as further described later in association with at least impedance parameter 2536 in FIG. 32. Of course, as noted elsewhere, sensing of such respiratory features, etc. may be implemented via sensing modalities other than, or in addition to, sensing bioimpedance. For instance, in some examples, at least some of the sensing of respiratory features, morphologies, etc. may be detected via sensing an electrocardiogra information, as further described later in association with at least ECG parameter 2520 in FIGS. 32 and/or 2020 in FIG. 30A.

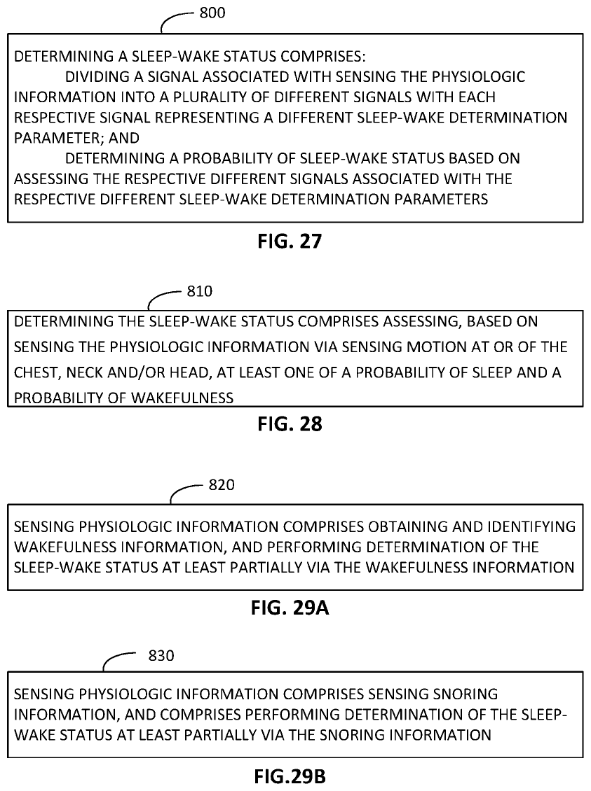

In some such examples associated with at least FIGS. 13-16 and/or at least FIGS. 27-30A, a method and/or device for determination of sleep-wake status via sensing variability in respiratory behavior, cardiac behavior, and/or other physiologic information may comprise identifying some features of such variability which are indicative of sleep disordered breathing (SDB) and differentiating the identified SDB-indicative features from other features of respiratory behavior, cardiac behavior, and/or other physiologic information, such as those which are indicative of a sleep or wakefulness.

For instance, as schematically represented in the block diagram of FIG. 17, in some examples a method 580 (or device for) determining a sleep-wake status may comprise sensing physiologic signals/information (e.g. respiratory features and/or cardiac features) as shown at 582. At 583, method 580 may comprise applying filtering and processing (F/P) of the sensed physiologic signals/information to produce: (1) filtered/processed physiologic signal information at 584 comprising variability in physiologic signals/information (e.g. respiratory features and/or cardiac features) which are characteristic of sleep disordered breathing (SDB); and (2) filtered/processed signal information at 585 comprising variability in physiologic signals/information (e.g. respiratory features and/or cardiac features) other than those characteristic of sleep disordered breathing (SDB). Via the signal information at 585, at 590 the method may comprise determining sleep-wake status. In some such examples, the sleep-wake status determination may comprise at least some of substantially the same features as described in association with at least FIGS. 13-16 or other examples described throughout the present disclosure.

With further reference to FIG. 17, in some examples, the output 586 of the information 584 may be used in monitoring, diagnosing, treating, etc. sleep disordered breathing (SDB). However, in some examples, via path 592 this sensed physiologic signal/information (e.g. respiratory and/or cardiac information) characteristic of sleep disordered breathing (SDB) may be used to confirm determination of a sleep-wake status, such as confirming that the patient is in a sleep state by confirming the occurrence of sleep disordered breathing. In making this confirmation, the method may identify characteristics of sleep disordered breathing including (but not limited to) at least some of the periodic nature of SDB, such as the reoccurring sequence of a flow limitation, an apnea (or hypopnea), and recovery. This identification also may comprise identifying similar periodic changes in heart rate occurring without detecting any gross changes in posture.

Alternatively, the method may comprise at least partially confirming that the patient is in a wake state (which is primarily determined by other information) via confirming the absence of sleep disordered breathing, such as due to the periodic nature of changes to respiratory patterns and heart rate without gross posture changes.

In some examples, determination of a sleep-wake status may be performed via sensed cardiac morphological features. At least some aspects of such sensing and related determination (of the sleep-wake status) relating to cardiac morphology features are further described in association with at least FIGS. 30A and 32.

Various features of cardiac morphologies may enhance determining the sleep-wake status (e.g. at least sleep detection) at least because these features of the cardiac morphology are readily identifiable and therefore beneficial to use in tracking a heart rate, which may be indicative of sleep (vs. wakefulness) according to value of, trend of, and/or the variability of the heart rate (HRV). In some examples, at least some of these features of cardiac morphology may exhibit increasing stability, which may be characteristic of sleep (vs. wakefulness). In some examples, at least some sleep stages may exhibit more or less variability in heart rate variability (HRV) and/or more or less variability in respiratory features, as noted above. For instance, more variability in cardiac features (e.g. heart rate, etc.) and respiratory features (e.g. respiratory rate, etc.) can be expected in REM sleep. At least some examples of determining a sleep-wake status may identify such variability in cardiac and respiratory signals characteristic of a REM sleep stage in a manner which can be distinguished from variability (or lack thereof in some instances) of cardiac and respiratory signals characteristic of wakefulness. For instance, when the sensing of a moderate increase in variability of respiratory and/or cardiac features follows other sleep stages (e.g. S3, S4) coupled with sensing a lack of body motion, then the example methods may identify that the patient in in REM sleep.

In some examples, at least some of the sensing of cardiac features, morphologies, etc. may be detected via sensing bioimpedance, as further described later in association with at least impedance parameter 2036 in FIGS. 30A and 2536 in FIG. 32. In some examples, at least some of the sensing of cardiac features, morphologies, etc. may be detected via sensing an electrocardiogra information, as further described later in association with at least ECG parameter 2020, 2520 in FIGS. 30A and 32, respectively. The bioimpedance and/or ECG which is used to sense cardiac features, morphologies, etc., also may be used to sense respiratory features, morphologies, etc. (as previously noted), or may be used to sense both cardiac and respiratory features, morphologies, etc. It will be further understood that FIGS. 30A, 32 provide additional example sensing types, modalities, etc. by which cardiac information (including but not limited to heart rate and/or heart rate variability) may be sensed, and which then may be used in determining sleep-wake status.

At least some of these relationships in cardiac morphology are further described in association with cardiac portion 2600 of care engine 2500 in FIG. 32.

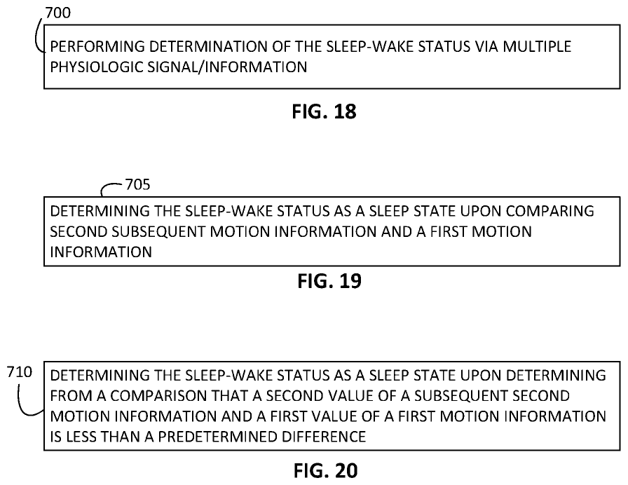

As schematically represented at 700 in FIG. 18, in some example methods, determining a sleep-wake status may comprise sensing multiple physiologic signals/information (e.g. at least respiration information, cardiac information (e.g. heart motion)), and performing determination of the sleep-wake status via the multiple physiologic signals/information (e.g. sensed respiration information and/or sensed cardiac information).

As schematically represented at 705 in FIG. 19, in some example methods, determining a sleep-wake status (e.g. onset of sleep, etc.) may comprise comparing subsequent second motion information to first motion information. As further shown at 710 in FIG. 20, in some examples, method 705 may comprise determining the sleep-wake status (e.g. onset of sleep, etc.) upon determining from the comparison that a second value of the subsequent second motion information and a first value of the first motion information is less than a predetermined difference. The value of the predetermined difference may be selectable.

In some examples of methods 705, 710, each of the respective first and second motion information comprises at least one of sensed respiratory information, sensed cardiac information, and sensed body motion.

In some examples of methods 705, 710, the subsequent second information comprises information obtained in the most recent sensed respiratory cycle and the first information comprises information obtained in a prior respiratory cycle. In some examples, the subsequent second information comprises information for respiratory activity in at least the last 30 seconds. In some examples, this information may relate to respiratory activity in at least the last 60 seconds. In some examples, this information may relate to respiratory activity in at least the last 7 breaths.

In some examples, the prior respiratory cycle comprises a respiratory cycle immediately preceding the most recent sensed respiratory cycle. In some examples, the prior respiratory cycle(s) comprise respiratory activity in the 30 seconds (or 60 seconds, or 7 breaths) preceding the most recently sensed respiratory cycle. In some examples, the first information comprises respiratory information over at least one respiratory cycle or at least 30 seconds or at least 60 seconds.

In some examples of methods 705, 710, recent motion information is compared to objective values indicative of sleep. In some examples, lower and/or more stable respiration rates and heart rates are more likely to be associated with sleep. As previously noted in association with at least FIG. 17, determining a sleep-wake status may comprise separating out (e.g. filtering, rejection) of respiratory features characteristic of sleep disordered breathing (SDB) and/or of respiratory features characteristic of particular sleep stages which do not necessarily contribute to general sleep detection (e.g. detecting onset of sleep).

However, as previously noted with respect to at least aspects 584, 592 in the method of FIG. 17, the detection of sleep disordered breathing (SDB) also may be used to sense or confirm the presence of sleep, or may be used to sense or confirm the onset of sleep in some instances.

In some examples, the method (at 705, 710 in FIGS. 19-20) may comprise determining the subsequent second motion information from a second average value of motion information in the respiratory cycles of the sensed second respiratory period and determining the first motion information from a first average value of motion information in the respiratory cycles of the first respiratory period. In some such examples, the second average value of motion information corresponds to an average of a parameter, such as but not limited to: an average amplitude of the sensed second respiratory period; an average respiratory rate of the sensed second respiratory period; and/or an average ratio of an inspiratory period relative to an expiratory period for the sensed second respiratory period.

It will be further understood that in some examples, the example implementations associated with FIGS. 19-20 may be used for any physiologic (e.g. biologic) signal of interest which may contribute to determining sleep-wake status throughout the various examples of the present disclosure.

In some examples, at least some of the aspects described above with respect to FIGS. 19-20 may be implemented via a history parameter 2542 and/or comparison parameter in sensing portion 2510 of care engine 2500, as later described in association with at least FIG. 32.

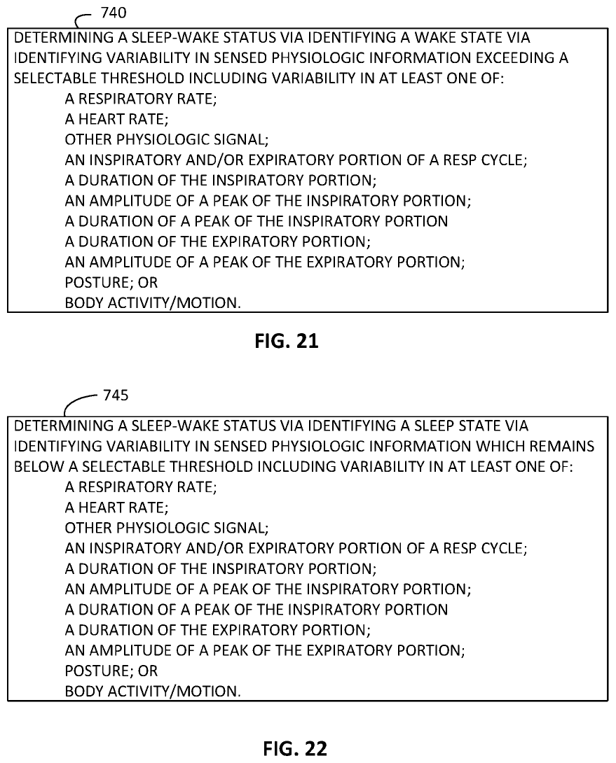

As schematically represented at 740 in FIG. 21, in some example methods, determining a sleep-wake status may comprise identifying a wakefulness state (or lack thereof) via identifying variability in sensed physiologic information including variability in at least one of: a respiratory signal and/or information derived therefrom (e.g. a respiratory rate); a cardiac signal and/or information derived therefrom (e.g. a heart rate); other physiologic signal (e.g. EEG, ECG, EMG, EOG, etc.) an inspiratory and/or expiratory portion of a respiratory cycle; a duration of the inspiratory portion; an amplitude of a peak of the inspiratory portion; a duration of a peak of the inspiratory portion; a duration of the expiratory portion; posture; body activity/motion; and an amplitude of a peak of the expiratory portion. In some examples, for at least some parameters, the variability may be evaluated relative to a threshold, which may be fixed in some examples. For example, determining a sleep-wake status may comprise identifying a wake state (or lack thereof) via identifying variability in sensed physiologic information exceeding a selectable threshold.

As schematically represented at 745 in FIG. 22, in some example methods, determining a sleep-wake status may comprise identifying a sleep state (or lack thereof) via identifying variability in sensed physiologic information including variability in at least one of: a respiratory signal and/or information derived therefrom (e.g. a respiratory rate); a cardiac signal and/or information derived therefrom (e.g. a heart rate); other physiologic signal (e.g. EEG, ECG, EMG, EOG, etc.) an inspiratory and/or expiratory portion of a respiratory cycle; a duration of the inspiratory portion; an amplitude of a peak of the inspiratory portion; a duration of a peak of the inspiratory portion; a duration of the expiratory portion; posture; body activity/motion; and an amplitude of a peak of the expiratory portion. In some examples, for at least some parameters, the variability may be evaluated relative to a threshold, which may be fixed in some examples. For example, determining a sleep-wake status may comprise identifying a sleep state (or lack thereof) via identifying variability in sensed physiologic information which remains below a selectable threshold.

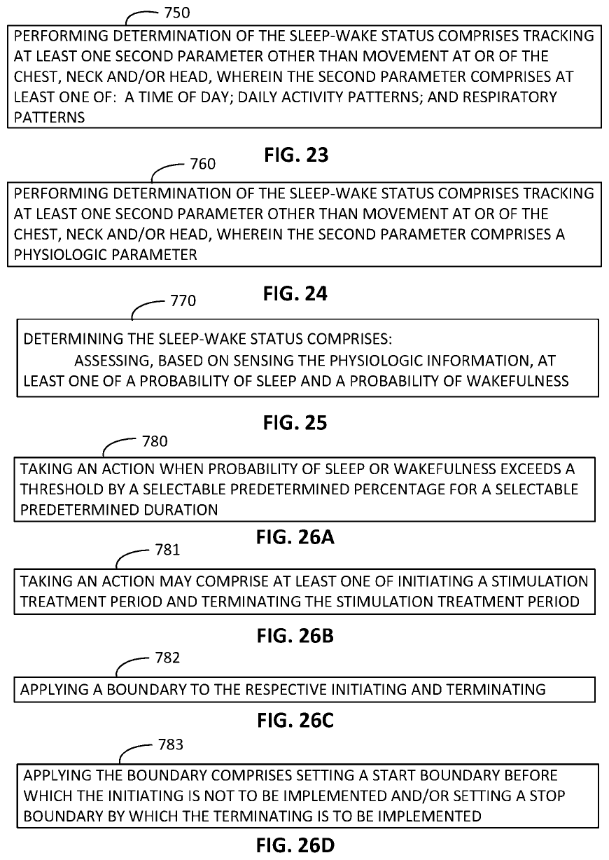

As schematically represented at 750 in FIG. 23, in some examples, performing determination of the sleep-wake status comprises tracking at least one second parameter other than movement at (or of) the chest, neck, and/or head, wherein the second parameter comprises at least one of: a time of day; daily activity patterns; and (typical) respiratory patterns.

As schematically represented at 760 in FIG. 24, in some examples, performing determination of the sleep-wake status comprises tracking at least one second parameter other than movement at (or of) the chest, neck, and/or head, wherein the second parameter comprises a physiologic parameter. For example, one such physiological parameter may comprise temperature (e.g. 2038 in FIGS. 30A, 2538 in FIG. 32).

As schematically represented at 770 in FIG. 25, in some examples, determining the sleep-wake status comprises assessing, based on sensing the physiologic information, at least one of a probability of sleep and a probability of wakefulness.

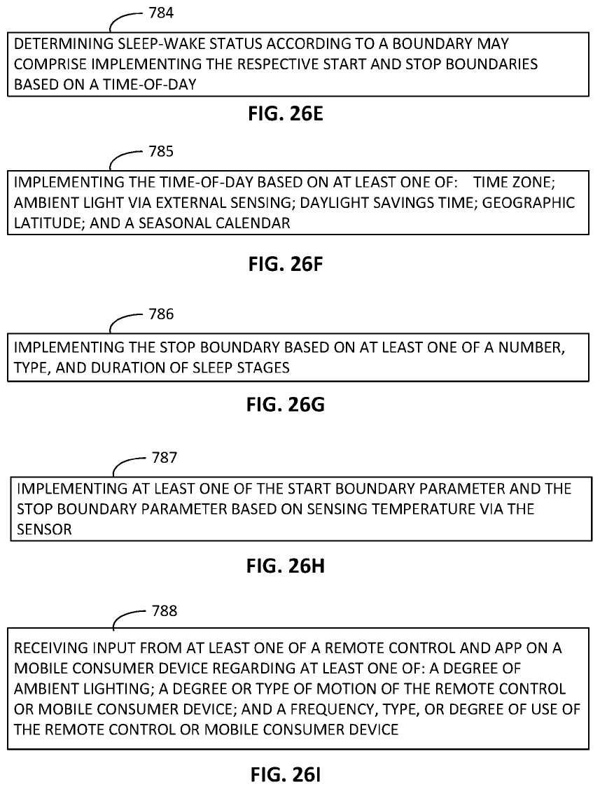

As schematically represented at 780 in FIG. 26A, some example methods (and/or devices) comprise taking an action when a probability of sleep or a probability of wakefulness exceeds a threshold. In some such examples, some example methods (and/or devices) comprise taking an action when a probability of sleep or a probability of wakefulness exceeds a threshold by a selectable predetermined percentage for a selectable predetermined duration.

In some examples of method 780 (FIG. 26A), taking an action may comprise at least one of initiating a stimulation treatment period and terminating the stimulation treatment period as shown at 781 in FIG. 26B. In some such examples, the taking an action (when a probability of sleep exceeds the threshold as in 780 in FIG. 26A) may comprise initiating a therapy treatment period (e.g. applying stimulation), resuming stimulation within a treatment period after a pause or suspension of stimulation, and/or other actions. In some examples, taking an action (when a probability of wakefulness exceeds the threshold) may comprise terminating a therapy treatment period, suspending stimulation within a treatment period, and/or other actions.

In some such examples, the initiating and/or resuming stimulation therapy may comprise employing a stimulation ramp in which an initial stimulation intensity is lower and then increased to a target intensity level. In some examples, terminating therapy may comprise employing a stimulation ramp in which a stimulation intensity is decreased gradually from a target therapy intensity level until stimulation is no longer applied (i.e. stimulation intensity equals zero).

With further reference to at least FIG. 26A, in some examples taking an action in method 780 may comprise use of an observer for an additional period of time to ensure the patient is asleep and/or using a start timer to initiate counting a selectable, predetermined period of time (e.g. delay) until stimulation is initiated as part of a treatment period.

In some such examples as method 780 (FIG. 26A), the method further comprises applying a boundary to the respective initiating and terminating as shown at 782 in FIG. 26C. At least some aspects of such a boundary are further described in association with boundary parameter 3016 of activation portion 3000 in FIG. 32.

In some example methods associated with method 780, applying the boundary comprises setting a start boundary before which the initiating is not to be implemented and/or setting a stop boundary by which the terminating is to be implemented, as shown at 783 in FIG. 26D.

In some examples, the method (e.g. 782, 783) of determining sleep-wake status according to a boundary may comprise implementing the respective start and stop boundaries based on a time-of-day, as shown at 784 in FIG. 26E. In some examples, as shown at 785 in FIG. 26F, the method may comprise implementing the time-of-day based on at least one of: time zone; ambient light via external sensing; daylight savings time; geographic latitude; and a seasonal calendar.

In some examples, as shown at 786 in FIG. 26G, the method (e.g. 783) may comprise implementing the stop boundary based on at least one of a number, type, and duration of sleep stages.

In some examples, as shown at 787 in FIG. 26H, the method (e.g. 783) may comprise implementing at least one of the start boundary parameter and the stop boundary parameter based on sensing temperature via the sensor (e.g. implantable, in some examples). In some examples, the method may comprise implementing, at least one of the initiating of the stimulation treatment period and the terminating of the stimulation treatment period, based on sensing body temperature via the sensor. In some examples, the method may comprise arranging the sensor within an implantable pulse generator and the sensor comprises a temperature sensor. In some such examples, method 787 may be implemented via, and/or is further described later in association with, at least temperature sensor 2038 in FIG. 30A, temperature parameter 2538 in FIG. 32, and/or at least boundary parameter 3016 in FIG. 32.

In some examples, as shown at 788 in FIG. 26U, the method (including determining the sleep-wake status such as at 770 in FIG. 25 and/or in 780 at FIG. 26A) may comprise receiving input from at least one of a remote control and app on a mobile consumer device regarding at least one of: a degree of ambient lighting; a degree or type of motion of the remote control or mobile consumer device; and a frequency, type, or degree of use of the remote control or mobile consumer device.

As schematically represented at 800 in FIG. 27, some examples of determining a sleep-wake status may comprise: dividing a signal associated with sensing the physiologic information into a plurality of different signals with each respective signal representing a different sleep-wake determination parameter; and determining a probability of sleep-wake status based on assessing the respective different signals associated with the respective different sleep-wake determination parameters. In some examples, this example method may comprise voting, by which each signal provides input to the overall probability of sleep. In some such examples, the various separate signals may be weighted differently so as to apply each respective sleep-wake determination parameter relatively more or relatively less in comparison to the other respective sleep-wake determination parameters.

In some examples, at least some aspects of method 800 (FIG. 27) may be implemented via at least some of the features and attributes of the arrangement described in association with at least FIGS. 30A-32.

As schematically represented at 810 in FIG. 28, in some examples determining the sleep-wake status comprises at least one of: assessing, based on sensing the physiologic information via sensing motion at (or of) the chest, neck, and/or head, at least one of a probability of sleep and a probability of wakefulness.

As schematically represented at 820 in FIG. 29A, in some examples, sensing physiologic information comprises obtaining and identifying wakefulness information (e.g. during normal wake periods), and comprising performing determination of the sleep-wake status at least partially via the wakefulness information. In some such examples, the wakefulness information is used to better characterize sleep and therefore more readily determine a sleep-wake status (e.g. such as detecting sleep or lack thereof). However, in this context, the identified wakefulness information is not used to adjust therapy (e.g. stimulation parameters, etc.) and/or not used to characterize a respiratory disorder. In some examples, the identification of wakefulness may be performed via sensing at least one of gross body motion and movement. In some examples, sensing physiologic information comprises obtaining sleep information, and comprising performing determination of the sleep-wake status via the sleep information.

As schematically represented at 830 in FIG. 29B, in some examples, a method comprises sensing snoring and using the snoring information as part of determining sleep-wake status. In some such examples, the method(s) may comprise quantifying the sensed snoring, and reporting snoring information to at least one of a patient, physician, or caregiver. In some examples, the snoring may be differentiated from normal speech. In some examples, snoring may be sensed, tracked, etc. in association with acoustic sensor 2039 (FIG. 30A) and/or acoustic parameter 2539 (FIG. 32).

In some examples, various features and attributes of the example methods (and/or care devices) described in association with at least FIGS. 1A-29B for determining sleep-wake status may be combined and implemented in a complementary or additive manner.

These, and additional features and attributes associated with FIGS. 1A-29B will be further described in association with at least FIGS. 30A-32. Moreover, at least some of the examples described in association with FIGS. 30A-42 may comprise example implementations of the examples described in association with FIGS. 1A-29B.

FIG. 30A is a block diagram schematically representing an example sensing portion. In some examples, an example method may employ and/or an example SDB care device may comprise the sensing portion 2000 to sense physiologic information and/or other information, with such sensed information relating to sleep-awake detection, among other uses. The sensed information may be used to implement at least some of the example methods and/or examples devices described in association with at least FIGS. 1A-29B and/or FIGS. 30B-42.

It will be understood that the sensing portion 2000 may be implemented as a single sensor or multiple sensors, and may comprise a single type of sensor or multiple types of sensing. In addition, it will be further understood that the various types of sensing schematically represented in FIG. 30A may correspond to a sensor and/or a sensing modality.

In some examples, the sensed information may refer to physiologic signals (e.g. biosignals) and/or metrics which may be derived from such physiologic signals. For instance, among other sensed physiologic information, one example of physiologic information may comprise respiration (2005) obtained from a respiratory signal and from which various metrics may be derived such as, but not limited to, respiratory rate, respiratory rate variability, respiratory phase, rate times volume, waveform morphology, and more. The respiration information and/or signal may be sensed via one or more sensing modalities described below (and/or other sensing modalities) such as, but not limited to, accelerometer 2026, ECG 2020, EMG 2022, ballistocardiogram 2023A, seismocardiogram 2023B, accelerocardiogram 2023C, impedance 2036, pressure 2037, temperature 2038, acoustic 2039, and/or other sensing modalities, at least some of which are further described below. In some examples, the sensed physiologic information may comprise cardiac information (2006) obtained from a cardiac signal and from which various metrics may be derived such as, but not limited to, heart rate (HR), heart rate variability (HRV), P-R intervals, waveform morphology, and more. One example of a cardiac signal may comprise an ECG signal, as represented at 2020 in FIG. 30A. Accordingly, the cardiac information and/or signal may be sensed via one or more sensing modalities further described below (and/or other sensing modalities) such as, but not limited to, accelerometer 2026, ECG 2020, EMG 2022, impedance 2036, pressure 2037, temperature 2038, and/or acoustic 2039. In some examples, the sensed physiologic information (e.g. via sensing portion 2000) may comprise a wide variety of physiologic information other than respiration and/or cardiac information, with at least some examples further described below in association with FIGS. 30A, 32, and other examples throughout the present disclosure.

The sensed physiologic signals and/or information (e.g. respiration 2005, cardiac 2006, and/or other information 2007) may be used for a wide variety of purposes such as, but not limited to, determining sleep-wake status (e.g. various sleep onset determinations), timing stimulation relative to respiration, determining disease burden, determining arousals, etc. In some such examples, the determination of disease burden may comprise detection of sleep disordered breathing events, which may be used in determining, assessing, etc. therapy outcomes such as, but not limited to, AHI, as well as titrating stimulation parameters, adjusting sensitivity of sensing the physiologic information, etc.

For instance, in one non-limiting example, an electrocardiogram (ECG) sensor 2020 in FIG. 30A may comprise a sensing element (e.g. electrode) or multiple sensing elements arranged relative to a patient's body (e.g. implanted in the transthoracic region) to obtain ECG information. In some examples, the ECG information may comprise one example implementation to obtain cardiac information, including but not limited to, heart rate and/or heart rate variability (HRV), which may be used (with or without other information) in determining sleep-wake status as described throughout the examples of the present disclosure.

However, in some instances, the ECG sensor 2020 may represent ECG sensing element(s) in general terms without regard to a particular manner in which sensing ECG information may be implemented.

In some examples in which multiple electrodes are employed to obtain an ECG signal, an ECG electrode may be mounted on or form at least part of a case (e.g. outer housing) of an implantable pulse generator (IPG), such as further described later in association with at least FIG. 31A. In such instances, other ECG electrodes are spaced apart from the ECG electrode associated with the IPG. In some examples, such as further described in association with FIG. 31A, at least some ECG sensing electrodes also may be employed to deliver stimulation to a nerve or muscle, such as but not limited to, an upper airway patency-related nerve (e.g. hypoglossal nerve) or other nerves or muscles.

In some examples, multiple ECG sensing electrodes may be mounted on or form different portions of a case of an IPG, such as later described in association with at least FIGS. 31B, 31C, 31D. In such examples, the respective ECG electrodes are arranged on the case of the IPG to be electrically independent of each other so that a suitable ECG signal may be obtained.

In some examples, an ECG sensing electrode may be used solely for sensing (e.g. single purpose) but is located along a lead body of a stimulation lead, as further described later in association with FIG. 31A. It will be understood that such dedicated ECG sensing electrode is positioned along the stimulation lead in a manner to avoid contact with a case of the IPG, particularly in examples in which an exposed electrically conductive portion of the case of the IPG may act as an electrode and by which a sensing vector may be obtained via a combination of the sensor electrode along the lead and the conductive portion of the IPG. Similarly, the same/similar electrode arrangement may be used to sense bioimpedance, as also described more fully later in association with FIGS. 31A-31F, 32.

In some examples, other types of sensing may be employed to obtain cardiac information (including but not limited to heart rate and/or heart rate variability), such as via ballistocardiogram sensor(s) 2023A, seismocardiogram sensor(s) 2023B, and/or accelerocardiogram sensor(s) 2023C as shown in FIG. 30A. In some examples, such sensing is based on and/or implemented via accelerometer-based sensing such as further described below in association with accelerometer 2026.

In one aspect, in some examples the ballistocardiogram sensor 2023A senses cardiac information caused by cardiac output, such as the forceful ejection of blood from the heart into the great arteries that occurs with each heartbeat. The sensed ballistocardiogram information may comprise heart rate (HR), heart rate variability (HRV), and/or additional cardiac morphology. In some examples such ballistocardiogram-type information may be sensed from within a blood vessel in which the sensor (e.g. accelerometer) senses the movement of the vessel wall caused by pulsations of blood moving through the vessel with each heartbeat. This phenomenon may sometimes be referred to as arterial motion.

In one aspect, the seismocardiogram sensor 2023B may provide cardiac information which is similar to that described for ballistocardiogram sensor 2023A, except for being obtained via sensing vibrations, per an accelerometer (e.g. single or multi-axis), in or along the chest wall caused by cardiac output. In particular, the seismocardiogram measures the compression waves generated by the heart (e.g. per heart wall motion and/or blood flow) during its movement and transmitted to the chest wall. Accordingly, the sensor 2023B may be placed in the chest wall.

In some such examples of sensing per sensors 2023A, 2023B, such methods and/or devices also may comprise sensing a respiratory rate and/or other respiratory information.

As further shown in FIG. 30A, in some examples the sensing portion 2000 may comprise an electroencephalography (EEG) sensor 2012 to obtain and track EEG information. In some examples, the EEG sensor 2012 may also sense and/or track central nervous system (CNS) information in addition to sensing EEG information. In some examples, the EEG sensor(s) 2012 may be implanted subdermally under the scalp or may be implanted in a head-neck region otherwise suitable to sense EEG information. Accordingly, the EEG sensor(s) 2012 are located near the brain and may detect frequencies associated with electrical brain activity.

In some examples, a sensing element used to sense EEG information is chronically implantable, such as in a subdermal location (e.g. subcutaneous location external to the cranium skull), rather than an intracranial position (e.g. interior to the cranium skull). In some examples, the EEG sensing element is placed and/or designed to sense EEG information without stimulating a vagus nerve at least because stimulating the vagal nerve may exacerbate sleep apnea, particularly with regard to obstructive sleep apnea. Similarly, the EEG sensing element may be used in a device in which a stimulation element delivers stimulation to a hypoglossal nerve or other upper airway patency nerve without stimulating the vagus nerve in order to avoid exacerbating the obstructive sleep apnea.

In some examples the sensing portion 2000 may comprise an electromyogram (EMG) sensor 2022 to obtain and track EMG information. In some such examples, the EMG sensor may comprise an electrode positioned near the tongue to detect signals indicative of voluntary control of the tongue, which in turn may be indicative of wakefulness. In some examples, the sensed EMG signals may be used to identify sleep and/or obstructive events. At least some additional aspects regarding EMG sensing are described in association with at least FIG. 31A.

In some examples, as shown in FIG. 30A, the sensing portion 2000 may comprise an EOG sensor 2024 to obtain and track EOG information, which may be used to a determine sleep-wake status and/or different sleep stages. In some instances, such sensed EOG information may be used to distinguish REM sleep from non-REM sleep or from wakefulness. In some examples, a sensing element for obtaining EOG information may be implanted in the head-and-neck portion, such as adjacent the eyes, eye muscles, and/or eye nerves, etc. In some examples, the sensing element may communicate the EOG information wirelessly, or via an implanted lead, to a control element (e.g. monitor, pulse generator, and the like) implanted within the head-and-neck region. In some such examples, the sensing element may comprise an electrode implanted near one or both eyes of the patient.

However, in some examples, the EOG information may be obtained via external sensing elements which are worn on the head or which may observe the eye movement, position, etc. such as via a mobile phone, monitoring station within proximity to the patient, and the like. Such externally-obtained EOG information may be communicated wirelessly to an implanted monitor, pulse generator and the like which controls sensing elements and/or stimulation elements implanted within the patient's body. Some aspects of sensing via EOG sensor are further described later in association with at least FIG. 32.

In some examples, any one or a combination of the various sensing modalities (e.g. EEG, EMG, etc.) described in association with FIG. 30A may be implemented via a single sensing element 2014.

In some examples, the sensing portion 2000 may comprise an accelerometer 2026. In some examples, the accelerometer 2026 and associated sensing (e.g. motion at (or of) the chest, neck, and/or head, respiratory, cardiac, posture, etc.) may be implemented according to at least some of substantially the same features and attributes as described in Dieken et al., ACCELEROMETER-BASED SENSING FOR SLEEP DISORDERED BREATHING (SDB) CARE, published as U.S. 2019-0160282 on May 30, 2019, and which is incorporated by reference herein in its entirety. In some examples, the accelerometer may comprise a single axis accelerometer while in some examples, the accelerometer may comprise a multiple axis (e.g. three axis) accelerometer. In some examples, a three axis accelerometer (e.g. x-axis, y-axis, and z-axis) may provide three sensor signals indicative of motion of a patient and the angle of the sensor relative to gravity.

Among other types and/or ways of sensing information, the accelerometer sensor(s) 2026 may be employed to sense or obtain a ballistocardiogram (2023A), a seismocardiogram (2023B), and/or an accelerocardiogram (2023C), which may be used to sense (at least) heart rate and/or heart rate variability (among other information such as respiratory rate in some instances), which may in turn be used as part of determining sleep-wake status as described throughout the examples of the present disclosure.

In some examples, the accelerometer 2026 may be used to sense activity, posture, and/or body position as part of determining a sleep-wake status, the sensed activity, posture, and/or body position may sometimes be at least partially indicative of a sleep-wake status.

In some examples, the sensing portion 2000 may comprise an impedance sensor 2036, which may sense transthoracic impedance or other bioimpedance of the patient. In some examples, the impedance sensor 2036 may comprise a plurality of sensing elements (e.g. electrodes) spaced apart from each other across a portion of the patient's body, such as electrodes 2120, 2135, 2130 in FIG. 31A, and/or example electrodes (e.g. 2310, 2402, 2404) in FIGS. 31B-31F. In some such examples, one of the sensing elements (e.g. electrode 2135 in FIG. 31A) may be mounted on or form part of an outer surface (e.g. case) of an implantable pulse generator (IPG) or other implantable sensing monitor, while other sensing elements (e.g. electrodes 2120, 2130 in FIG. 31A) may be located at a spaced distance from the sensing element of the IPG or sensing monitor. In at least some such examples, the impedance sensing arrangement integrates all the motion/change of the body (e.g. such as respiratory effort, cardiac motion, etc.) between the sense electrodes (including the case of the IPG when present). Some examples implementations of the impedance measurement circuit will include separate drive and measure electrodes to control for electrode to tissue access impedance at the driving nodes.

In some examples, the sensing portion 2000 may comprise a pressure sensor 2037, which senses respiratory information, such as but not limited to respiratory cyclical information. In some such examples, the respiratory pressure sensor may comprise at least some of substantially the same features and attributes as described in Ni et al., US Patent Publication US2011/0152706, METHOD AND APPARATUS FOR SENSING RESPIRATORY PRESSURE IN AN IMPLANTABLE STIMULATION SYSTEM, published on Jun. 23, 2011, and which is incorporated herein by reference in its entirety. In some examples, the pressure sensor 2037 may be located in direct or indirect continuity with respiratory organs or airway or tissues supporting the respiratory organs or airway in order to sense respiratory information.

In some examples, one sensing modality within sensing portion 2000 may be at least partially implemented via another sensing modality within sensing portion 2000.

In some examples, sensing portion 2000 may comprise an acoustic sensor 2039 to sense acoustic information, such as but not limited to cardiac information (including heart sounds), respiratory information, snoring, etc.

In some examples, sensing portion 2000 may comprise body motion parameter 2035 by which patient body motion (e.g. activity, locomotor inactivity during sleep) may be detected, tracked, etc. The body motion may be detected, tracked, etc. via a single type of sensor or via multiple types of sensing. For instance, in some examples, body motion may be sensed via accelerometer 2026 and in some examples, body motion may be sensed via EMG 2022 and/or other sensing modalities, as described throughout various examples of the present disclosure.

In some examples, the sensing portion 2000 in FIG. 30A may comprise a posture parameter 2040 to sense and/or track sensed information regarding posture, which also may comprise sensing of body position, activity, etc. of the patient. This sensed information may be indicative of an awake or sleep state of the patient in some examples. In some such examples, such information may be sensed via accelerometer 2026 as mentioned above, and/or other sensing modalities. In some examples, such posture information (and/or body position, activity) may be used sometimes alone and/or in combination with other sensing information to determine sleep-wake status. As described elsewhere herein, in some examples posture may be considered as one of several parameters when determining a probability of sleep (or awake).

For instance, sensing an upright posture typically is associated with a wakeful state, such as standing or walking. However, as noted elsewhere, a person could be in an upright sitting position and still be in a sleep state (e.g. sleeping in a chair). Accordingly, posture may be just one parameter used in determining a sleep-53 wake status, along with at least some other parameters described in association with sensing portion 2000 of FIG. 30A and/or care engine 2500 in FIG. 32. Conversely, sensing a supine or lateral decubitus (i.e. laying on a side) posture typically is associated with a sleep state. However, a patient might be in such a position without being asleep, such that other parameters (e.g. FIGS. 30A, 32) in addition to, or instead of, posture may significantly enhance determination of sleep-wake status.

Moreover, sensing posture may not be limited to sensing a static posture but extend to sensing simple changes in posture (or body position), which may be indicative of a sleep-wake status at least because certain changes in posture (e.g. from supine to upright) are mostly likely indicative of a wake state. Similarly, more complex or frequent changes in posture and/or body position may be further indicative of a wake state, whereas maintaining a single stable posture for an extended period of time may be indicative of a sleep state.

In some examples, the sensing portion 2000 comprises other sensor or parameter 2041 to direct sensing of, and/or receive, track, evaluate, etc. sensed information other than the previously described information sensed via the sensing portion 2000 (e.g. time, geolocation, proximity to sleeping area, light, noise, movement, etc.). In some examples, such sensed other information may be tracked, evaluated, etc. in association with other parameter 2541 in sensing portion 2510 of care engine 2500 in FIG. 32.

In some example methods and/or devices, via sensing portion 2000, a sleep-wake status may be determined without using posture information or body position information. In some such examples, a determination of a sleep-wake status without regard to posture information (or body position) may permit the device to provide efficacious sleep disordered breathing (SDB) care even when the patient may be sleeping in a vertical position, such as sitting in a chair, in a zero-gravity environment, etc. in contrast to a conventional assumption of sleep occurring in a horizontal body position. Such implementations may permit SDB care when a patient is sleeping during travel, such as sitting in an airplane seat, automobile seat, train seat, etc. In some such examples, a SDB care method and/or SDB care device may sometimes be referred to as being posture-insensitive.

As further shown in FIG. 30A, in some examples the sensing portion 2000 may comprise a temperature sensor 2038. In some examples, such sensed temperature may be tracked, evaluated, etc. in association with temperature parameter 2538 in sensing portion 2510 of care engine 2500 in FIG. 32.

In some examples, the sensed temperature may be used as one factor in making a sleep-wake status determination according to the examples of the present disclosure. In one aspect, the temperature sensor 2038 may sense and track a patient's normal fluctuation (e.g. temperature profile) in body temperature within a 24 hour daily period, which may exhibit on the order of a 2 degree F change. For most patients, their body temperature may reach and remain at the high end (e.g. 99.5 F) of its range during the middle of the day and evening (e.g. 7pm) before falling throughout late evening and overnight to the low end (e.g. 97.5 F) of its range by early morning (e.g. 5 or 6 am). In some examples, the temperature sensor 2038 may sense a change in the sensed temperature which occurs within a selectable time window of a 24 hour daily period and which exceeds a selectable threshold. In some examples, the selectable time window may comprise one hour, two hours, or other time periods. In some such examples, one method comprises selecting that a change of a predetermined number of degrees within the selectable time window will correspond to either a wake-to-sleep state transition or a sleep-to-wake state transition.

In some example methods, sensing a change in temperature (such as via sensor 2038) during a treatment period may be used to identify sleep disordered breathing behavior. In some such examples, additional sensed information (as described in examples of the present disclosure) may be used in addition to sensed temperature to identify sleep disordered breathing (SDB) behavior.

In some examples, this temperature fluctuation information sensed via temperature sensor 2038 may be used in association with boundary parameter 3016 (FIG. 32) to automatically implement a boundary or limit on the beginning and end of the treatment period, such that the lowest sensed body temperature may be used to at least partially mark a boundary of an end of a treatment period for a typical patient which sleeps at night. Similarly, the highest sensed body temperature (e.g. held for an extended period) may be used to at least partially implement a boundary at a beginning of a treatment period. In some such examples, these features may be used to implement method 787 in FIG. 26H.

In some examples, these same temperature-based boundaries may be used as one factor (among other factors) to determine a sleep-wake status. At least some other factors, which may be used with this sensed temperature fluctuation information to determine a sleep-wake status, may comprise a time of day parameter, accelerometer information, cardiac information, respiratory information, etc.

In some examples, smaller yet detectable temperature changes within a treatment period may be used to at least partially determine a sleep-wake status. For instance, a detectable temperature change may be sensed as a result of patient exertion to breathe in response to an apnea event, given the greater muscular effort in attempting to breathe.

Moreover, in some examples, such sensed temperature fluctuation information may provide a more distinctive or characteristic indication of a sleep or wake period when compared with heart rate or body position, which may exhibit more changes, some of which are not necessarily indicative of a sleep period or wake period, at least in some instances.

In some examples, at least some of the sensors and/or sensor modalities described in association with FIG. 30A (and/or FIG. 32) may be incorporated within or on a pulse generator (IPG 2133 in FIG. 31A), or within or on a microstimulator (e.g. FIGS. 31A-31F).

FIG. 30B is a block diagram schematically representing an example processing portion 2200, which may form part of and/or be in communication with at least sensing portion 2000 (FIG. 30A). In general terms, the processing portion processes signals and/or information obtained by a single sensor, single sensor type, or multiple types of sensors as described in association with at least FIG. 30A. As shown in FIG. 30B, processing portion 2200 may comprise a filtering function 2210 to filter the sensed signals to exclude noise, non-relevant information, etc. In some examples, the processing portion 2200 may comprise interpretation function 2212, which may interpret the information sensed via sensing portion 2000 in light of sensed physiologic information present in typical sleep patterns. In some such examples, the interpretation may be performed, at least partially with respect to information associated with a reference parameter 2220. In some such examples, the information available via reference parameter 2220 (for interpreting sensed information) may comprise a respiratory rate and/or respiratory signal morphology and/or may comprise a cardiac rate and cardiac signal morphology. Normalization may or may not be utilized.

In some examples, the sensing portion 2000 (FIG. 30A) and/or processing portion 2200 (FIG. 30B) may be employed in methods to extract important features from sensor signals. Such feature extraction may comprise band-pass filtering, frequency analysis, power spectral analysis, signal amplitude analysis, derivative signal analysis, use of thresholds, and differential signal analysis. Moreover, in some examples, such feature extraction also may comprise amplification and gain control, outlier rejection methods, be based on physiological rates, and/or wavelet analysis, as well as combinations of the preceding parameters. In some examples, the feature extraction may relate to and/or be performed to enable analysis of periods of periodic behavior. For instance, feature extraction may be performed on the sensed signal and the extracted feature may be analyzed as a moving average or in discrete time chunks as a distribution to determine if the particular extracted feature (e.g. heart rate, heart rate variability, respiratory rate, etc.) has reached a threshold of stability or exhibits a change from the previous behavior.

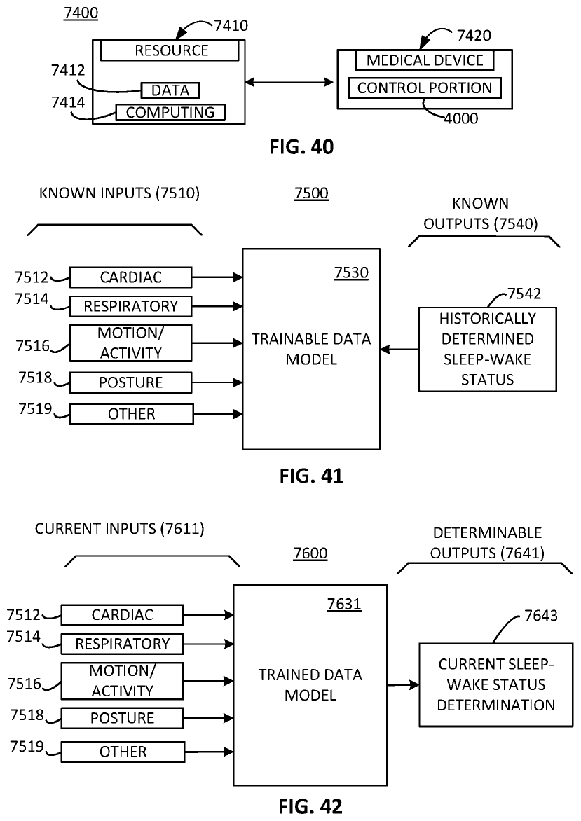

In some examples, at least a part of processing portion 2200 may comprise, and/or be implemented, by at least some of the features and attributes described in association with FIGS. 40-42.

In some examples, all or a portion of processing portion 2200 may be incorporated within sensing portion 2510 or other portions of care engine 2500 in FIG. 32 and/or may be incorporated within control portion 4000 (FIG. 34A).

FIGS. 30A-32 include diagrams schematically representing several example implementations of sensing elements, stimulation devices, related components, care engines, etc. for treating a patient, which may be employed in, may comprise an example implementation of, and/or may comprise at least some of substantially the same features and attributes as, of at least some of the example methods and/or example devices described throughout the present disclosure. In particular, in some examples, at least some aspects of the examples associated with FIGS. 30A-32 may comprise an example implementation of, may comprise at least some of substantially the same features and attributes as, and/or may include (or be exchanged with) additional/other elements from the various components, functions, and/or relationships of at least some of the examples previously described in association with at least FIGS. 1A-1C.

FIG. 31A is a diagram schematically representing several example implementations 2100 of sensing elements and a neurostimulation device 2113 implanted within a patient. As shown in FIG. 31A, the neurostimulation device 2113 may comprise an implantable pulse generator (IPG) 2133 and stimulation lead 2117, which comprises a lead body 2118 and a stimulation electrode 2112. The stimulation electrode 2112 is subcutaneously implanted and engaged relative to an upper airway patency-related nerve 2105, such as the hypoglossal nerve. In some examples, the IPG 2133 is implanted in the pectoral region 2101 with stimulation lead 2117 extending upward into the head-and-neck region 2103. In some examples, the stimulation electrode 2112 is chronically implantable, and may comprise a cylindrical arrangement to be at least partially wrapped about a nerve, may comprise a paddle-style electrode, may comprise a non-cuff configuration, or other configuration by which electrode may be chronically implanted in nerve-stimulating relation to the nerve.

In some examples, the stimulation electrode 2112 may comprise at least some of substantially the same features and attributes as described in Bonde et al. U.S. Pat. No. 8,340,785, SELF EXPANDING ELECTRODE CUFF, issued on Dec. 25, 2012 and Bonde et al. U.S. Pat. No. 9,227,053, SELF EXPANDING ELECTRODE CUFF, issued on Jan. 5, 2016, both which are hereby incorporated by reference in their entirety. In some examples, the stimulation electrode 2112 may comprise at least some of substantially the same features and attributes as described in Johnson, U.S. Pat. No. 8,934,992 NERVE CUFF, issued on Jan. 13, 2015, and/or in Rondoni, CUFF ELECTRODE, published as WO 2019/032890 on Feb. 14, 2019 (and filed as U.S. application Ser. No. 16/485,954 on Aug. 14, 2019), both which are hereby incorporated by reference in their entirety. Moreover, in some examples the stimulation lead 2117 may comprise at least some of substantially the same features and attributes as the stimulation lead described in U.S. Pat. No. 6,572,543 to Christopherson et al., and which is incorporated herein by reference.

However, it will be understood that in some examples the IPG 2133 also may take the form of a microstimulator, which is sized and placed, in the head-and-neck region 2103 in close proximity to the upper airway patency-related nerve 2105 to be stimulated. In some such examples, the microstimulator 2133 also may incorporate and/or include the stimulation electrode 2112 and/or sensing electrodes. In some example implementations in which the IPG 2133 may comprise a microstimulator, then placement of the microstimulator in the head-and-neck region 2103, such as in close proximity to the upper airway patency-related nerve would also place any exposed electrodes (e.g. 2135) on microstimulator in closer proximity to the nerve 2105, as well as in closer proximity to the head portion 2106 from which EEG information (including sleep information) may be determined via such electrode 2135. In some examples, such example microstimulators may comprise at least some of substantially the same features and attributes as described in association with at least MICROSTIMULATION SLEEP DISORDERED BREATHING (SDB) THERAPY DEVICE, published on May 26, 2017 as PCT Publication WO 2017/087681 from application PCT/US 2016/062546 filed on Nov. 17, 2016, and filed as U.S. application Ser. No. 15/774,471 on May 8, 2018, both of which are which is incorporated herein by reference. In such example arrangements including a microstimulator as the IPG 2133, the stimulation lead 2117 may be omitted (while still retaining stimulation electrode 2112) or the stimulation lead 2117 may be significantly shortened.

Via such neurostimulation device(s) (2133, 2112), delivery of a stimulation signal to the upper airway patency-related nerve 2105 may cause contraction of at least some upper airway patency muscles (e.g. the genioglossus muscle) to cause at least protrusion of the tongue to maintain or restore upper airway patency, and thereby provide therapeutic treatment of obstructive sleep apnea. At least some further example implementations regarding such stimulation are described in association with at least FIGS. 32-42.

In some examples, the example microstimulator may be implanted in the head-and-neck region (e.g. 2103) of a patient to sense at least some of the desired sleep-wake-related information, which may be used to perform sleep-wake determination. In some examples, sleep-wake determination may be used to implement, control, adjust, etc. therapy of sleep disordered breathing per neurostimulation of upper-airway-patency related nerves, muscles, tissues, etc. At least some example implementations of a head-and-neck implanted microstimulator may take the form described later in association with at least FIGS. 31B-31F.

In some examples, such as described later in FIGS. 31B-31F, the device implanted within the head-and-neck region may comprise a sensing element forming a part of and/or associated with the microstimulator. In some such examples, the sensing element(s) may be used to determine sleep-wake via detection of cardiac signals such as heart rate based on ECG or arterial motion. In some examples, the sensing element(s) may be used to determine sleep-wake via detection of respiratory signals such as respiratory motion or the subset of such motions that could be considered sounds including, but not limited to, snoring. In some such examples, the sensing element(s) may detect both cardiac signals and respiratory signals.

In some examples, whether involving microstimulation or involving other implantable pulse generators (IPG 2133 in FIG. 31A), changes to sensed signals after and/or during stimulation may be used to quantify therapy effectiveness and/or may be used to implement auto-titration of the stimulation, as further described later in association with at least FIG. 32.

In some examples, the stimulation electrode 2112 also may serve as a sensing element to sense physiologic information. In some such examples, the electrode 2112 may act as the sole sensing element to sense the physiologic information, such as a single channel EEG electrode or a single channel ECG electrode or other sensing modalities per sensing portion 2000 (FIG. 30A) or sensing portion 2510 (FIG. 32). As noted below, in some examples the stimulation electrode 2112 may be used for sensing in combination with other sensing elements and/or sensing modalities.

In some examples, the stimulation lead body 2118 may comprise a sensing element (e.g. electrode) 2120, which may act as the sole sensing element to sense the physiologic information, such as cardiac information, EEG information, EMG information, movement information, etc. in accordance with sensing portion 2000 (FIGS. 30A), 2510 (FIG. 32). Accordingly, in some examples, the sensing element 2120 may comprise an accelerometer. However, in some examples a sensing element (e.g. electrode) 2120 may be considered the sole sensing element when used in association an electrically conductive exterior portion (e.g. at least part of a case/housing) of an implantable stimulator (e.g. IPG or microstimulator).

In some examples, a single/sole sensor may comprise a pressure sensor (e.g. 2037 in FIG. 30A), and in some examples, pressure sensed via sensor 2037 may be tracked, evaluated, etc. via pressure parameter 2537 in sensing portion 2510 of care engine 2500 in FIG. 32.

In some examples, the EMG information sensed via one of the electrodes (e.g. 2120, 2112, etc.) may comprise detecting upper airway patency to assess obstruction (e.g. degree, location, etc.) and/or assess stimulation effectiveness, as well as detecting (and/or assessing) inhalation/exhalation during respiration. In some examples, the sensed EMG information may comprise sensing intercostal muscle activity in order to identify respiratory cyclical information (e.g. inspiration, expiration, expiratory pause) and/or identify or differentiate between central sleep apnea and obstructive sleep apnea.

However, in some examples, one or both of electrode 2112 and electrode 2120 may be used in association with other sensing elements (e.g. electrodes) to sense physiologic information.

In some examples, IPG 2133 comprises a sensing element(s) 2135. In some such examples, the sensing element 2135 is located on a surface of (or forms) a case of IPG 2133, and one of both of electrodes 2112 and 2120 may be used in association with electrode 2135 to measure bioimpedance (2036 in FIG. 30A; 2536 in FIG. 32), to obtain an ECG signal, an EMG signal, etc., and/or to sense cardiac information (including cardiac morphology), respiratory information (including respiratory morphology), and/or motion/movement of the chest, neck, and/or head, etc.

In some such examples, the sensing element 2135 of the IPG 2133 may comprise an accelerometer, which may comprise a single axis or multiple-axis (e.g. three axis) accelerometer. The accelerometer may be located internally within the IPG 2133, may be located externally on the IPG 2133, or may extend a short distance from the IPG 2133 via a small lead body.

As discussed in association with at least parameters 2026, 2526 in FIGS. 30A and 32, respectively, the accelerometer may be employed to sense motion at (or of) the chest, neck, and/or head, cardiac information, respiratory information, etc. In some examples, the accelerometer may be used to sense body activity/movement/motion, such as gross body motion (e.g. walking, talking), which may be indicative of activity associated with wakefulness. Alternatively, sensing a lack of activity via an accelerometer may be indicative of a sleep state, in some examples. In some such examples, the accelerometer may be used to sense physiologic information for use in at least some of the example methods of determining sleep-wake status without being used to sense posture or body position, as previously described herein. However, in some examples, the accelerometer may be used to sense such posture or body position.

With further reference to FIG. 31A, in some examples of determining a sleep-wake status, an electrode 2110 may be implanted subdermally in a head portion 2106 of a head-and-neck region 2103 (e.g. above the shoulder) to sense electrical brain activity and obtain EEG information and/or other central nervous system (CNS) information, with such sensed information being used to determine the sleep-wake status. Among other aspects, sleep onset, sleep termination, and/or various sleep stages may be determined via the sensed EEG information. In some examples, multiple electrodes 2110 may be placed subdermally about the head portion 2106 to sense such EEG information. In some examples, a single electrode 2110 may be used in combination with another electrode, such as the stimulation electrode 2112, to sense such EEG information. In some examples, the electrode 2110 may comprise the sole sensing element used to determine sleep-wake status.

In some example methods and/or devices of determining a sleep-wake status, an electrode 2114 may be implanted in or in close proximity to a tongue 2115 to sense electromyography (EMG) information. This sensed EMG information may include signals which are indicative of voluntary control of the tongue (e.g. talking, eating, etc.), which in turn may be indicative of wakefulness. In addition, this sensed EMG information may include signals which are indicative of sleep and/or sleep disordered breathing (e.g. obstructive events) such as when the tongue may relax into a position obstructing the upper airway.

It will be understood that just some of the various electrodes shown in FIG. 31A may be implanted or present in a particular example implementation. Moreover, while some of the electrodes (if present) may be used in combination with each other, some of the electrodes may be used to implement a particular sensing modality periodically or selectively rather than all of the time. For instance, there may be periods of time in which some electrodes are used to sense one modality (e.g. cardiac information, such as an ECG or other), while some electrodes are used to sense another modality (e.g. impedance) during some periods of time, with such periods of time being overlapping, coincidental, or independent of each other.

With this in mind, in some examples, one or multiple sensing modalities for determining wake-sleep status may be implemented in some instances while another, a different sensing modality (or a different combination of sensing modalities) may be implemented in other instances. For example, certain sensing modalities may be employed solely or less significantly during a portion of the daily period (e.g. normal wake period, such as 6 a.m. to 10 p.m.) and then not employed at all or less significantly during another portion of the daily period (e.g. normal sleep period), or vice versa.

In some example methods and/or devices, the normal wake period may be identified via at least one of clinician input, patient input, data model (e.g. machine learning, other), and other observational criteria. In the example of clinician input or patient input, a user may directly specify the start time and/or end time of the normal wake period (and conversely the normal sleep period). In some examples, the normal wake period (or conversely the normal sleep period) may be at least partially determined via historical data for a particular patient and/or historical data regarding multiple patients or the general population. In some such examples, a data model (e.g. data model parameter 3230 in FIG. 32) may be constructed, trained, etc. and applied to the historical data to make the determination. In some such examples, the data model may comprise data obtained, used, etc. on an on-going basis, such as on a daily basis, using at least the most recent historical data (e.g. last 30 days).

In some examples, as described later, a probability of sleep (or the sleep-wake status) may be determined from among a plurality of sleep-wake status parameters in which different sleep-wake status parameters may be weighted differently. Such different weighting for a given sleep-wake status parameter may depend on a time-of-day, clinician/patient input, etc.

As schematically represented in FIG. 31AA, in some examples the IPG 2133 of FIG. 31A may comprise a plurality of sensing elements (e.g. electrodes 2145, 2147) mounted on, or formed as part of, an outer surface (e.g. case) of the IPG 2133. As previously described elsewhere, this arrangement may be used to sense cardiac information (e.g. ECG, other), impedance, etc.

In some examples, whether mounted on a single housing (e.g. IPG 2133 in FIG. 31AA) or placed in multiple different locations (or on different components), the electrode(s) shown in FIGS. 31A, 31AA may be used to sense cardiac information (including cardiac morphology), respiratory information (including respiratory morphology), motion/movement of the chest and/or neck, etc., as described in association with sensing portion 2000 (FIG. 30A) and/or sensing portion 2510 (FIG. 32). In some examples, this sensed information may comprise a respiratory rate and/or a heart rate. In some such examples, the sensed respiratory information and/or cardiac information may comprise at least some of substantially the same features and attributes as described in association with respiration portion 2580 and/or cardiac portion 2600 in FIG. 32.

In some examples, the respiratory information is obtained via measuring trans-thoracic impedance solely via the electrodes on the IPG or via electrodes in addition to those present on the surface of the IPG. However, in some examples, the respiratory information may be derived from the ECG information.

With this in mind, in some examples described elsewhere in the present disclosure, the respiratory information and/or cardiac information may be obtained via an accelerometer (2026 in FIG. 30A), which may be located in the IPG 2133. As previously noted, there may be times at which the accelerometer is used to sense respiratory information, cardiac information, and/or other information in order to determine sleep-wake status while at other times, sensing modalities (e.g. ECG electrodes, EMG electrodes, etc.) other than an accelerometer may be used to sense respiration information, cardiac information, and/or other information to determine sleep-wake status.

Unless noted specifically otherwise, it will be understood that the electrodes described in FIG. 31A comprise an exposed electrically conductive portion to engage bodily tissues, etc. within the patient.

In some examples, a single SDB care device comprises a single housing. In some examples, the single device comprises an on-board power source. In some examples, a single device comprises a plurality of sensing elements (e.g. electrodes). In some examples, at least one sensing element (e.g. electrode) is located on two separate portions of a device. For instance, one electrode may be located on IPG 2133 while one electrode may be located on a stimulation lead body 2118.

As previously described in association with at least FIGS. 31A-31AA, in some examples an implantable pulse generator (IPG) may take the form of a microstimulator, and may be used to implement various sensing modalities as previously described. At least some example implementations of such a microstimulator are shown in at least FIGS. 31B and 31E.

As shown in the schematic representation 2300 in FIG. 31B, an example device 2359 including an example microstimulator 2355 may be implanted in a head-and-neck region 2302 of a patient, and in particular in the neck region 2303 in this example. The microstimulator 2355 is implanted subcutaneously via access-incision 2311 in area 2354. In the particular illustrated example, a stimulation electrode 2310 is electrically connected to and extends from the microstimulator 2355, with stimulation electrode 2310 coupled to nerve 2305 to stimulate the nerve, which causes contraction of musculature (e.g. tongue) to maintain or restore upper airway patency to treat sleep disordered breathing. In some examples, the stimulation electrode 2310 may comprise at least some of substantially the same features and attributes as stimulation electrode 2112 in FIG. 31A, including acting as a sensing electrode in some examples.

As further shown in the schematic representation of an example device 2400 in FIG. 31C, in some examples the microstimulator 2355 may comprise at least one electrode (e.g. 2402 and/or 2404) relative to which sensing vectors V1, V2, and/or V3 among electrodes 2310, 2402, 2404 may be established to sense physiologic phenomenon (e.g. ECG, bioimpedance, motion at (or of) the neck 2303, etc.) as previously described. This sensed physiologic information may be used to determine a sleep-wake status, among other things, such as implementing stimulation therapy. It will be further understood that in some examples, additional sensing modalities (e.g. EMG) described in association with FIGS. 30A, 31A, and 32 may be implemented via at least a portion of the microstimulation devices of FIGS. 31B-31F. While not fully shown in FIG. 31B, FIG. 31C illustrates that electrode 2310 may be arranged on a lead 2313 extending from microstimulator 2355.

As further shown in the schematic representation of an example device 2420 in FIG. 31D, in some examples the microstimulator 2355 may comprise an accelerometer 2422 by which sensing physiologic information (e.g. via sensing motion at or of the neck, etc.) may be implemented as previously described throughout the present disclosure. In addition, the microstimulator 2355 in FIG. 31D also may comprise an electrode 2402 (as in FIG. 31C) by which at least some of the previously described sensing (e.g. cardiac, ECG, bioimpedance, motion, etc.) may be implemented via sensing vector V2. This sensed physiologic information may be used to determine a sleep-wake status, among other things, such as implementing stimulation therapy.

FIG. 31E provides a schematic representation 2450 of an example device 2459 which comprises at least some of substantially the same features as the devices of FIGS. 31B-31D, except further comprising a dedicated sensing lead 2433 extending (subcutaneously) into tissue from microstimulator 2355 to support at least one electrode (e.g. 2431, 2432) spaced apart from microstimulator 2355 and/or other electrodes (e.g. 2431, 2432 or 2404 in FIG. 31F). This arrangement may be used to sense physiologic information (e.g. ECG, bioimpedance, motion at or of neck 2303) via vectors V1, V2, V5, V6, and/or V7 as illustrated in schematic representation 2460 of FIG. 31F in a manner similar to that described for at least the example arrangement of FIGS. 31B-31D.

In some examples, the microstimulator devices of FIGS. 31B-31F facilitate SDB care, including sleep-wake determination, in a compact arrangement in which sensing, stimulation, implant-access, etc. may be implemented in a single body region (e.g. neck) instead of being dispersed among several body regions (e.g. neck and torso), thereby simplifying implantation and SDB care. For instance, the neck-located microstimulator devices may sense physiologic phenomenon (e.g. respiration, cardiac, etc.) which may sometimes primarily be associated with a different region of the body (e.g. chest) while simultaneously conveniently placing a stimulation element in the neck region in which the microstimulator is located.

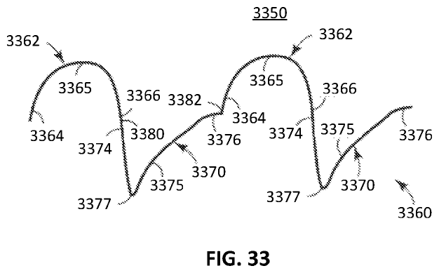

FIG. 32 is a block diagram schematically representing an example care engine 2500. In some examples, the care engine 2500 may form part of a control portion 4000, as later described in association with at least FIG. 34A, such as but not limited to comprising at least part of the instructions 4011 and/or information 4012. In some examples, the care engine 2500 may be used to implement at least some of the various example devices and/or example methods of the present disclosure as previously described in association with FIGS. 1A-31F and/or as later described in association with FIGS. 33-42. In some examples, the care engine 2500 (FIG. 32) and/or control portion 4000 (FIG. 34A) may form part of, and/or be in communication with, a pulse generator (e.g. 2133 in FIGS. 31A-31AA) whether such elements comprise a microstimulator or other arrangement.

In one aspect, at least the sensing portion 2510 of care engine 2500 in FIG. 32 directs the sensing of information, and/or receives, tracks, and/or evaluates sensed information obtained via one or more of the sensing modalities, sensing elements, etc. of sensing portion 2000 (FIG. 30A), with care engine 2500 employing such information to determine sleep-wake status, among other actions, functions, etc. as further described below.

As shown in FIG. 32, in some examples the care engine 2500 comprises a sensing portion 2510, a sleep state portion 2650, a sleep disordered breathing (SDB) parameters portion 2800, and/or a stimulation portion 2900. In some examples, the sensing portion 2510 may comprise an EEG parameter 2512 to sense EEG information, such as a single channel (2514) or multiple channels of EEG signals. Such sensed EEG information may be obtained via EEG sensor 2012 (FIG. 30A) or derived from information sensed via another sensing modality. In some examples, the EEG information sensed per parameter 2512 comprises sleep state information. In some such examples, the sleep state information may comprise the parameters provided in the later described sleep state portion 2650 of care engine 2500.

In some examples, the sensing portion 2510 may comprise an electrooculogram (EOG) parameter 2524, which relates to receiving, tracking, evaluating, and/or directing sensing of eye movement, eye position, etc., such as via an EOG sensor (e.g. 2024 in FIG. 30A). In some such examples, the sensing element may comprise an optical sensor.

In some examples, this EOG information may be used as part of determining and/or confirming sleep state information, among other CNS information which may be used to sense, diagnose, and/or treat sleep disordered breathing (SDB) behavior. For instance, in some such examples, this EOG information may comprise detection and/or tracking of rapid eye movement (REM) per parameter 2668 (FIG. 32) during sleep, which in turn may be used in differentiating between an awake state, REM state, and/or other sleep states, including various sleep stages.

As further shown in FIG. 32, the care engine 2500 may comprise a sleep state portion 2650 to sense and/or track sleep state information, which may be obtained via the EEG information parameter 2512, in some examples. In some examples, the sleep state portion 2650 may identify and/or track onset (2660) of sleep (e.g. initial sleep onset or sleep onset after WASO) and/or offset (2662) of sleep (e.g. WASO), as well as identify and/or track sleep stages once the patient is asleep. Accordingly, in some examples, the sleep state portion 2650 comprises sleep stage parameter 2666 to identify and/or track various sleep stages (e.g. REM and N1, N2, N3 or S1, S2, S3, S4) of the patient during a treatment portion or during longer periods of time. In some instances, the various stages (e.g. N1-N3 or S1-S4) other than REM sleep may sometimes be referred to as non-REM sleep. The sleep state portion 2650 also may comprise, in some examples, a separate rapid eye movement (REM) parameter 2668 to sense and/or track REM information in association with various aspects of sleep disordered breathing (SDB) care, as further described below and throughout various examples of the present disclosure. In some examples, the REM parameter 2668 may form part of, or be used with, the sleep stage parameter 2666.

In some examples, the sleep state portion 2650 may comprise a wakefulness parameter 2664 to direct sensing of, and/or to receive, track, evaluate, etc. sensing a wakeful state (e.g. WASO) of the patient. An awake state of a patient may be indicative of general non-sleep periods (e.g. daytime) and/or of interrupted sleep events, such as macro-arousals (per parameter 2672 of arousals parameters 2670) associated with a patient waking up to use the restroom (e.g. urinate, etc.), rolling over in bed, waking up in the morning to turn off their alarm, and the like.

Conversely, in some examples, the sleep state portion 2650 may comprise a micro-arousal parameter 2674, by which one may detect and/or track neurological arousals associated with sleep disordered breathing (SDB) events in which a patient experiences a short neurological arousal due to sleep apnea, such as but not limited to obstructive sleep apnea, central sleep apnea, and/or hypopneas. Such SDB-related micro-arousals typically do not result in the patient waking up, in the traditional sense familiar to a lay person. In at least some examples, the stimulation intensity within a treatment period is not varied in response to such SDB-related micro-arousals as one goal of the therapy is for the electrical stimulation to prevent or substantially reduce sleep disordered breathing, which in turn would lessen the frequency and volume of such SDB-related micro-arousals.

In some examples, via at least the sleep state portion 2650 of care engine 2500, the sleep detection method/device may differentiate between wakefulness and sleep disordered breathing (SDB), which occurs during sleep. Among other situations, this differentiation may enable effective neurostimulation therapy such as when a patient is in a sleep position (e.g. laying horizontally or incline position) and the sleep detection arrangement detects a change in sensed data which could possibly be interpreted as a rolling over (e.g. from a supine position onto their side (e.g. lateral decubitus) or vice versa) or as consistent with a SDB behavior. In the case of a bona fide rollover by the patient, such as when getting out of bed, the system will pause the neurostimulation therapy. However, if the detected change may be confirmed as legitimate SDB behavior, then the system/method does not pause the neurostimulation therapy in at least some examples.

With this in mind, in some examples the device/method may differentiate between REM sleep (even where no sleep disordered breathing (SDB) is present) and wakefulness at least because if the patient is in REM sleep, the system avoids pausing neurostimulation therapy for sleep disordered breathing. Conversely, if the patient is in an actual wakeful state, the system should not initiate neurostimulation therapy or may act to pause or to terminate neurostimulation therapy. In some examples, one characteristic feature associated with REM is a lack of body motion, which may sometimes be referred to as paralysis or at least partial paralysis of voluntary muscle control.

In some examples, sleep disordered breathing may occur during REM sleep, such that at least some example device/methods may differentiate sleep disordered breathing from wakefulness and/or differentiate REM sleep from wakefulness. For instance, in some such examples, sensing a lack of body motion may prevent a false positive if/when other parameters (e.g. HR) might otherwise be indicative of wakefulness. For example, during REM sleep stage, sensed information may indicate increased variability in the respiratory period and/or in the heart rate (HR) of the patient.

In some such examples and as previously described, the sleep state information (per sleep state portion 2650) may be used to direct, receive, track, evaluate, diagnose, etc. sleep disordered breathing (SDB) behavior. In some such examples and as previously described, the sleep state information may be used in a closed-loop manner to initiate, terminate, and/or adjust stimulation therapy to treat sleep disordered breathing (SDB) behavior to enhance device efficacy. At least some example closed-loop implementations are further described later in association with at least parameter 2910 in FIG. 32.

For instance, in some examples via sensing wakefulness (2664 in a sleep state portion 2650), stimulation therapy may be terminated automatically. In some examples, via sensing commencement of particular sleep stages (2666), stimulation therapy may be initiated automatically. In some examples, the intensity of stimulation therapy may be adjusted and implemented according to a particular sleep stage and/or particular characteristics within a sleep stage. In some examples, a lower stimulation intensity level may be implemented upon detecting a REM sleep stage. In some examples, stimulation intensity may be decreased in some sleep stages to conserve power and battery life as well as to improve patient comfort and/or therapy utilization.

In some examples, in cooperation with at least sleep stage parameter 2666 of care engine 2500, delivery of a stimulation signal may be toggled among different predetermined intensity levels for each different sleep stage (e.g. N1, N2, N3 or S1, S2, S3, S4, REM).

In addition to the above described sensing parameters, modalities, etc. described in association with FIG. 32, in some examples, the sensing portion 2510 of care engine 2500 comprises an ECG parameter 2520, EMG parameter 2522, accelerometer parameter 2526, pressure parameter 2537, temperature parameter 2538, acoustic parameter 2539 to direct sensing of, and/or to receive, track, evaluate, etc. sensing signals from the previously described ECG sensor 2020, EMG sensor 2022, accelerometer 2026, pressure sensor 2037, temperature sensor 2038, and/or acoustic sensor 2039 in association with FIG. 30A. In some examples, the EMG parameter 2522 may comprise detecting muscle activity and/or motion at intercostal muscles, the upper airway, and/or the tongue, such as described in association with at least FIG. 31A and other examples throughout the present disclosure.

In some examples, the sensing portion 2510 of care engine 2500 (FIG. 32) comprises an impedance parameter 2536 to sense and/or track sensing of impedance within the patient's body to sense motion at (or of) the chest and/or neck and/or other parameters in order to determine sleep-wake status. In addition to or instead of being used to determine sleep-wake status, the impedance parameter 2536 also may be used to sense respiratory information, and/or other information in association with sleep disordered breathing (SDB) care. The impedance parameter 2536 may obtain impedance information from impedance sensor 2036 in FIG. 30A and/or other sensors.

In some examples, sensing portion 2510 of care engine 2500 may comprise a posture parameter 2540 to direct sensing of, and/or to receive, track, evaluate, etc. sensing signals from the previously described posture sensor 2040 in FIG. 30A or other posture, body-position sensor, etc. Like the other parameters of sensing portion 2510, the posture parameter 2540 may be used alone or in combination with other parameters to determine a sleep-wake status of the patient. As previously noted, however, in some example methods (and/or devices) a determination of sleep-wake status may be made without (or independent of) posture information.

In some examples, sensing portion 2510 of care engine 2500 comprises a snoring parameter 2545 to direct sensing of, and/or to receive, track, evaluate, etc. snoring information, which in some examples may be detected and obtained via motion sensing. This sensed snoring information may be used, in some examples, to at least partially determine a sleep-wake status. In one aspect, snoring may be defined as noise associated with each exhalation when respiratory periods are relatively stable and with stable frequency content. Conversely, talking lacks stable respiratory periods and frequency content, and therefore would not be detected as snoring. As noted elsewhere, in some examples the snoring is sensed via acoustic sensor 2039 (FIG. 30A) and/or acoustic parameter 2539 (FIG. 32).

In some examples, sensing portion 2510 of care engine 2500 may comprise a history parameter 2542 by which a history of sensed physiologic information is maintained, and which may be used via comparison parameter 2544 to compare recent sensed physiologic information with older sensed physiologic information. At least some example implementations of using such history parameter 2542 and comparison parameter 2544 are described in association with at least FIGS. 19-20.

In some examples, via care engine 2500, at least some example methods to determine a sleep-wake status may comprise identifying sleep via trends (including variability) in a respiratory rate and/or in a heart rate. In some examples, determination of the sleep-wake status may comprise identifying sleep via a morphology of respiratory cycles, via stability of a respiratory rate, and/or stability in the respiratory morphology. At least some of these examples are further described below in association with at least respiration portion 2580 of care engine 2500.

As shown in FIG. 32, in some examples, care engine 2500 may comprise a respiration portion 2580. In at least some examples, in general terms respiration portion 2580 may direct sensing of, and/or receive, track, and/or evaluate respiratory morphology, including general patterns and/or specific fiducials within a respiratory signal. In some examples, the respiration portion 2580 may operate in cooperation with, or as part of, sensing portion 2510 of care engine 2500 in FIG. 32 and/or sensing portion 2000 (FIG. 30A). At least some aspects of such respiratory morphology managed via respiration portion 2580 may comprise inspiration morphology (parameter 2582) and/or expiration morphology (parameter 2584). In some examples, the respective inspiration morphology parameter 2582 and/or expiration morphology parameter 2584 may comprise amplitude, duration, peak (2586), onset (2588), and/or offset (2590) of the respective inspiratory and/or expiratory phases of the patient's respiratory cycle. In some examples, the detected respiration morphology may comprise transition morphology (2592) such as an inspiration-to-expiration transition and/or an expiration-to-inspiration transition. In some examples, any one or more of these aspects (e.g. peak, onset, offset, magnitude, etc.) of the respective inspiratory and expiratory phases may be used to at least partially determine sleep and/or wakefulness.

For example, the inspiration-to-expiration transition associated with respiration portion 2580 of care engine 2500 may be used as a fiducial to detect and/or track a respiratory rate (and respiratory rate variability), which may be indicative of a change in wake-sleep status. In some examples, changes in a duration of the inspiration-to-expiration transition, changes in peak-to-peak amplitude, and/or changes in the respiratory rate may be indicative of sleep and/or wakefulness, and therefore used to determine a sleep-wake status.

With regard to the sensing, tracking, etc. of respiratory morphologies described above, FIG. 33 is a diagram 3350 schematically representing a respiratory cycle 3360 which illustrates at least some aspects of respiratory morphology, with respiratory cycle 3360 including an inspiratory phase 3362 and an expiratory phase 3370. The inspiratory phase 3362 includes an initial portion 3364 (e.g. onset), inspiratory peak 3365, end portion 3366 (e.g. offset), while expiratory phase 3370 includes an initial portion 3374 (e.g. onset), intermediate portion 3375 (including expiratory peak 3377), and end portion 3376 (e.g. offset). The above-noted peak parameter 2586, onset parameter 2588, and offset parameter 2590 of the inspiration morphology 2582 (in respiration portion 2580 of care engine 2500) corresponds to the inspiration peak 3365, inspiration onset 3364, and inspiration offset 3366 of the respiratory cycle diagram 3350 in FIG. 33, while the above-noted peak parameter 2586, onset parameter 2588, and offset parameter 2590 of the expiration morphology parameter 2584 (in respiration portion 2580 of care engine 2500) corresponds to the expiratory peak 3377, expiratory onset 3374, and expiratory offset 3376 of the respiratory cycle diagram 3350 in FIG. 33.

In the respiratory cycle diagram 3350 in FIG. 33, a first transition 3380 occurs at a junction between the end inspiratory portion 3366 and the initial expiratory portion 3374. In some instances, this transition 3380 may sometimes be referred to as an inspiration-to-expiration transition 3380, which as noted above may be used to determine a sleep-wake status per parameter 2592 of respiration portion 2580 of care engine 2500 in FIG. 32. A second transition 3382 occurs at a junction between the end expiratory portion 3376 and the initial inspiratory portion 3364. In some instances, this transition 3376 may sometimes be referred to as an expiration-to-inspiration transition 3376, which as noted above may be used to determine a sleep-wake status per parameter 2592 of respiration portion 2580 of care engine 2500 in FIG. 32.

In some examples, as shown in FIG. 32 the respiration portion 2580 may comprise a chest wall parameter 2594 to direct sensing of and/or receive, track, evaluate, etc. chest wall behavior of the patient. In some such examples, the chest wall behavior may comprise chest wall motion (e.g. ribcage motion). In some examples, the sensed chest wall motion (e.g. used in determining sleep-wake status) may comprise general motion (e.g. rise and fall) of the chest wall associated with inspiration and expiration of a respiratory cycle as the patient breathes. In some instances, this chest wall motion may comprise intercostal muscle contraction. In some examples, this sensed general chest wall motion (e.g. used in determining sleep-wake status) does not include characteristics such as pectoral muscle contraction and/or signal information (which may be unrelated to breathing and/or cardiac function). Among other uses, the sensed chest motion may be used to determine respiratory information, cardiac information and/or other physiologic information in order to determine a sleep-wake status, as further described throughout various examples of the present disclosure. For instance, one use of the sensed chest motion is to at least partially determine whether respiration is passive or active (e.g. forced), which in turn may be used to determine a sleep-wake status. As just one example aspect of passive respiration, normal exhalation occurs without direct muscular effort, as during normal tidal breathing when air may be expelled from the lungs as a result of the recoil effect of elastic tissues in the chest, lungs, and diaphragm. This behavior would be expected in a sleep state. In contrast, one example of active respiration, which may be associated with an awake state, includes forced exhalation which involves contraction of the abdominal wall, internal intercostal muscles, and diaphragm.

In some examples, as shown in FIG. 32 the respiration portion 2580 may comprise a neck parameter 2595 to direct sensing of and/or receive, track, evaluate, etc. neck movement of the patient, which may be indicative of respiratory information and/or cardiac information regarding the patient, which may be used to determine a sleep-wake status. As previously described, such sensed movement of the neck and/or at the neck may comprise movement such as (but not limited to) motion from the airway and/or blood vessels, impedance, and/or other physiologic phenomenon. For instance, at least some sensed impedance vectors may be measured across the airway, across a vessel, and/or across both.

In some examples, the respiratory portion 2580 may comprise a respiratory rate parameter 2596 to direct sensing of, and/or receive, track, evaluate, etc. respiratory rate information including a respiratory rate, respiratory rate variability 2597, etc., which may be used to determine a sleep-wake status or change in sleep-wake status. In some examples, sensing the respiratory rate (and any associated variability, trends, etc.) may be implemented via sensing and tracking one of the above-noted identifiable parameters (e.g. peak, onset, offset, transition) of respiration morphology per respiratory portion 2580 of care engine 2500.

As shown in FIG. 32, in some examples the care engine 2500 may comprise a cardiac portion 2600. In some examples, in general terms the cardiac portion 2600 may be employed to sense, track, determine, etc. cardiac information, which may be indicative of a sleep-wake status, among other information pertinent to SDB care. In some examples, the cardiac portion 2600 may operate in cooperation with, or as part of, sensing portion 2510 of care engine 2500 (FIG. 32) and/or sensing portion 2000 (FIG. 30A). The cardiac portion 2600 may be employed, alone or in combination with, other elements, modalities, etc. of the care engine 2500. In some examples, the cardiac portion 2600 may employ a single type of sensing or multiple types of sensing in sensing portion 2510, and in some examples, the cardiac portion 2600 may employ other sensing types, modalities, etc. in addition to, or as an alternative to, the particular sensing types, modalities of sensing portion 2510. Moreover, the cardiac portion 2600 may determine, track, etc. a sleep-wake status in cooperation with, or independent of, the respiration portion 2580 of care engine 2500.

In some examples, in general terms the cardiac portion 2600 may direct sensing of, and/or receive, track, evaluate, etc. cardiac signal morphology to at least determine a sleep-wake status. As shown in FIG. 32, in some examples the cardiac portion 2600 comprises an atrial morphology parameter 2610 and/or a ventricular morphology parameter 2612, which may be employed alone, or in combination, to determine a sleep-wake status. In some examples, at least some aspects of the respective atrial and ventricular morphologies (2610, 2612) may comprise detecting contraction (parameter 2620) and/or relaxation (parameter 2622) of the atria and ventricles, respectively. In some such examples, the tracking of the respective contraction and/or relaxation may facilitate determining a sleep-wake status by providing a readily identifiable portion of a cardiac waveform by which heart rate (HR) and/or heart rate variability (HRV) may be detected, tracked, and from which values, trends, etc. of the heart rate or heart rate variability may indicate sleep or wakefulness.

In some examples, at least some aspects of the respective atrial and ventricular morphologies (2610, 2612) may comprise a peak (2630) of an atrial or ventricular contraction, which may be used to determine a sleep-wake status.

In some examples, at least some aspects of the respective atrial and ventricular morphologies (2610, 2612) may comprise an onset (e.g. start) 2632 of an atrial contraction, of an atrial relaxation, of a ventricular contraction, or of a ventricular relaxation. In some examples, at least some aspects of the respective atrial and ventricular morphologies (2610, 2612) may comprise an offset (e.g. termination, end) 2634 of an atrial contraction, an atrial relaxation, a ventricular contraction, or ventricular relaxation.

In some examples, at least some aspects of the respective atrial and ventricular morphologies (2610, 2612) by which a sleep-wake status may be detected may comprise a combination of atrial and ventricular contraction.

In some examples, at least some aspects of the respective atrial and ventricular morphologies (2610, 2612) may comprise a transition (2640), such as a transition between different phases of the cardiac cycle.

In some examples, at least some aspects of the cardiac information (by which a sleep-wake status may be determined) may comprise opening or closing of a heart valve per parameter 2642. In some examples, such detection of opening and/or closing of a heart valve (per parameter 2642) also may be used to help determine the timing and/or occurrence of an onset and/or offset of a contraction (or relaxation) of an atria or ventricles in association with parameters 2610, 2612, 2620, 2622, 2630, 2632, 2634.

In some examples, cardiac information may comprise heart motion 2644, from which the above-described cardiac morphology parameters may be determined. The heart motion 2644 may be obtained via one or more of the various sensing modalities (e.g. accelerometer, EMG, etc.) described in association with at least FIG. 30A.

As further shown in FIG. 32, in some examples, cardiac information may comprise heart rate parameter 2645 to direct sensing of, and/or receive, track, evaluate, etc. heart rate information including a heart rate (HR), heart rate variability (HRV) 2646, etc., which may be used to determine a sleep-wake status or change in sleep-wake status. In some examples, sensing the heart rate (and any associated variability, trends, etc.) may be implemented via sensing and tracking one of the above-noted identifiable parameters (e.g. peak, onset, offset, transition) of cardiac morphology per cardiac portion 2600.

In some examples, at least some of the above-described cardiac information may be determined, at least partially, according to heart sounds (e.g. S1, S2, etc.), which may be sensed acoustically (e.g. 2039 in FIG. 30A; 2539 in FIG. 32).

In some examples, sleep-wake status may be determined via a combination of sensed respiratory features and sensed cardiac features. At least some aspects of use of this combination of information are previously described in association with at least FIGS. 13-20, and elsewhere throughout examples of the present disclosure.

As further shown in FIG. 32, in some examples the care engine 2500 comprises a SDB parameters portion 2800 to direct sensing of, and/or receive, track, evaluate, etc. parameters particularly associated with sleep disordered breathing (SDB) care. For instance, in some examples, the SDB parameters portion 2800 may comprise a sleep quality portion 2810 to sense and/or track sleep quality of the patient in particular relation to the sleep disordered breathing behavior of the patient. Accordingly, in some examples the sleep quality portion 2810 comprises an arousals parameter 2812 to sense and/or track arousals caused by sleep disordered breathing (SDB) events with the number, frequency, duration, etc. of such arousals being indicative of sleep quality (or lack thereof). In some such examples, such arousals may correspond to micro-arousals as described in association with at least parameter 2674 in sleep state portion 2650 of care engine 2500 in FIG. 32.

In some examples, the sleep quality portion 2810 comprises a state parameter 2814 to sense and/or track the occurrence of various sleep states (including sleep stages) of a patient during a treatment period or over a longer period of time. In some such examples, the state parameter 2814 may cooperate with, form part of, and/or comprise at least some of substantially the same features and attributes as sleep state portion 2650 of care engine 2500.

In some examples, the SDB parameters portion 2800 comprises an AHI parameter 2830 to sense and/or track apnea-hypopnea index (AHI) information, which may be indicative of the patient's sleep quality. In some examples, AHI information is sensed throughout each of the different sleep stages experienced by a patient, with such sensed AHI information being at least partially indicative of a degree of sleep disordered breathing (SDB) behavior. In some examples, the AHI information is obtained via a sensing element, such as one or more of the various sensing types, modalities, etc. in association with at least sensing portion 2000 (FIG. 30A) and/or sensing portion 2510 (FIG. 32), which may be implemented as described in various examples of the present disclosure. In some examples, AHI information may be sensed via a sensing element, such as an accelerometer located in either the torso or chin/neck region with the sensing element locatable and implemented as described in various examples of the present disclosure. In some examples, a combination of accelerometer-based sensing and other types of sensing may be employed to sense and/or track AHI information. In some examples, the AHI information is obtained via sensing modalities (e.g. ECG, impedance, EMG, etc.) other than via an accelerometer.

In some examples, determination of sleep-wake status may be implemented via a probability portion 3200 of care engine 2500 in FIG. 32. In some such examples, via a selection parameter 3210, the probability portion 3200 may enable selective inclusion or selective exclusion of at least some sleep-wake determination parameters without directly affecting the general operation of determining sleep-wake status. In some examples, via the probability function 3200, a sensitivity parameter 3220 may be adjusted by a patient, clinician, caregiver to increase or decrease a sensitivity of determining the sleep-wake status via a particular parameter. In some examples, a data model parameter 3230 (e.g. machine learning) may be implemented to assess and modify adjustments to probabilistic determinations of the sleep-wake status, including but not limited to, adjustments to any amplitude thresholds, duration thresholds, etc. associated with a probabilistic determination of sleep-wake status. In some examples, employing such probabilistic determinations may permit more granular controls of a patient's individual signals (used in combination to make the determination of sleep-wake status), which in turn, may enable balancing simple control with the capability of complex control and sensor flexibility when desired.

In some examples, via at least the data model parameter 3230, the care engine 2500 may comprise and/or access a neural network resource (e.g. deep learning, convolutional neural networks, etc.) to identify patterns indicative of sleep from a single sensor or multiple sensors. As further described later in association with at least FIGS. 40-42, a data model may be constructed and/or trained via other example methods. In some examples, a decision tree-based expert resource also could be used to combine sensors or neural network output with other signals such as time of day or remote inputs/usage. One example implementation of using a data model (e.g. machine learning, other), such as via parameter 3230, is further described later in association with at least FIGS. 40-42. At least some other example implementations are described throughout the present disclosure.

In some examples, via probability portion 3200, care engine 2500 may assign and apply a weight (parameter 3240) to be associated with each signal in order to increase (or decrease) the relative importance of a particular sensor signal in determining sleep-wake status.

In some examples, via a temporal emphasis parameter 3250 different thresholds may be selected for different times of a 24 hour daily period. For instance, during a first period (e.g. daytime such as Noon) some parameters may be de-emphasized and/or other parameters emphasized, while during a second period (e.g. late evening such as 10 pm), some parameters may be emphasized in determining sleep-wake status while other parameters are de-emphasized. Alternatively, during the first period, the sensitivity of most or all parameters (for determining sleep-wake status) may be decreased and during the second period, the sensitivity of some or all parameters (for determining sleep-wake status) may be increased.

In some such examples, this adjustability via the temporal emphasis parameter 3250 may enhance sleep-wake determinations for a patient having non-standard sleep periods, such as a graveyard shift worker (e.g. works 11 pm-7 am), because their intended sleep period (e.g. 8 am-3 pm) conflicts with a conventional sleep period (e.g. 10 pm-6 am).

In some examples, the probability function 3200 of care engine 2500 may implement a probabilistic determination of sleep-wake status based on sensing motion at (or of) the chest, neck, and/or head. In some such examples, an accelerometer and/or other sensors (e.g. impedance, EMG, etc.) may be employed to sense motion at (or of) the chest, neck, and/or head. In some such examples, per a differentiation parameter 3260, where sensing is performed via a sensor (e.g. accelerometer) with multiple signal components (e.g. a multiple axis accelerometer) or captures a signal (e.g. ECG) from which multiple different signals may be derived, an example method may comprise dividing a signal associated with sensing the physiologic information into a plurality of different signals with each respective signal representing a different sleep-wake determination parameter. Stated differently, multiple components within a signal are differentiated into distinct and separate signals, each of which may be indicative of sleep-wake status. A probability of sleep-wake status is then determined based on assessing the respective different signals associated with the respective different sleep-wake determination parameters. As noted above, in some examples each respective different signal may comprise one axis of a multiple axis accelerometer (e.g. in which each axis is orthogonal to other axes) or may comprise a single axis accelerometer (when multiple single-axis accelerometers are employed). In some such examples, a different processing method or technique may be applied to at least some of the signal components (e.g. sleep determination parameters).

As shown in FIG. 32, in some examples the care engine 2500 may comprise an activation portion 3000, which in general terms may control activation of a medical device, such as a pulse generator, whether implantable (e.g. IPG) or external or some combination thereof. In some such examples, nerve stimulation delivery via the medical device may be activated and terminated automatically (3010), with such activation and termination based on sleep-wake status. In some such examples, the sleep-wake status is determined automatically via care engine 2500.

Accordingly, in some examples, via automatic parameter 3010, at least some example methods and/or devices for determining sleep-wake status may be used to automatically initiate a treatment period (e.g. upon automatically detecting sleep) and to automatically terminate a treatment period (e.g. upon automatically detecting wakefulness).

In some examples in which the automatic determination of a sleep-wake status is unavailable or deactivated by the patient (or clinician or caregiver), then per remote parameter 3012 the treatment period may comprise a period of time beginning with the patient using a remote control to turn on the therapy device and ending with the patient turning off the device via the remote control. In some examples, per remote parameter 3012, a treatment period may be initiated and/or terminated based on at least one of a degree of ambient light sensed via a remote control, a degree or type of motion sensed by the remote control, and/or the above-described therapy activation (e.g. on, off) implemented via the remote control. In some examples, the remote control may comprise the remote control 4340 shown in FIG. 36. It will be understood that, in some examples, detecting the degree of ambient light and/or the degree or type of motion of the remote control may be used as part of other features described herein to perform an automatic determination of sleep-wake status, which in turn may determine automatic initiation, termination, pause, adjustment, etc. of a treatment period in which neurostimulation therapy is applied. In some examples, remote control parameter 3012 may be implemented in association with method 788 in FIG. 26I.

In some examples in which the automatic determination of a sleep-wake status is unavailable or deactivated by the patient (or clinician or caregiver), then per app parameter 3013 the treatment period may comprise a period of time beginning with the patient using an app to turn on the therapy device and ending with the patient turning off the device via the app. In some examples, per app parameter 3013, a treatment period may be initiated and/or terminated based on at least one a degree of ambient light sensed via app, a degree or type of motion sensed by the mobile device, and/or the above-described therapy activation (e.g. on, off) implemented via the app on the mobile device. In some examples, the app may comprise the app 4330 shown in FIG. 36, which may be implemented via a mobile device 4320 (FIG. 36), such as a mobile smart phone, tablet, phablet, smart watch, etc. The mobile device may comprise a control portion, user interface (e.g. display) to operate the app, and the mobile device may comprise sensor(s) to sense the above-described features (e.g. motion, ambient light, sounds, etc.) in a manner to enable the app to perform sleep-wake determination at least partially based on the use (or non-use) of the mobile device.

In some examples, the sensor(s) of a remote control and/or mobile device may comprise an accelerometer, gyroscope, and/or other motion detector.

However, in some examples where automatic determination of sleep-wake status is unavailable (or deactivated), via the temporal parameter 3014 the treatment period may begin automatically at a selectable, predetermined start time (e.g. 10 pm) and may terminate at a selectable, predetermined stop time (e.g. 6 am)

In one aspect, the treatment period corresponds to a period during which a patient is sleeping such that the stimulation of the upper airway patency-related nerve and/or central sleep apnea-related nerve is generally not perceived by the patient and so that the stimulation coincides with the patient behavior (e.g. sleeping) during which the sleep disordered breathing behavior (e.g. central or obstructive sleep apnea) would be expected to occur. Accordingly, to avoid enabling stimulation prior to the patient falling asleep, in some examples stimulation can be enabled during the treatment period after expiration of a timer started upon the automatic sleep detection. To avoid continuing stimulation after the patient wakes, stimulation can be disabled upon the automatic detection of wakefulness. Accordingly, in at least some examples, these periods may be considered to be outside of the treatment period or may be considered as a startup portion and wind down portion, respectively, of a treatment period.

In some examples, via a boundary parameter 3016 a selectable, predetermined first time marker (e.g. 10 pm) may be used as a limit or boundary to prevent automatic initiation of a treatment period (based on automatic detection of sleep) before the first time marker, and a selectable, predetermined second time marker (e.g. 6 am) may be used as a limit or boundary to ensure automatic termination of a treatment period to prevent continuance of a treatment period after the second time marker. Via such example arrangements, the treatment period may be initiated automatically via automatic sleep detection and/or may be terminated automatically via automatic wakefulness detection, while providing assurance to the patient of a treatment period not being initiated during normally wakeful periods, or not extending beyond their normal sleep period.

In some examples, determining sleep-wake status in association with boundary parameter 3016 may comprise and/or be combined with at least the features and attributes as previously described in association with method 780 in FIG. 26A, as well as in association with temperature parameter 2038 (FIG. 30A) as previously described.

However, in some instances, via a physical parameter 3018 a user may take physical steps to cause activation (or deactivation) of a treatment period for the implantable medical device. For instance, via the activation portion 3000 and physical parameter 3018, the care engine 2500 may receive physical input such as tapping of the chest (or neck or head) or tapping over the implant to activate or deactivate the device. Alternatively, a user may use a patient remote control function 3012 to activate or deactivate the implantable medical device, which in turn may activate or deactivate delivery of nerve stimulation. In some such examples, activation or deactivation of the treatment period (in which nerve stimulation is applied) may be implemented via physical motion of a remote control or a mobile device (e.g. hosting an app). In some instances, via a clinician programmer or remote control, this physical feature (3018) may be activated or deactivated at the discretion of the clinician or user.

As further shown in FIG. 32, in some examples care engine 2500 comprises a stimulation portion 2900 to control stimulation of target tissues, such as but not limited to an upper airway patency nerve, to treat sleep disordered breathing (SDB) behavior. In some examples, the stimulation portion 2900 comprises a closed loop parameter 2910 to deliver stimulation therapy in a closed loop manner such that the delivered stimulation is in response to and/or based on sensed patient physiologic information.

In some examples, the closed loop parameter 2910 may be implemented as using the sensed information to control the particular timing of the stimulation according to respiratory information, in which the stimulation pulses are triggered by or synchronized with specific portions (e.g. inspiratory phase) of the patient's respiratory cycle(s). In some such examples and as previously described, this respiratory information may be determined via a single type of sensing or multiple types of sensing via sensing portion 2000 (FIG. 30A) and sensing portion 2510 (FIG. 32).

In some examples in which the sensed physiologic information enables determining (at least) a sleep-wake status, the closed loop parameter 2910 may be implemented to initiate, maintain, pause, adjust, and/or terminate stimulation therapy based on the determined sleep-wake status (including particular sleep stages).

As further shown in FIG. 32, in some examples the stimulation portion 2900 comprises an open loop parameter 2925 by which stimulation therapy is applied without a feedback loop of sensed physiologic information. In some such examples, in an open loop mode the stimulation therapy is applied during a treatment period without (e.g. independent of) information sensed regarding the patient's sleep quality, sleep state, respiratory phase, AHI, etc. In some such examples, in an open loop mode the stimulation therapy is applied during a treatment period without (i.e. independent of) particular knowledge of the patient's respiratory cycle information.

However, in some such examples, some sensory feedback may be utilized to determine, in general, whether the patient should receive stimulation based on a severity of sleep apnea behavior.

As further shown in FIG. 32, in some examples the stimulation portion 2900 comprises an auto-titration parameter 2920 by which an intensity of stimulation therapy can be automatically titrated (i.e. adjusted) to be more intense (e.g. higher amplitude, greater frequency, and/or greater pulse width) or to be less intense (e.g. lower amplitude, lower frequency, and/or lower pulse width) within a treatment period.

In some such examples and as previously described, such auto-titration may be implemented based on sleep quality and/or sleep state information, which may be obtained via sensed physiologic information, in some examples. It will be understood that such examples may be employed with synchronizing stimulation to sensed respiratory information (i.e. closed loop stimulation) or may be employed without synchronizing stimulation to sensed respiratory information (i.e. open loop stimulation).

In some examples, at least some aspects of the auto-titration parameter 2920 may comprise, and/or may be implemented, via at least some of substantially the same features and attributes as described in Christopherson et al., SYSTEM FOR TREATING SLEEP DISORDERED BREATHING, issued as U.S. Pat. No. 8,938,299 on Jan. 20, 2015, and which is hereby incorporated by reference in its entirety.

With regard to the various examples of the present disclosure, in some examples, delivering stimulation to an upper airway patency nerve is to cause contraction of upper airway patency-related muscles. In some such examples, the contraction comprises a suprathreshold stimulation, which is in contrast to a subthreshold stimulation (e.g. mere tone) of such muscles. In one aspect, a suprathreshold intensity level corresponds to a stimulation energy greater than the nerve excitation threshold, such that the suprathreshold stimulation may provide for maximum upper-airway clearance (i.e. patency) and obstructive sleep apnea therapy efficacy.

In some examples, at least some example methods may comprise identifying, maintaining, and/or optimizing a target stimulation intensity (e.g. therapy level) without intentionally identifying a stimulation discomfort threshold at the time of implantation or at a later point in time after implantation.

In some examples, upon determining sleep according to a minimum predetermined confidence level, an amplitude (e.g. intensity) of the stimulation signal may start at a lower value and then be increased to higher values in a ramped manner. In some such examples, the increases in amplitude (up to a desired/target value) may be made dependent on additional or further predetermined confidence levels. However, if it is later determined that sleep is not occurring but rather that the patient is in a quiet, restful awake state, then the stimulation may be terminated or ramped down while still in the ramping phase, prior to reaching a target stimulation amplitude. Among other applications, this example method may be beneficial for patients with cardiac or respiratory disorders at least because the cardiac morphologies and/or respiratory morphologies (from which sleep may be detected) may be more complex such that accurate detection of actual sleep may be more challenging in such patients.

As noted above in association with the boundary parameter 3016 of the activation portion 3000, a clock or time keeping element within (or in communication with) a medical device (e.g. which may be implantable in some examples such as (but not limited to) IPG 2133) may be used to implement boundaries or limit for when stimulation therapy (within a treatment period) may be automatically initiated or terminated via automatic sleep detection (or wake detection) per determining a sleep-wake status. In some examples, the time-based boundaries may be based on patient behaviors and/or direct clinician programming. In some examples, such tracked patient behavior may be used as input to a probabilistic model of determining a sleep-wake status. In some examples, the time-based boundaries also may be based, at least in part, on a history of patient activities.

In some examples, the time-based boundaries may account for daylight savings time and travel (e.g. different time zones), and may be adjusted via a patient remote control or physical tapping on the chest. In some such examples, a time-based boundary parameter may comprise one of multiple inputs used to determine sleep-wake status, and which may increase reliability in determining sleep-wake status in a variety of environments (rather than a single time-place environment such as only a patient bedroom).

In some examples, boundary parameter 3016 of activation portion 3000 in FIG. 32 may comprise criteria which are not strictly time-based (e.g. time of day). For instance, in some examples the boundary parameter 3016 may be implemented based on a number, type, and/or duration of various sleep stages associated with a single treatment period (e.g. a night's sleep). For instance, an example method may determine a boundary or an end limit of a treatment period according to observing a certain number (e.g. 4 or 5) of REM sleep periods, stage 4 sleep periods, or stage 3 sleep periods, etc. In some such examples, the number of particular sleep stage periods may be selectable. In some examples, the boundary may be based on a selectable percentage that a patient spends in one or more particular sleep stages.

In some examples, upon detecting a sleep state (per a sleep-wake status) a neurostimulation signal may be applied to a phrenic nerve, in order to treat central sleep apnea. In some examples, determining a sleep-wake status may be used to control initiation and/or termination of stimulation of both an upper airway patency nerve (e.g. hypoglossal nerve) and a diaphragm control nerve (in a manner coordinated relative to each other) to treat sleep disordered breathing.

In some examples, the stimulation portion 2900 may operate cooperatively with at least the respiration portion 2580 and/or the sensing portion 2510 of care engine 2500 (e.g. such as in association with sensing portion 2000 in FIG. 30A) to determine efficacy of stimulation, and/or whether a flow limitation exists, by evaluating a flow response within a single respiratory cycle. Such evaluation stands in contrast to performing such evaluation on a cycle-to-cycle basis, such as looking at the respiratory signal from a peak of an inspiratory phase of one cycle to a peak of an inspiratory phase of another cycle.

For instance, in the example method, if the stimulation portion 2900 were to cause a change in the stimulation intensity level (e.g. increase or decrease) during the inspiratory phase, one feature of the care engine 2500 may comprise determining whether a substantial change (e.g. 10%, 15%, 20%, or more) in the flow response occurred.

In some such examples of stimulation portion 2900 in evaluating whether stimulation therapy is efficacious (based on a flow response of the inspiratory phase within a single respiratory cycle versus from cycle-to-cycle), some example methods may comprise determining whether a change (e.g. a substantial change) in the flow response were to occur upon a complete termination of stimulation or upon initiation of stimulation (such as when no stimulation was previously occurring) during an inspiratory phase of a single respiratory cycle.

In some examples, care engine 2500 in FIG. 32 may comprise an initial use function 3100, which in some examples may automatically enhance determination of sleep-wake status. In some such examples, via initial use function 3100 a method and/or device for SDB care may omit a manual training period and instead automatically “normalize” use of the method and/or device for a particular patient. For instance, in some examples, determination of a sleep-wake status may begin with default parameters or may begin with parameters collected at the time of implant of a SDB care device in the patient. In some examples, determination of a sleep-wake status may be performed initially with no default parameters. In some such examples, when wakefulness is detected, sensing portion 2000 (FIG. 30A) and/or care engine 2500 (FIG. 32) may collect respiratory information, motion information, and/or posture information, etc. associated with wakefulness, which in turn may allow for more sensitive detection of sleep in determining a sleep-wake status. In some examples, detection of wakefulness may comprise detecting gross body motion, such as but not limited to walking, swallowing, torso motion, etc. In some examples, a gravity vector is established at the time of implanting the SDB care device.

With this in mind, per the initial use function 3100, such automatic normalization may comprise omitting the use of absolute thresholds and instead perform determination of sleep-wake status (e.g. detection of onset of sleep) on the basis of percentage change in sensed values. Moreover, in some examples, sensing of various physiologic phenomenon (e.g. respiration, cardiac, etc.) may be used to determine a highest value or lowest value of such physiologic phenomenon and then use such end-of-the-range values to adjust thresholds accordingly.

It will be understood that the various parameters, functions, portions, etc. shown and described in association with FIG. 32 are not limited to the particular groupings, relationships, etc. shown in FIG. 32, but may be arranged in groupings, relationships, etc. other than shown in FIG. 32. Moreover, it will be understood that the care engine 2500 (or portions thereof) in FIG. 32 may be implemented with just some (i.e. not all) of the portions, elements, parameters, etc. shown in FIG. 32.

With reference to at least care engine 2500 in FIG. 32 and the example methods and/or devices described throughout the present disclosure, it will be understood that such engines, methods, and/or devices (and components, portions, etc. thereof) for determining a sleep-wake status also may be used for quantifying activity levels and assessing related health parameters.

FIG. 34A is a block diagram schematically representing an example control portion 4000. In some examples, control portion 4000 provides one example implementation of a control portion forming a part of, implementing, and/or generally managing stimulation elements, power/control elements (e.g. pulse generators, microstimulators), sensors, and related elements, devices, user interfaces, instructions, information, engines, elements, functions, actions, and/or methods, as described throughout examples of the present disclosure in association with FIGS. 1A-33.

In some examples, control portion 4000 includes a controller 4002 and a memory 4010. In general terms, controller 4002 of control portion 4000 comprises at least one processor 4004 and associated memories. The controller 4002 is electrically coupled to, and in communication with, memory 4010 to generate control signals to direct operation of at least some of the stimulation elements, power/control elements (e.g. pulse generators, microstimulators) sensors, and related elements, devices, user interfaces, instructions, information, engines, elements, functions, actions, and/or methods, as described throughout examples of the present disclosure. In some examples, these generated control signals include, but are not limited to, employing instructions 4011 and/or information 4012 stored in memory 4010 for at least determining sleep-wake status of a patient, including particular sleep stages in some examples. In some examples, the sleep-wake determination may include determining initial sleep onset, wake after sleep onset (WASO), and/or sleep onset after wake (e.g. WASO). Such sleep-wake determination may comprise part of directing and managing treatment of sleep disordered breathing such as obstructive sleep apnea, hypopnea, and/or central sleep apnea, with such sleep-wake determination also comprising sensing physiologic information including but not limited to electrical brain activity, respiratory information, cardiac information, and/or monitoring sleep disordered breathing, etc. as described throughout the examples of the present disclosure in association with FIGS. 1A-33 and 34B-42. In some instances, the controller 4002 or control portion 4000 may sometimes be referred to as being programmed to perform the above-identified actions, functions, etc. such that the controller 4002, control portion 4000 and any associated processors may sometimes be referred to as being a special purpose computer, control portion, controller, or processor. In some examples, at least some of the stored instructions 4011 are implemented as, or may be referred to as, a care engine, a sensing engine, monitoring engine, and/or treatment engine. In some examples, at least some of the stored instructions 4011 and/or information 4012 may form at least part of, and/or, may be referred to as a care engine, sensing engine, monitoring engine, and/or treatment engine.

In response to or based upon commands received via a user interface (e.g. user interface 4040 in FIG. 35) and/or via machine readable instructions, controller 4002 generates control signals as described above in accordance with at least some of the examples of the present disclosure. In some examples, controller 4002 is embodied in a general purpose computing device while in some examples, controller 4002 is incorporated into or associated with at least some of the stimulation elements, power/control elements (e.g. pulse generators, microstimulators), sensors, and related elements, devices, user interfaces, instructions, information, engines, functions, actions, and/or methods, etc. as described throughout examples of the present disclosure.

For purposes of this application, in reference to the controller 4002, the term “processor” shall mean a presently developed or future developed processor (or processing resources) that executes machine readable instructions contained in a memory. In some examples, execution of the machine readable instructions, such as those provided via memory 4010 of control portion 4000 cause the processor to perform the above-identified actions, such as operating controller 4002 to implement the sensing, monitoring, determining, treatment, etc. as generally described in (or consistent with) at least some examples of the present disclosure. The machine readable instructions may be loaded in a random access memory (RAM) for execution by the processor from their stored location in a read only memory (ROM), a mass storage device, or some other persistent storage (e.g. non-transitory tangible medium or non-volatile tangible medium), as represented by memory 4010. In some examples, the machine readable instructions may comprise a sequence of instructions, a processor-executable data model (e.g. machine learning, other), or the like. In some examples, memory 4010 comprises a computer readable tangible medium providing non-volatile storage of the machine readable instructions executable by a process of controller 4002. In some examples, the computer readable tangible medium may sometimes be referred to as, and/or comprise at least a portion of, a computer program product. In some examples, hard wired circuitry may be used in place of or in combination with machine readable instructions to implement the functions described. For example, controller 4002 may be embodied as part of at least one application-specific integrated circuit (ASIC), at least one field-93 programmable gate array (FPGA), and/or the like. In at least some examples, the controller 4002 is not limited to any specific combination of hardware circuitry and machine readable instructions, nor limited to any particular source for the machine readable instructions executed by the controller 4002.

In some examples, control portion 4000 may be entirely implemented within or by a stand-alone device.

In some examples, the control portion 4000 may be partially implemented in one of the sensing devices, monitoring devices, stimulation devices, apnea treatment devices (or portions thereof), etc. and partially implemented in a computing resource separate from, and independent of, the apnea treatment devices (or portions thereof) but in communication with the apnea treatment devices (or portions thereof). For instance, in some examples control portion 4000 may be implemented via a server accessible via the cloud and/or other network pathways. In some examples, the control portion 4000 may be distributed or apportioned among multiple devices or resources such as among a server, an apnea treatment device (or portion thereof), and/or a user interface.

In some examples, control portion 4000 includes, and/or is in communication with, a user interface 4040 as shown in FIG. 35.

FIG. 34B is a diagram schematically illustrating at least some example implementations of a control portion 4020 by which the control portion 4000 (FIG. 34A) can be implemented, according to one example of the present disclosure. In some examples, control portion 4020 is entirely implemented within or by an IPG assembly 4025, which has at least some of substantially the same features and attributes as a pulse generator (e.g. power/control element, microstimulator) as previously described throughout the present disclosure. In some examples, control portion 4020 is entirely implemented within or by a remote control 4030 (e.g. a programmer) external to the patient's body, such as a patient control 4032 and/or a physician control 4034. In some examples, the control portion 4000 is partially implemented in the IPG assembly 4025 and partially implemented in the remote control 4030 (at least one of patient control 4032 and physician control 4034).

FIG. 35 is a block diagram schematically representing user interface 4040, according to one example of the present disclosure. In some examples, user interface 4040 forms part of and/or is accessible via a device external to the patient and by which the therapy system may be at least partially controlled and/or monitored. The external device which hosts user interface 4040 may be a patient remote (e.g. 4032 in FIG. 34B), a physician remote (e.g. 4034 in FIG. 34B) and/or a clinician portal. In some examples, user interface 4040 comprises a user interface or other display that provides for the simultaneous display, activation, and/or operation of at least some of the stimulation elements, power/control elements (e.g. pulse generators, microstimulators), sensors, and related elements, devices, user interfaces, instructions, information, engines, functions, actions, and/or method, etc., as described in association with FIGS. 1A-42. In some examples, at least some portions or aspects of the user interface 4040 are provided via a graphical user interface (GUI), and may comprise a display 4044 and input 4042.

FIG. 36 is a block diagram 4300 which schematically represents some example implementations by which a medical device (MD) 4310, such as a pulse generator and/or sensing monitor (either or both of which may be implantable in some examples), may communicate wirelessly with external devices outside the patient. As shown in FIG. 36, in some examples, the IMD 4310 may communicate with at least one of patient app 4330 on a mobile device 4320, a patient remote control 4340, a clinician programmer 4350, and a patient management tool 4360. The patient management tool 4360 may be implemented via a cloud-based portal 4362, the patient app 4330, and/or the patient remote control 4340. Among other types of data, these communication arrangements enable the IMD 4310 to communicate, display, manage, etc. sleep/wake data for patient management as well as to allow for adjustment to the detection method if/where needed.

It will be understood that at least some of the various devices/elements 4320, 4340, 4350, patient management tool 4360 also may communicate with each other, with or without communicating with the medical device 4310.

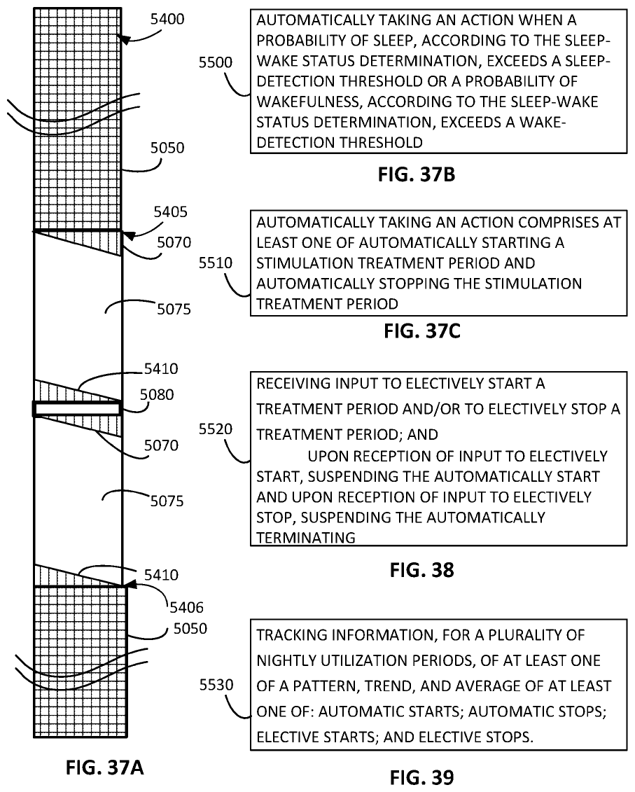

As shown in FIG. 37A, in some examples the user interface 4040 of FIG. 35 also may display and/or report the use of ramped initiation, ramped transitions in and out of a pause in therapy, and/or a ramped termination of stimulation for a given treatment period. For example, as shown in the schematic representation in FIG. 37A, a daily display portion 5400 may comprise various graphic identifiers, such as a wakefulness period 5050, automatic start (e.g. auto-start) instances 5070, on period 5075, etc.

In some example methods, at least some of the start, stop, pauses of stimulation within a treatment period may be implemented in a ramped manner with the display portion 5400 in FIG. 37A schematically representing these implementations. For instance, an automatic start of stimulation may comprise a ramped increase (from zero) to a target stimulation intensity as represented via the triangular shaped ramp symbol shown at 5070. This representation immediately indicates to a viewer the ramped manner in which the stimulation intensity was implemented. The ramped increase may occur at the beginning of a treatment period (e.g. 5405). Similarly, the triangular-shaped ramp symbol 5410 represents a ramped decrease of stimulation intensity from the target level (or another non-zero level) to zero, such as when stimulation is terminated (e.g. at 5406) or when stimulation is to be paused (e.g. at 5080). It will be understood that the representation of a ramped increase or decrease of stimulation intensity may be implemented via shapes other than a triangle.

The gradual ramped initiation or termination of stimulation therapy may enhance a patient's comfort by avoiding abrupt initiation, pause, or cessation of stimulation therapy. Among other features, the ramped implementation may increase the likelihood of patient compliance and appreciation for SDB care.

In some examples, at least some of the features and attributes associated with at least the methods and/or devices represented via FIG. 37A may be implemented via at least some features and attributes of the example methods described hereafter in association with FIGS. 37B-39. In some examples, the methods described in association with FIGS. 37B-39 may be implemented via devices and elements other than those shown in at least FIG. 37A.

It will be understood that FIG. 37A schematically represent at least some aspects of patient's experience of, operation of a device, and/or a method of treating a patient for sleep apnea. Accordingly, at least some aspects of at least FIG. 37A schematically represent via a method such as example method, as shown at 5500 in FIG. 37B, which comprises automatically taking an action when a probability of sleep, according to the sleep-wake status determination, exceeds a sleep-detection threshold or a probability of wakefulness, according to the sleep-wake status determination, exceeds a wake-detection threshold. In some examples, as shown at 5510 in FIG. 37C, automatically taking an action comprises at least one of automatically starting a stimulation treatment period and automatically stopping the stimulation treatment period. In some such examples, the term “non-sleep” may correspond to a probability of sleep remaining below a sleep detection threshold, while in some such examples, the term “non-wake” may correspond to a probability of wakefulness remaining below a wake detection threshold.

In some such examples (at 5510 in FIG. 37C), an example method may further comprise, as shown at 5520 in FIG. 38, receiving input to electively start a treatment period and/or to electively stop a treatment period; and upon reception of input to electively start, suspending the automatically start and upon reception of input to electively stop, suspending the automatically terminating.

In some examples, as shown at 5530 in FIG. 39, a method (associated with the actions/methods in FIGS. 37B-38) may further comprise tracking information, for a plurality of nightly utilization periods, of at least one of a pattern, trend, and average of at least one of: automatic starts; automatic stops; elective starts; and elective stops. It will be understood that other (or additional) nightly utilization parameters described in association with FIG. 37A may be tracked per method 5530 in FIG. 39.

FIG. 40 is a block diagram schematically representing an example arrangement 7400 to implement a data model, such as (but not limited to) a machine learning model for supporting and/or implementing determination of a sleep-wake status (e.g. initial sleep onset, WASO, and/or sleep onset after WASO). In some examples, the data model may comprise a machine learning element, which may comprise a convolutional neural network, deep neural network, deep neural learning, and the like. It will be understood that in some examples the machine learning element may be implemented via other forms of artificial intelligence tools. The data model element may be implemented as part of, or in a complementary manner with, data model parameter 3230 in FIG. 32.

In some examples, the data model may comprise a heuristic data model or other data model that may be manually tuned. For example, the inputs and outputs of the heuristic data model or other data model may be manually selected and/or the weights applied to each input and/or output may be manually adjusted. In some examples, the heuristic data model or other data model may be manually tuned by a physician, a patient, and/or other person based on observations (e.g. sleep study), feedback (e.g. survey), etc. of and/or from a patient.

In some examples, such a data model arrangement may be used in analyzing sensed physiologic phenomenon (e.g. respiratory signals, cardiac signals, etc.) to determine a pattern(s) indicative of a sleep state (e.g. onset, onset latency, onset latency variability, offset, various sleep stages) and/or pattern(s) indicative of wakefulness (e.g. onset, offset). In some examples, at least part of this analysis may comprise comparing stored signal patterns with current or recent signal patterns.

The output of the data model arrangement 7400 may be provided to, or as, a comprehensive sleep-wake status determination at 7643. It will be understood that in some examples, the output of the data model arrangement 7400 may be the sole basis on which a comprehensive sleep-wake status determination is implemented. However, in some examples, the output of the data model arrangement 7400 may comprise just one input in a comprehensive sleep-wake status determination.

In some examples, the data model arrangement 7400 as represented in FIG. 40 may comprise a trained (or constructed) data model (e.g. trained deep learning model), which may be trained (or constructed) prior to its operation. As further shown in the example arrangement (e.g. example method or device) in FIG. 40, in some examples the training may be performed at least partially via a resource 7410. In some examples, resource 7410 may be external to patient's body and/or external to a medical device 7420 (whether implantable and/or external). The medical device 7420 may comprise a sensor (e.g. a sensor of sensing portion 2000 of FIG. 30A) and control portion 4000 (FIG. 34A), among other components, features, etc. In some examples, after such training, the trained (or constructed) data model may be imported into the medical device 7420 for use in determining a sleep-wake status and/or sleep onset latency information (including but not limited to initial sleep onset, WASO, and sleep onset after WASO).

In some examples, the resource(s) 7410 (FIG. 40) may comprise a computing resource 7414 sized and scaled to perform various forms of training/constructing and/or maintaining the data model. In some examples, the resource(s) 7410 may comprise a data store 7412, such as (but not limited to) a large data set of stored sleep information for many patients, which may comprise acceleration signal component information, etc. relating to different non-physiologic parameters and physiologic parameters, such as but not limited to cardiac information, respiratory information, motion/activity information, posture information, etc. It will be understood that any one or more of the sensor modalities disclosed within and throughout the present disclosure also may contribute to the data store 7412. In some examples, the stored sleep-related data may be specific to the patient in which the trained data model may be imported, such as being imported into or as element within a medical device (e.g. 7420).

With this in mind, in some examples the data model element may be trained (i.e. constructed) via the resource 7410 according to the example arrangement (e.g. method and/or device) 7500 in FIG. 41. As shown in FIG. 41, known inputs 7510 sensed via an accelerometer (e.g. implantable in some examples) and/or other sensing modalities and a known output 7540 are both provided to a trainable (or constructible) data model 7530. It will be understood that, in some examples, at least some of the various sensing modalities of the known inputs 7510 may be external to the patient. In some examples, the known output 7540 may comprise a determined sleep-wake status 7542 (e.g. such as used to determine initial sleep onset, WASO, and sleep onset after WASO), which may comprise any number of internally measurable and/or externally measurable physiologic parameters used for determining a sleep-wake status, such as but not limited to any one of (or combinations of) EEG, EOG, EMG, ECG, cardiac information, respiratory information, motion/activity, posture, etc.

As further shown in FIG. 41, in some examples at least some known inputs (obtained via the accelerometer or other sensors) may comprise a wide variety of sensed physiologic signals and/or information (e.g. sensing portion 2000) such as, but not limited to, cardiac information 7512, respiratory information 7514, motion/activity information 7516, posture information 7518, and/or other information 7519. It will be understood that these inputs are mere examples, and that the known inputs (from the accelerometer signal or other sensors) may comprise any sensed physiologic information pertinent to determining a sleep-wake status.

By providing such known inputs (7510) and known outputs (7540) to the trainable data model 7530, a trained data model 7631 (FIG. 42) may be obtained. In some examples, just one or some of the known inputs 7510 may be used, while all of the known inputs 7510 may be used in some examples. As noted elsewhere, the trainable/trained data model (7530, 7631) may comprise a deep learning model.

FIG. 42 is a diagram schematically representing an example method 7600 (and/or example device) for using a trained (or constructed) data model 7631 for determining sleep-wake status (e.g. initial sleep onset, WASO, and sleep onset after WASO) using internal measurements, such as (but not limited to) via an accelerometer (e.g. implantable and/or external) in some examples, and/or other internal or external measurements such as any one or more of the sensing modalities described within and throughout the present disclosure. As shown in FIG. 42, currently sensed inputs 7611 are fed into the trained data model 7631, which then produces a determinable output 7641, such as a current sleep-wake status determination 7643, which is based on the current inputs 7611. In some examples, the current inputs 7611 correspond to the same type and/or number of known inputs 7510 (FIG. 41) used to train the data model. In some examples, just one or some of the current inputs 7611 may be used, while all of the current inputs 7611 may be used in some examples.

As previously noted, once the trained data model 7631 is obtained, in some examples it is imported into and/or otherwise forms part of control portion 4000 in FIG. 34A (and/or care engine 2500 in FIG. 32) In some examples, other information 7519 (shown in FIGS. 41-42) may comprise input such as from external sensors associated with a remote control 4340, an app 4330 on mobile consumer device 4320, etc. (as shown in FIG. 36 and FIG. 34B) and/or associated with remote, app, physical parameters 3012, 3013, 3018 in FIG. 32. The external sensors/input may comprise ambient light, movement/operation of the remote control or of the app/mobile consumer device, etc. Other input may comprise time of day, time zone, geographic latitude, etc. as previously described in association with at least FIGS. 26E-26F, temporal parameter 3014 (FIG. 32), boundary parameter 3016 (FIG. 32), and the like regarding input used to at least partially determine sleep-wake status according to detecting a probability of sleep and/or a probability of wakefulness.

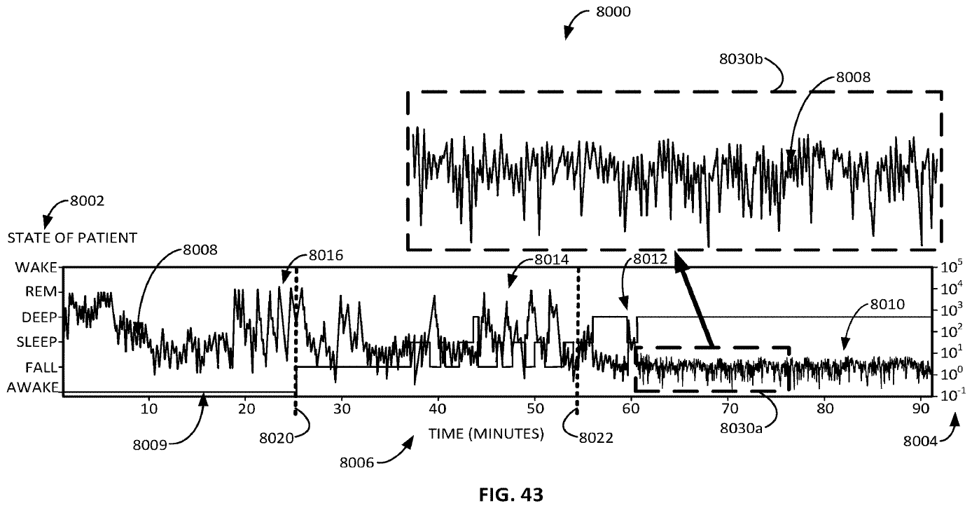

In some examples, implementing at least some aspects of the example methods and/or devices described in association with FIGS. 1A-42 may comprise use of, determining at least some of the information in, and/or implementing the methods in the examples of FIGS. 43-45B. Moreover, the examples of FIGS. 43-45B also may comprise an example implementation of at least some of the features of the example methods and/or devices associated with FIGS. 1A-42.

FIG. 43 is a chart 8000 schematically representing an example motion signal of a patient over 90 minutes. In some examples, the motion signal may be obtained from a sensing element (e.g. internal element 128 of FIG. 1B and/or external sensor 171, 150 of FIG. 1B) or a sensing portion (e.g. 2000 of FIG. 30A). In some examples, the motion signal may correspond to measured signal 284 of FIG. 3A or measured signal 294 of FIG. 3B. Chart 8000 includes a state of the patient on a first vertical axis 8002, including an awake (AWAKE) state, a falling asleep (FALL) state, a sleeping (SLEEP) state, a deep sleep (DEEP) state, a REM sleep (REM) state, and a waking (WAKE) state. Chart 8000 also includes milli-g's per second (mg/s) on a logarithmic scale on a second vertical axis 8004 and time in minutes on the horizontal axis 8006. Chart 8000 includes a motion signal 8008 over time corresponding to the logarithmic scale and a patient state signal 8009 over time corresponding to the state of the patient. The patient state signal 8009 may be derived from the motion signal 8008. Portion 8030a of the motion signal 8008 is magnified at 8030b.

In some examples, the motion signal 8008 may be obtained from a three axis accelerometer by low pass (anti-alias) filtering the X, Y, Z components and downsampling the filtered components. The filtered components may be downsampled to, for example, a 2 Hz sample rate. By downsampling the filtered components, the power consumption for processing the motion signal may be reduced. In some examples, instead of downsampling the accelerometer components, the accelerometer may directly provide X, Y, Z component samples at the 2 Hz sample rate. The downsampled X, Y, Z components are low pass filtered and differentiated using a single filter (similar to a bandpass filter) to generate velocity components. In some examples, the low pass section of the filter may have a cut-off of about 0.06 Hz, such that the average velocity over a period of about 15 seconds is calculated. The root sum square (RSS) of the X, Y, Z velocity components may then be calculated to generate the motion signal 8008.

The magnitude of the peaks in the motion signal 8008 may relate to different types of patient motion. Peak values equal to about 10 mg/s as indicated at 8010 are indicative of cardiac and respiratory motion. Peak values equal to about 100 mg/s as indicated at 8012 are indicative of respiratory events. Peak value equal to about 103 mg/s as indicated at 8014 are indicative of arousals. Peak values equal to about 104 mg/s as indicated at 8016 are indicative of posture changes or awake motions.

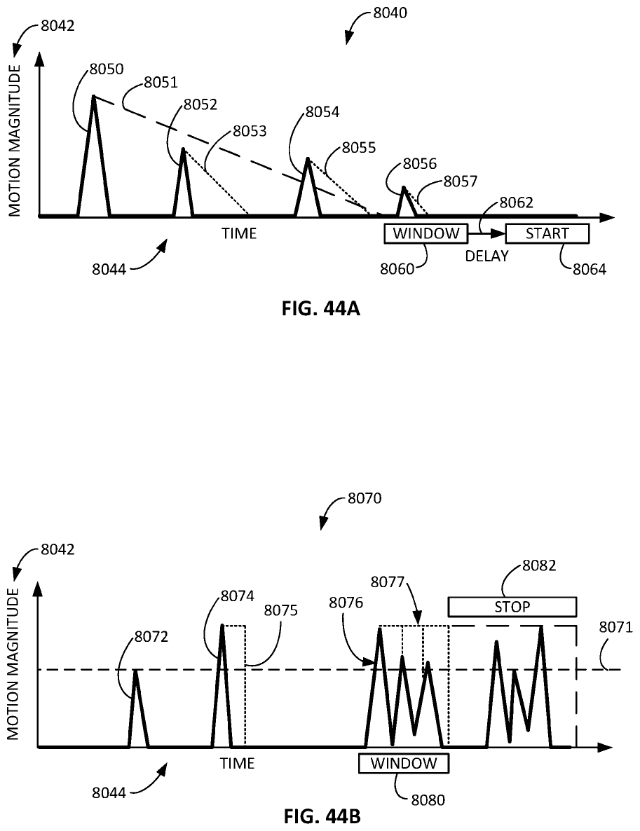

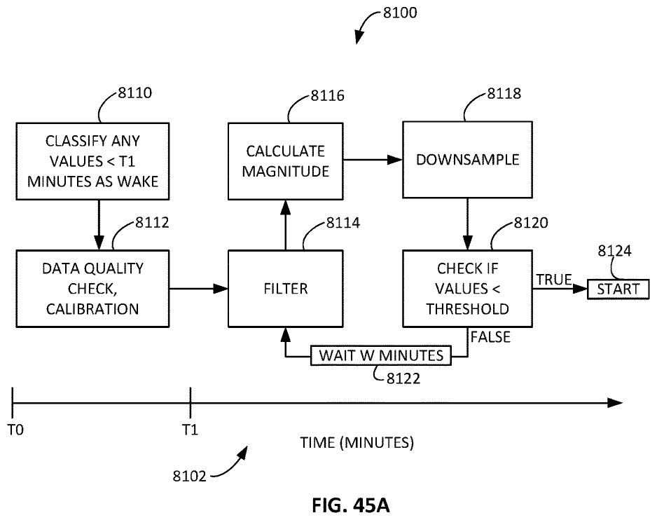

Accordingly, as described in more detail below with reference to FIGS. 44A and 44B, the magnitude and persistence of the peaks of the motion signal 8008 are indicative of the state of the patient. Prior to time 8020, as indicated by state signal 8009, the patient is determined to be awake. Between times 8020 and 8022, the patient is determined to be falling asleep, and after time 8022, the patient is determined to be asleep.

FIG. 44A is a chart 8040 schematically representing an example motion signal (e.g. 8008 of FIG. 43) for detecting sleep onset of a patient. In some examples, chart 8040 illustrates an example implementation for automatically initiating electrical stimulation in response to detecting initial sleep onset or sleep onset after WASO as previously described with reference to method 310 of FIG. 4B. Chart 8040 includes motion magnitude on the vertical axis 8042 versus time on the horizontal axis 8044. A timer reset method may be used to detect sleep onset by monitoring a reduction in the magnitude of the motion signal peaks over time. Each peak in the motion signal casts a shadow implemented by a timer that is counting down. Larger peaks as indicated at 8050 cast longer shadows as indicated at 8051. Smaller peaks as indicated at 8052, 8054, and 8056 cast shorter shadows as indicated at 8053, 8055, and 8057, respectively. The fraction of shadowed time over the last N minutes indicated by window 8060 is monitored, where “N” may be within a range between 3 and 10. When the window 8060 is completely covered by a shadow from a large peak, this fraction is 100%. When the window is partially covered by a one or more shadows from smaller peaks, this fraction drops to a lower value. When the shadowed fraction drops below a threshold (e.g., 5%), a delay period is initiated as indicated at 8062. If no large peaks (e.g., peaks casting a shadow covering more than a predefined percentage (e.g. 5%, 10%, 15%) of the current window) are detected during the delay period 8062, the patient is determined to be asleep (e.g. sleep onset is detected) and therapy may be started as indicated at 8064. If a large peak is detected during the delay period 8062, the process is restarted with the current window.

FIG. 44B is a chart 8070 schematically representing an example motion signal (e.g. 8008 of FIG. 43) for detecting wake after sleep onset (WASO) of a patient. In some examples, chart 8070 illustrates an example implementation for automatically pausing and/or stopping electrical stimulation in response to detecting WASO as previously described with reference to method 312 of FIG. 4B. Chart 8070 includes motion magnitude on the vertical axis 8042 versus time on the horizontal axis 8044. Therapy may be automatically paused or automatically stopped based on the motion signal. WASO may be detected in response to a persistent occurrence of large peaks of the motion signal over a window of time. Persistent motion may distinguish waking from minor arousals. At the same time, therapy should be paused quickly in response to a patient waking so that the patient does not need to manually pause the therapy. Therefore, therapy may be automatically paused for a brief time in response to a single motion, but quickly restarted if persistent motion is not detected.

Peaks below a threshold 8071 as indicated by peak 8072 (e.g. due to a respiration event) result in determining the patient remains asleep and therapy may continue. Peaks above the threshold 8071 as indicated by peak 8074 result in therapy being paused for a window of time (e.g. 30 seconds) as indicated at 8075. If no peaks exceed the threshold during this window of time, therapy is restarted. If another peak exceeds the threshold during this window of time as indicated by peaks 8076, the pause continues for another window of time as indicated by windows 8077. If therapy is paused for a threshold number of windows (e.g. three in this example) as indicated by window 8080, the patient is determined to be awake (e.g. WASO is detected) and therapy is stopped as indicated at 8082. With therapy stopped, the timer reset method described above with reference to FIG. 44A may then be used to detect sleep onset after WASO. In some examples, the threshold 8071 may be selected by a physician and/or based on a data model (e.g. 7631 of FIG. 42).

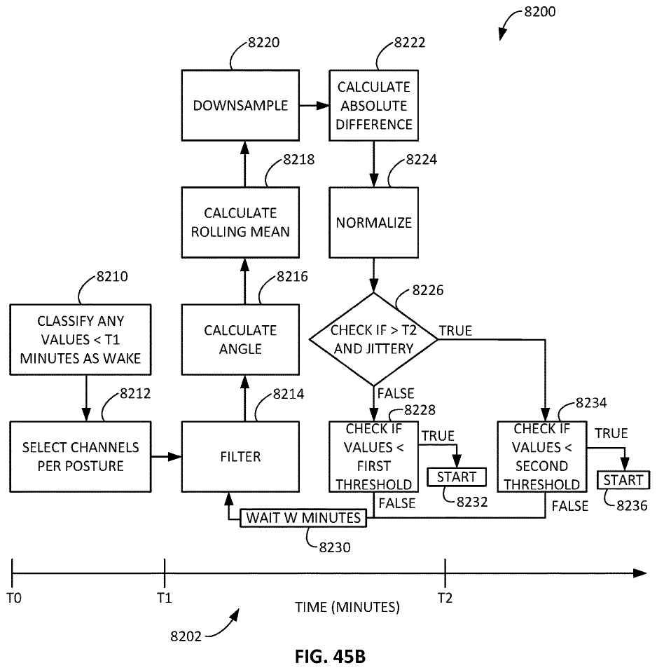

FIG. 45A is a diagram schematically representing an example method 8100 for determining sleep onset of a patient. In some examples, method 8100 is an example implementation of the timer reset method previously described with reference to FIG. 44A. In some examples, method 8100 may be implemented by a control portion, such as control portion 4000 of FIG. 34A. Time in minutes is indicated at 8102. The input to method 8100 may be a sensor (e.g. a three axis accelerometer) signal indicative of motion of a patient, such as from an internal sensing element 128 of FIG. 1B, an external sensor 171, 150 of FIG. 1B, and/or a sensing portion 2000 of

FIG. 30A. At time T0, it is determined that the patient intends to sleep, such as by the patient manually indicating they intend to sleep (e.g. via a remote control, mobile device, user interface, etc.) or by sensing the patient intends to sleep via an internal sensor(s) (e.g., accelerometer, gyroscope, microphone, etc.) and/or an external sensor(s) (e.g. accelerometer, light sensor, motion sensor, sleep mat, wearable device, pneumatic sensor, low power radar sensor, etc.). In response to the determination that the patient intends to sleep at time T0, at 8110 any sensor signal values received prior to a time T1 are classified as indicative of an awake state. Time T1 may be selected such that T1 minus T0 equals a minimum amount of time for the patient to fall asleep, such as within a range between 10 minutes and 30 minutes. Accordingly, for a first predetermined period (i.e. T1-T0) from determining the patient intends to sleep, the patient is determined to be awake. At 8112, this first predetermined period may include a data quality check and a calibration period. During this calibration period, any values of the sensor signal are classified as being indicative of an awake state. Sensor signal values collected during this calibration period may be checked against known values of a data model (e.g. 7631 of FIG. 42) to ensure that the sensor signal quality is sufficient to detect sleep. If the sensor signal quality is sufficient, the calibration period may be used to further train a data model (e.g. 7530 of FIG. 41). After the predetermined period at time T1, active sleep detection may begin.

Steps 8114-8124 process the most recent N minutes of the sensor signal, where “N” is within a range between 0.5 and 10 (e.g. 7). At 8114, the sensor signal is filtered (e.g. low pass filtered or band pass filtered). For example, X, Y, Z components of a 10-50 Hz three channel accelerometer sensor signal may be bandpass filtered to provide a filtered signal. At 8116, motion magnitudes are calculated from the filtered signal to provide a motion magnitude signal. In some examples, the motion magnitudes may be calculated over a previous M minutes, where “M” is within a range between 3 and 10 (e.g. 7). For example, the root sum square (RSS) of the X, Y, Z, components of the filtered three channel accelerometer sensor signal may be calculated using all three channels to provide the motion magnitude signal. At 8118, the motion magnitude signal over the previous M minutes is downsampled to provide a measurement every D seconds over the previous M minutes, where “D” is within a range between 1 and 10, such as 5 (e.g. 1/5 Hz). At 8120, the values of the measurements from 8118 are checked to determine if all the values are below a threshold (e.g. 6). In response to any of the values being above the threshold, the patient is determined to be awake and at 8122 the process waits W minutes, where “W” is within a range between 0.5 and 2 minutes, before repeating the process beginning at 8114. In response to all the values being below the threshold, the patient is determined to be asleep (e.g. sleep onset is detected) and therapy may be started at 8124.

FIG. 45B is a diagram schematically representing an example method 8200 for determining sleep onset of a patient. In some examples, method 8200 is an example implementation for automatically initiating electrical stimulation in response to detecting initial sleep onset or sleep onset after WASO as previously described with reference to method 310 of FIG. 4B. In some examples, method 8200 may be implemented by a control portion, such as control portion 4000 of FIG. 34A. Time in minutes is indicated at 8202. The input to method 8200 may be a sensor (e.g. a three axis accelerometer) signal indicative of motion of a patient, such as from an internal sensing element 128 of FIG. 1B, an external sensor 171, 150 of FIG. 1B, and/or a sensing portion 2000 of FIG. 30A. At time T0, it is determined that the patient intends to sleep, such as by the patient manually indicating they intend to sleep (e.g. via a remote control, mobile device, user interface, etc.) or by sensing the patient intends to sleep via an internal sensor(s) (e.g., accelerometer, gyroscope, microphone, etc.) and/or an external sensor(s) (e.g. accelerometer, light sensor, motion sensor, sleep mat, wearable device, pneumatic sensor, low power radar sensor, etc.). In response to the determination that the patient intends to sleep at T0, at 8210 any sensor signal values received prior to a time T1 are classified as indicative of an awake state. Time T1 may be selected such that T1 minus T0 equals a minimum amount of time for the patient to fall asleep, such as within a range between 10 minutes and 30 minutes. Accordingly, for a first predetermined period (i.e. T1-T0) from determining the patient intends to sleep, the patient is determined to be awake. At 8212, this first predetermined period may be used to select a subset of sensor signal channels based on the posture of the patient. For example, if the patient is prone during the first two minutes and then on their left side during the remaining time, the accelerometer channels associated with those postures may be selected (e.g. the channel(s) having the largest values for each posture). In some examples, this first predetermined period may also include a data quality check and calibration period as described above with reference to 8112 of FIG. 45A. After the predetermined period at time T1, active sleep detection may begin.

Steps 8214-8236 process the most recent N minutes of the sensor signal, where “N” is within a range between 0.5 and 10 (e.g. 7). At 8214, the sensor signal is filtered (e.g. low pass filtered). For example, X, Y, Z components of a 10-50 Hz three channel accelerometer sensor signal may be low pass filtered to provide a filtered signal. At 8216, angles (e.g. relative to gravity) are calculated from the filtered signal corresponding to each selected channel for the time period in which the patient is in that posture to generate an angle signal. At 8218, a rolling mean of the angle signal from 8216 is calculated. In some examples, a R second rolling mean of the angles is calculated over the previous M minutes, where “R” is within a range between 1 and 10 (e.g. 5) and “M” is within a range between 3 and 10 (e.g. 7). At 8220, the rolling mean signal is downsampled to provide a measurement every D seconds over the previous M minutes, where “D” is within a range between 1 and 10, such as 5 (e.g. 1/5 Hz). At 8222, the absolute difference between successive measurements of the downsampled signal are calculated. At 8224, the absolute differences between successive measurements are normalized according to a mean and standard deviation of a data model (e.g. 7631 of FIG. 42).

At 8226, the current time is compared to a time T2. Time T2 may be selected such that T2 minus T0 equals a maximum amount of time for the patient to fall asleep, such as within a range between 30 minutes and 60 minutes. In response to the current time being less than T2, at 8228 the normalized values from 8224 are checked to determine if all the values are below a first threshold (e.g. 0). In response to any of the values being above the first threshold, the patient is determined to be awake and at 8230 the process waits W minutes, where “W” is within a range between 0.5 and 2 minutes, before repeating the process beginning at 8214. In response to all the values being below the first threshold, the patient is determined to be asleep (e.g. sleep onset is detected) and therapy may be started at 8232. In response to the current time being greater than T2 (e.g. greater than a second predetermined period) and the patient being jittery (e.g. substantial variations in the normalized values from 8224) at 8226, at 8234 the normalized values from 8224 are checked to determine if all the values are below a second threshold (e.g. 1). In response to any of the values being above the second threshold, the patient is determined to be awake and at 8230 the process waits W minutes before repeating the process beginning at 8214. In response to all the values being below the second threshold, the patient is determined to be asleep (e.g. sleep onset is detected) and therapy may be started at 8236. Method 8200 accounts for cases where the patient is experiencing jittery or restless sleep by increasing the threshold to detect sleep onset after the second predetermined period of time (e.g. T2-T0).

Although specific examples have been illustrated and described herein, a variety of alternate and/or equivalent implementations may be substituted for the specific examples shown and described without departing from the scope of the present disclosure. This application is intended to cover any adaptations or variations of the specific examples discussed herein.

1-33. (canceled)

34. A medical device comprising:
at least one sensor to sense physiologic information; and
a control portion configured to:
receive a plurality of inputs each corresponding to a different sleep-wake determination parameter, the plurality of inputs comprising the sensed physiologic information;
detect initial sleep onset based on at least a first subset of the plurality of inputs and first threshold values for the at least first subset of the plurality of inputs;
detect wake after sleep onset (WASO) based on at least a second subset of the plurality of inputs and second threshold values for the at least second subset of the plurality of inputs; and
detect sleep onset after WASO based on at least a third subset of the plurality of inputs and third threshold values for the at least third subset of the plurality of inputs,
wherein the third threshold values are different from the first threshold values and the second threshold values.

35. The medical device of claim 34, wherein the medical device comprises an implantable medical device.

36. The medical device of claim 34, further comprising:
a pulse generator to apply electrical stimulation,
wherein the control portion is configured to:
automatically initiate a first function of the pulse generator in response to detecting initial sleep onset or sleep onset after WASO; and
automatically pause the first function of the pulse generator in response to detecting WASO.

37. The medical device of claim 36, wherein the first function of the pulse generator is to apply electrical stimulation to an upper airway patency-related nerve.

38. The medical device of claim 34, wherein the first threshold values and/or the third threshold values are time dependent.

39. The medical device of claim 38, wherein, during a sleep period, the third threshold values are initially at minimum values at a start of the sleep period, increase from the minimum values to maximum values, and then decrease from the maximum values back to the minimum values by the end of the sleep period.

40. The medical device of claim 34, wherein the second subset of the plurality of inputs is different from the first subset of the plurality of inputs.

41. The medical device of claim 40, wherein the first subset of the plurality of inputs comprises at least one of heart rate variability or body temperature, and the second subset of the plurality of inputs comprises at least one of activity or locomotor inactivity during sleep (LIDS).

42. The medical device of claim 34, wherein the third subset of the plurality of inputs comprises at least one of activity, locomotor inactivity during sleep (LIDS), a light sensor signal, or time elapsed since initial sleep onset.

43. The medical device of claim 34, wherein the first subset of the plurality of inputs is different from the third subset of the plurality of inputs.

44. The medical device of claim 34, wherein the at least one sensor to sense physiologic information comprises at least one of:
an accelerometer sensor; or a temperature sensor.

45. The medical device of claim 34, wherein the at least one sensor to sense physiologic information is implantable.

46. The medical device of claim 34, further comprising:
at least one sensor external to a patient's body to sense environmental information;
wherein the at least one sensor to sense environmental information comprises at least one of:
a light sensor;
a noise sensor; or
a movement sensor.

47. A medical device comprising:
at least one sensor to generate a sensor signal indicative of motion of a patient; and
a control portion configured to detect initial sleep onset by:
calculating magnitudes of the sensor signal to generate a motion magnitude signal;
in response to each respective peak of the motion magnitude signal, starting a respective countdown timer having an initial value corresponding to a magnitude of the respective peak, the respective countdown timer representing a respective shadow cast by the respective peak;
determining a fraction of a moving window covered by the shadows;
starting a delay period in response to the fraction dropping below a first threshold value; and
in response to the fraction remaining below the first threshold value for the delay period, detecting initial sleep onset.

48. The medical device of claim 47, wherein the control portion is configured to detect wake after sleep onset (WASO) based on the motion magnitude signal.

49. The medical device of claim 48, wherein the control portion is configured to detect WASO by:
starting a window of time in response to a respective peak of the motion magnitude signal exceeding a second threshold value; and
in response to a selected number of respective peaks of the motion magnitude signal exceeding the second threshold value prior to the window of time expiring, detecting WASO.

50. The medical device of claim 47, wherein the at least one sensor comprises an accelerometer.

51. A medical device comprising:
at least one sensor to generate a sensor signal indicative of motion of a patient; and
a control portion configured to detect wake after sleep onset (WASO) by:
calculating magnitudes of the sensor signal to generate a motion magnitude signal;
starting a window of time in response to a respective peak of the motion magnitude signal exceeding a second threshold value; and
in response to a selected number of respective peaks of the motion magnitude signal exceeding the second threshold value prior to the window of time expiring, detecting WASO.

52. The medical device of claim 51, wherein the control portion is configured to detect initial sleep onset based on the motion magnitude signal; and
detect sleep onset after WASO based on the motion magnitude signal.

53. The medical device of claim 52, wherein the control portion is configured to detect initial sleep onset by:
in response to each respective peak of the motion magnitude signal, starting a respective countdown timer having an initial value corresponding to a magnitude of the respective peak, the respective countdown timer representing a respective shadow cast by the respective peak;
determining a fraction of a moving window covered by the shadows;
starting a delay period in response to the fraction dropping below a first threshold value; and
in response to the fraction remaining below the first threshold value for the delay period, detecting initial sleep onset.
